(19) United States
(12) Patent Application Publication
(10) Pub. No.: US 2024/0335505 A1
(43) Pub. Date: Oct. 10, 2024

(54) PRODUCTS AND METHODS FOR PROMOTING MYOGENESIS

(71) Applicant: Ecole Polytechnique Federale de Lausanne (EPFL), Lausanne (CH)

(72) Inventors: Martin Wohlwend, Renens (CH); Johan Auwerx, Buchillon (CH); Ulrik Wisloff, Jonsvatnet (NO); Jose Bianco Moreira, Seritinga-MG (BR)

(21) Appl. No.: 18/271,503

(22) PCT Filed: Feb. 10, 2022
(86) PCT No.: PCT/EP22/53229

§ 371 Date: Jul. 10, 2023

(30) Foreign Application Priority Data
Feb. 11, 2021 (GB) 2101932.8

(51) Int. Cl. A61K 38/17; A01K 67/0275; A61K 31/409; A61K 48/00; A61P 21/00; C12N 5/077; C12N 15/113; C12N 15/115; C12Q 1/6876

(52) U.S. Cl. CPC A61K 38/1709; A01K 67/0275; A61K 31/409; A61K 48/0033; A61P 21/00; C12N 5/0658; C12N 15/113; C12N 15/115; C12Q 1/6876; C12N 2310/11; C12N 2310/12; C12N 2310/14; C12N 2310/531; C12N 2506/1323; C12N 2510/00; C12Q 2600/124; C12Q 2600/156

(57) ABSTRACT

The present invention relates generally to the field of long non-coding RNAs and transcription factors and modulation of their expression for use in medicine and agriculture, such as the treatment and prevention of diseases associated with muscle atrophy and the production of livestock. More particularly, the invention provides agonists of Cytor, a long non-coding RNA, e.g. agents that increase the amount of Cytor RNA in skeletal muscle as well as antagonists for Teadl, a transcription factor, e.g. agents that decrease the amount of Teadl RNA or protein in skeletal muscle and their use in therapy.

### RELATED APPLICATIONS

This application is the § 371 National Stage of International Patent Application No.: PCT/EP2022/053229, filed Feb. 10, 2022, which claims priority from United Kingdom Application No.: 2101932.8, filed on Feb. 11, 2021. The contents each of the related applications are hereby incorporated by reference in their entirety.

### SEQUENCE LISTING

This application contains a Sequence Listing in ASCII computer readable form. The Sequence Listing is incorporated herein by reference. Said ASCII file, created on Mar. 12, 2024, is named VPH-02401_SL.txt and is 1,008,819 bytes in size.

### FIELD OF THE INVENTION

The present invention relates generally to the field of long non-coding RNAs and transcription factors and modulation of their expression for use in medicine and agriculture, such as the treatment and prevention of diseases associated with muscle atrophy and the production of livestock. More particularly, the invention provides agonists of Cytor, a long non-coding RNA, e.g. agents that increase the amount of Cytor RNA in skeletal muscle as well as antagonists for Tead1, a transcription factor, e.g. agents that decrease the amount of Tead1 RNA or protein in skeletal muscle and their use in therapy. Such agonists and/or antagonists have therapeutic uses in diseases and conditions associated with muscle atrophy and dysfunction. The invention also contemplates the modulation of Cytor RNA and/or Tead1 RNA or protein in myoblasts and skeletal muscle, e.g. via genetic modification or gene therapy, in therapy and to enhance meat production, both in vitro and in vivo. The invention also provides a modified non-human animal with increased levels of Cytor RNA and/or decreased levels of or no Tead1 RNA or protein, products derived therefrom and methods of producing such animals. Various diagnostic methods relating to Cytor expression and/or Tead1 expression are also provided.

### BACKGROUND TO THE INVENTION

The non-coding genome encompasses a variety of transcribed RNA molecules, e.g. miRNAs, siRNAs, piRNAs etc. One class of non-coding RNA molecules is termed long non-coding RNAs (lncRNAs) and refers to polyadenylated RNA molecules that are >200 nucleotides in length. LncRNAs represent a large fraction of transcribed but not translated genes and have been associated with regulation of multiple cellular processes, including cellular plasticity. Transcription of lncRNAs it thought to be tightly regulated, both spatially and temporally, and changes in the expression of some lncRNAs have been linked with various diseases, including cancer. However, the endogenous functions of most lncRNAs remain unclear.

Transcription factors (TFs) are proteins that controls the rate of transcription of genetic information from DNA to messenger RNA or non-coding RNA, by binding to a specific DNA sequence. The function of TFs is to regulate the expression genes in the right cell at the right time and in the right amount throughout the life of the cell and the organism. Groups of TFs function in a coordinated fashion to direct cell division, cell growth, and cell death throughout life; cell migration and organization (body plan) during embryonic development; and intermittently in response to signals from outside the cell, such as a hormone. There are up to 1600 TFs in the human genome.

Skeletal muscle is one of the organs most affected by ageing exemplified by estimated yearly losses of ˜1% muscle mass and 3% muscle strength in the elderly, resulting in an accumulated net loss of >30% muscle mass during ageing. Sarcopenia, a degenerative geriatric syndrome recently recognized as a disease by the WHO, is defined as the progressive deterioration of skeletal muscle mass and function with age. With a current estimated prevalence of 10% in the general population—a number that will increase with the ageing population—sarcopenia is already one of the leading causes of frailty, loss of independence and quality of life in aged individuals and constitutes a risk factor for all-cause mortality.

Skeletal muscle ageing has repeatedly been shown to primarily affect fast-twitch, type II muscle fibres. Aerobic type IIa and glycolytic type IIb muscle fibres decrease with age in number and fibre area compared to slow-twitch, type I muscle fibres in humans and rodents, thereby primarily contributing to the age-inflicted loss in muscle mass and strength. Despite knowledge of the muscle fibres primarily affected by aging, in the absence of a more comprehensive knowledge of the molecular players involved in type II myogenesis, there has been limited progress in the development of pharmaceutical agents needed to treat the decline in muscle function and mass observed in ageing and other diseases associated with muscle atrophy.

Exercise is a well-known modulator of muscle mass and function, in young and aged subjects, and has been documented to both prevent and ameliorate sarcopenia. Interestingly, exercise-mediated maintenance of muscle mass and strength at old age is largely attributed to promotion of type II muscle fibres. However, mechanisms underlying the exercise-mediated biogenesis of type II fibres in the ageing muscle are not well elucidated. Moreover, as exercise-based physiotherapy is not suitable for all subjects with sarcopenia or other diseases associated with muscle atrophy, there is a need for alternative (e.g. pharmacological) therapies that target type II muscle fibres, e.g. for improved growth and function.

### SUMMARY OF THE INVENTION

The present inventors have determined that Cytor, a conserved lncRNA, is an exercise-induced lncRNA in mouse, rat, and human skeletal muscle. The inventors have shown through genetic manipulation of Cytor expression in vitro that Cytor regulates myoblast differentiation into type II myotubes. Moreover, as described in the Examples below, CRISPR-dCas9 mediated rescue of endogenous Cytor expression in aged mice ameliorates ageing-inflicted impairments in skeletal muscle function, mass and proportion of type II fibres. Furthermore, direct Cytor overexpression using CRISPR-dCas9 in aged human myoblasts improves differentiation into type II myotubes.

Using C. elegans as a model of ageing, the inventors observed that forced expression of human Cytor under a muscle-specific promoter rejuvenates muscle morphology and improves movement in aged worms, demonstrating the functional potency of human Cytor. The inventors have also identified a causal cis-eQTL, rs74360724, located within an enhancer regulatory region, whose minor allele A promotes Cytor expression in human skeletal muscle and associates with fitness in aged individuals. Similarly, the identified a causal cis-eQTL, rs79200838, located within the Tead1 gene, whose minor allele T reduces Tead1 expression in human skeletal muscle and associates with fitness in aged individuals.

Whilst not wishing to be bound by theory, it is hypothesised that Cytor reduces chromatin accessibility at binding sites of the transcription factor Tead1. Tead1 silencing has previously been shown to specifically promote type II muscle fibres in vitro and in vivo. Accordingly, it is thought that Cytor's effect on differentiation of myoblasts into type II myotubes is dependent on Tead1.

It is furthermore demonstrated in the appended examples that the co-overexpressed of Cytor and Tead1 reduces myotube area, myofusion, and abolishes expression of type II myosin isoforms. On the other hand, the silencing of Tead1 mRNA expression by a specific small interfering RNA (siRNA) or the inhibition of Tead1 protein by the Tead1 inhibitor verteporfin was sufficient to increase myotube area, myofusion, and induce the expression of type II myosin isoforms.

Based on these experimental findings, the inventors propose that modulating Cytor activity and/or Tead1 activity provides an attractive therapeutic strategy to boost muscle growth and function in diseases and conditions featuring muscle atrophy, such as sarcopenia. Gene therapy and/or pharmacological agents that promote Cytor expression and/or function and/or decrease or prevent Tead1 expression and/or function are expected to improve myogenesis and hence, skeletal muscle growth and function.

Accordingly, in one aspect, the present invention provides a Cytor agonist (e.g. an agent that increases the amount of Cytor RNA in skeletal muscle) and/or Tead1 antagonist (e.g. an agent that decreases the amount of or entirely depletes Tead1 RNA or protein in skeletal muscle) for use in therapy.

In this connection the conjunction “and/or” means that the Cytor agonist and the Tead1 antagonist may be used alone or in combination. In this respect it is particularly preferred that the Cytor agonist is used and optionally in addition the Tead1 antagonist. It is expected that the Tead1 antagonist enhances the desired therapeutic effect of the Cytor agonist, and vice versa.

Alternatively viewed, the present invention provides a method for treating and/or preventing a disease or condition (e.g. disease or condition associated with muscle atrophy) in a subject, which comprises the step of administering a Cytor agonist (e.g. an agent that increases the amount of Cytor RNA in skeletal muscle) and/or Tead1 antagonist (e.g. an agent that decreases the amount of or entirely depletes Tead1 RNA or protein in skeletal muscle) according to the invention to the subject.

The invention also provides the use of a Cytor agonist (e.g. an agent that increases the amount of Cytor RNA in skeletal muscle) and/or Tead1 antagonist (e.g. an agent that decreases the amount of or entirely depletes Tead1 RNA or protein in skeletal muscle) in the manufacture of a medicament for treating and/or preventing a disease or condition (e.g. disease or condition associated with muscle atrophy) in a subject.

In another aspect, the present invention provides a Cytor agonist (e.g. an agent that increases the amount of Cytor RNA in skeletal muscle) and/or Tead1 antagonist (e.g. an agent that decreases the amount of or entirely depletes Tead1 RNA or protein in skeletal muscle) for use in promoting myogenesis in skeletal muscle of a subject.

Alternatively viewed, the present invention provides a method for promoting myogenesis in skeletal muscle of a subject, which comprises the step of administering a Cytor agonist (e.g. an agent that increases the amount of Cytor RNA in skeletal muscle) and/or Tead1 antagonist (e.g. an agent that decreases the amount of or entirely depletes Tead1 RNA or protein in skeletal muscle) according to the invention to the subject.

The invention also provides the use of a Cytor agonist (e.g. an agent that increases the amount of Cytor RNA in skeletal muscle) and/or Tead1 antagonist (e.g. an agent that decreases the amount of or entirely depletes Tead1 RNA or protein in skeletal muscle) in the manufacture of a medicament for promoting myogenesis in skeletal muscle of a subject.

In another aspect, the present invention provides a Cytor agonist (e.g. an agent that increases the amount of Cytor RNA in skeletal muscle) and/or Tead1 antagonist (e.g. an agent that decreases the amount of or entirely depletes Tead1 RNA or protein in skeletal muscle) for use in increasing skeletal muscle mass in a subject.

Alternatively viewed, the present invention provides a method for increasing skeletal muscle mass in a subject, which comprises the step of administering a Cytor agonist (e.g. an agent that increases the amount of Cytor RNA in skeletal muscle) and/or Tead1 antagonist (e.g. an agent that decreases the amount of or entirely depletes Tead1 RNA or protein in skeletal muscle) according to the invention to the subject.

The invention also provides the use of a Cytor agonist (e.g. an agent that increases the amount of Cytor RNA in skeletal muscle) and/or Tead1 antagonist (e.g. an agent that decreases the amount of or entirely depletes Tead1 RNA or protein in skeletal muscle) in the manufacture of a medicament for increasing skeletal muscle mass in a subject.

In another aspect, the present invention provides a Cytor agonist (e.g. an agent that increases the amount of Cytor RNA in skeletal muscle) and/or Tead1 antagonist (e.g. an agent that decreases the amount of or entirely depletes Tead1 RNA or protein in skeletal muscle) for use in preventing or treating skeletal muscle atrophy and/or skeletal muscle dysfunction in a subject.

Alternatively viewed, the present invention provides a method for preventing or treating skeletal muscle atrophy and/or skeletal muscle dysfunction in a subject, which comprises the step of administering a Cytor agonist (e.g. an agent that increases the amount of Cytor RNA in skeletal muscle) and/or Tead1 antagonist (e.g. an agent that decreases the amount of or entirely depletes Tead1 RNA or protein in skeletal muscle) according to the invention to the subject.

The invention also provides the use of a Cytor agonist (e.g. an agent that increases the amount of Cytor RNA in skeletal muscle) and/or Tead1 antagonist (e.g. an agent that decreases the amount of or entirely depletes Tead1 RNA or protein in skeletal muscle) in the manufacture of a medicament for preventing or treating skeletal muscle atrophy and/or skeletal muscle dysfunction in a subject.

In another aspect, the present invention provides a Cytor agonist (e.g. an agent that increases the amount of Cytor RNA in skeletal muscle) and/or Tead1 antagonist (e.g. an agent that decreases the amount of or entirely depletes Tead1 RNA or protein in skeletal muscle) for use in improving skeletal muscle function in a subject.

Alternatively viewed, the present invention provides a method for improving skeletal muscle function in a subject, which comprises the step of administering a Cytor agonist (e.g. an agent that increases the amount of Cytor RNA in skeletal muscle) and/or Tead1 antagonist (e.g. an agent that decreases the amount of or entirely depletes Tead1 RNA or protein in skeletal muscle) according to the invention to the subject.

The invention also provides the use of a Cytor agonist (e.g. an agent that increases the amount of Cytor RNA in skeletal muscle) and/or Tead1 antagonist (e.g. an agent that decreases the amount of or entirely depletes Tead1 RNA or protein in skeletal muscle) in the manufacture of a medicament for improving skeletal muscle function in a subject.

The Cytor agonist and/or the Tead1 antagonist may find utility in improving or promoting various attributes of skeletal muscle simultaneously, e.g. an increase in muscle mass may also result in an increase/improvement of muscle function. Thus, in some embodiments the Cytor agonist and/or the Tead1 antagonist may result in a combination of any of the effects set out above. For instance, the Cytor agonist and/or Tead1 antagonist may be for use in promoting myogenesis and muscle function. Similarly, the Cytor agonist and/or the Tead1 antagonist may be for use in increasing skeletal muscle mass and function.

In connection with all the above medical uses and method of treatments the Tead1 antagonist preferably and in particular results in fast-twitch muscle fiber promotion. The two types of skeletal muscle fibers are slow-twitch (type I) and fast-twitch (type II). Slow-twitch muscle fibers support long distance endurance activities like marathon running, while fast-twitch muscle fibers support quick, powerful movements such as sprinting or weightlifting.

In a further aspect, the invention provides a modified (e.g. a genetically-modified) non-human animal which has an increased amount of Cytor RNA and/or decreased amount or no Tead1 RNA or protein in its skeletal muscle in comparison to a corresponding unmodified non-human animal.

In a still further aspect, the invention provides meat or a meat-containing product derived from the modified non-human animal of the invention.

In another aspect, the invention provides a method for producing (or generating) the modified non-human animal of the invention, the method comprising: (i) providing a non-human cell (e.g. a non-human pluripotent stem cell or germ cell) which has been genetically-modified such that myoblast cells derived from said cell express an increased amount of Cytor RNA or a decreased amount of or no Tead1 RNA or protein compared to unmodified myoblast cells; and
(ii) generating a genetically-modified non-human animal from said genetically-modified non-human cell (e.g. non-human pluripotent stem cell or germ cell).

In a further aspect, the invention provides an in vitro method for producing muscle fibres comprising culturing modified myoblasts which have increased amounts of Cytor RNA and/or a decreased amount of or no Tead1 RNA or protein in comparison to unmodified myoblasts under conditions suitable to produce muscle fibres.

Muscle fibres and/or meat produced by the in vitro method of the invention form a further aspect of the invention.

In another aspect, the invention provides a method for determining the risk of developing skeletal muscle atrophy (e.g. sarcopenia) in a subject comprising determining the genotype of the subject at the cis-eQTL rs74360724, wherein when the subject is heterozygous or homozygous for the G allele they have an increased risk of developing muscle atrophy (e.g. sarcopenia) compared to a subject that is homozygous for the A allele, and/or at the cis-eQTL rs79200838, wherein when the subject is heterozygous or homozygous for the T allele they have an decreased risk of developing muscle atrophy (e.g. sarcopenia) compared to a subject that is homozygous for the C allele.

In a further aspect, the invention provides a method for predicting the performance of a subject in an activity associated with fast-twitch muscle (e.g. an athlete, e.g. a sprinter) comprising determining the genotype of the subject at the cis-eQTL rs74360724, wherein when the subject is heterozygous or homozygous for the A allele they have an increased likelihood of outperforming a subject that is homozygous for the G allele, and/or at the cis-eQTL rs79200838, wherein when the subject is heterozygous or homozygous for the C allele they have an decreased of outperforming a subject that is homozygous for the T allele.

In another aspect, the invention provides a method for predicting the capability of a subject to produce fast-twitch muscle comprising determining the genotype of the subject at the cis-eQTL rs74360724, wherein when the subject is heterozygous or homozygous for the A allele they have an increased capability of producing fast-twitch muscle compared a subject that is homozygous for the G allele, and/or at the cis-eQTL rs79200838, wherein when the subject is heterozygous or homozygous for the C allele they have an decreased capability of producing fast-twitch muscle compared a subject that is homozygous for the T allele.

### DETAILED DESCRIPTION

The terms “Cytor” and “Cytor RNA” are used interchangeably herein and refer to a long non-coding RNA comprising a nucleotide sequence as set forth in any one of SEQ ID NOs: 1-15 or an orthologue or naturally-occurring variant thereof. Human Cytor is also known as “Cytoskeleton Regulator RNA”, “Long Intergenic Non-Protein Coding RNA 152”, “LINC00152”, “Chromosome 2 Open Reading Frame 59” and “Non-Protein Coding RNA 152”. Mouse Cytor is also known as “myeloid RNA regulator of BCL2L11 induced cell death” or “Morrbid”.

Notably, naturally-occurring splice variants of Cytor have been observed in human and mouse. SEQ ID NOs: 1-6 represent known common variants of human Cytor. Notably, the Ensembl database indicates that there may be at least 38 different isoforms of human Cytor. The most abundant human Cytor variant is thought to have the sequence set forth in SEQ ID NO: 1.

SEQ ID NOs: 7-12 represent known variants of mouse Cytor. Again, the Ensembl database indicates that there may be at least 50 different isoforms of mouse Cytor. The most abundant mouse Cytor variants are thought to have the sequences set forth in SEQ ID NOs: 7-12, particularly SEQ ID NOs: 7-9.

SEQ ID NOs: 1-12 relate to sequences deposited under the following NCBI or Ensembl accession numbers: NR_024206.2, NR_024204.2, NR_024205.3, NR_146460.1, NR_146461.1, ENST00000331944.10, NR_028589.1, NR_028590.1, NR_028591.1, ENSMUST00000135433.8, ENSMUST00000138486.2 and ENSMUST00000151427.2, respectively. Orthologues of the human and mouse sequences (i.e. structurally related RNAs from other species with the same function as human Cytor) have been identified in other animals, including sheep, pigs and horses, as set forth in SEQ ID NOs: 13-15, respectively. The skilled person readily could determine whether an RNA from another animal is an orthologue of the recited sequences using techniques well-known in the art.

The genomic sequence of human Cytor is provided set forth in SEQ ID NOs: 16 and 17, which represent the NCBI and Ensembl deposited sequences, respectively. The genomic sequence of mouse Cytor is provided set forth in SEQ ID NOs: 18 and 19, which represent the NCBI and Ensembl deposited sequences, respectively.

The cDNA sequences corresponding to SEQ ID NOs: 1-15 are provided as SEQ ID NOs: 20-34, respectively.

The term “Tead1” commonly designates “Tead 1 RNA” and “Tead 1 protein”. The Tead 1 protein designates TEA domain family member 1, which is also known as Transcriptional enhancer factor TEF-1 and transcription factor 13 (TCF-13) The Tead 1 protein is encoded by the TEAD1 gene. The Tead 1 RNA is the mRNA encoding the Ted1 protein.

The amino acid sequence of the Tead 1 protein is preferably as set forth in any one of SEQ ID NOs 43 to 46—or an orthologue or naturally-occurring variant thereof. Similarly, the nucleotide sequence of the Tead 1 cDNA is preferably as set forth in any one of SEQ ID NOs: 39 to 42—or an orthologue or naturally-occurring variant thereof. The Tead 1 mRNA is preferably as set forth in any one of SEQ ID NOs: 39 to 42, herein T is replaced by U. SEQ ID NOs 39/43, 40/44, 41/45 and 42/46 are from human, horse, mouse and pig, respectively.

The terms “Cytor agonist” or “Cytor RNA agonist” are used interchangeably herein and refer to agents capable of directly or indirectly increasing (promoting, enhancing or potentiating) the expression, activity or function of Cytor, e.g. increasing the amount of Cytor RNA in skeletal muscle.

The term “Tead1 antagonists” as used herein encompasses “Tead1 RNA antagonists” as well as “Tead1 protein antagonists” and refers to agents capable of directly or indirectly decreasing (limiting, reducing or diminishing) or abolishing the expression, activity or function of Tead1, e.g. decreasing the amount of Tead1 RNA and/or Tead1 protein in skeletal muscle.

Thus, in some embodiments, a Cytor agonist may be viewed as an agent that increases the amount of Cytor RNA and a Tead1 antagonist may be viewed as an agent that decreases the amount of Tead1 RNA and/or protein in skeletal muscle (e.g. in myoblasts in skeletal muscle). It will be evident that such agonists selectively increase the expression, activity or function of Cytor, e.g. selectively increase the amount of Cytor RNA in skeletal muscle. Similarly, such antagonists selectively decrease the expression, protein level, activity or function of Tead 1, e.g. selectively decrease the amount of Tead1 RNA and/or protein in skeletal muscle. Alternatively viewed, agents that result in a global increase in expression that indirectly result in an increase in Cytor expression, function or expression are not Cytor agonists according to the invention. Likewise, agents that result in a global decrease in expression or protein that indirectly result in a decrease in Tead1 expression, protein level, function or expression are not Tead1 antagonists according to the invention. However, it will be evident to the skilled person that an increase in Cytor expression, function or activity and/or a decrease in the Tead1 expression, protein level, activity or function of Tead1 will have downstream effects on the expression of other genes, e.g. myosin encoding genes. Thus, a Cytor agonist may result in an increase in the expression of Cytor and one or more other genes and/or a Tead1 agonist may result in a decrease in the expression of Tead1 and one or more other genes. For instance, a Cytor agonist that functions directly may interact directly with Cytor RNA to increase (promote, enhance or potentiate) the activity or function of Cytor. Whilst the mechanism of Cytor action has not been elucidated, it is thought that it may interact with various transcriptional regulators and factors associated with chromatin assembly and packaging. Accordingly, Cytor may interact directly with one or more target proteins and/or non-coding RNAs, e.g. microRNAs. Thus, a Cytor agonist may function by increasing (promoting, enhancing or potentiating) the interaction between Cytor and one or more of its target molecules, e.g. one or more proteins and/or non-coding RNAs.

As a Cytor agonist may be viewed as an agent that increases the amount of Cytor RNA in skeletal muscle, in some embodiments the Cytor agonist may be Cytor RNA or an orthologue thereof or a functionally equivalent fragment and/or variant of said Cytor RNA or orthologue. In other words, a Cytor agonist may be a Cytor RNA administered to a subject in a sufficient amount (e.g. an effective amount) to increase the amount of Cytor RNA in skeletal muscle, e.g. compared to the amount of Cytor RNA in skeletal muscle prior to administration of the agonist.

Where the Cytor agonist is Cytor RNA, it may be preferred that an RNA molecule having the nucleotide sequence of the endogenous RNA is used, i.e. where the subject to be treated is human, a human Cytor RNA or functionally equivalent variant thereof is used (e.g. an endogenous variant, such as a splice variant). However, as shown in the Examples, human Cytor RNA has been shown to promote myogenesis in non-human species. Thus, in some embodiments, the Cytor RNA may have a nucleotide sequence from a different species or may be a synthetic Cytor RNA, e.g. a non-naturally occurring variant or fragment of a Cytor RNA.

In some embodiments, the Cytor agonist comprises RNA comprising a nucleotide sequence as set forth in any one of SEQ ID NOs:1-15 (e.g. any one of SEQ ID NOs: 1-6, preferably SEQ ID NO: 1 or 6) or a nucleotide sequence with at least 80% sequence identity to a sequence as set forth in any one of SEQ ID NOs: 1-15 (e.g. any one of SEQ ID NOs: 1-6, preferably SEQ ID NO: 1 or 6) or a functional portion (fragment) of any of said sequences, wherein the RNA is functionally equivalent to one or more of the recited sequences (e.g. a RNA with a nucleotide sequence of one of SEQ ID NOs: 1-6, preferably SEQ ID NO: 1 or 6) as defined below, e.g. promotes myogenesis in skeletal muscle.

Preferably, the nucleic acid molecule above is at least 85, 90, 95, 96, 97, 98, 99 or 100% identical to the sequence to which it is compared.

Nucleic acid sequence identity may be determined by, e.g. FASTA Search using GCG packages, with default values and a variable pamfactor, and gap creation penalty set at 12.0 and gap extension penalty set at 4.0 with a window of 6 nucleotides. Preferably said comparison is made over the full length of the sequence, but may be made over a smaller window of comparison, e.g. less than 300, 200, 100 or 50 contiguous nucleotides.

The Cytor RNA may be administered using any suitable means known in the art, as discussed in more detail below. In some embodiments, the RNA is provided in a nanoparticle. Thus, in some embodiments, the Cytor agonist may be viewed as an agent that increases the amount of Cytor RNA in skeletal muscle, wherein the agent is a nanoparticle comprising Cytor RNA. Any suitable nanoparticle may be used. In some embodiments, the nanoparticle is a lipid-based nanoparticle, e.g. a liposome, such as an immunoliposome. The nanoparticle comprising the Cytor RNA may be provided in a pharmaceutical composition as defined further below.

A Cytor agonist that functions indirectly may interact indirectly with Cytor RNA to selectively increase (promote, enhance or potentiate) the activity or function of Cytor. For instance, a Cytor agonist may interact directly with one or more of Cytor's target molecules, e.g. proteins and/or non-coding RNAs, e.g. microRNAs, such that an increase in Cytor activity is observed. Thus, a Cytor agonist may function indirectly by increasing (promoting, enhancing or potentiating) the interaction between Cytor and one or more of its target molecules, e.g. one or more proteins and/or non-coding RNAs.

As a Cytor agonist may be viewed as an agent that increases the amount of Cytor RNA in skeletal muscle, in some embodiments the Cytor agonist may function by inducing expression of Cytor RNA in skeletal muscle. For instance, a Cytor agonist may interact with one or more transcriptional regulators involved in controlling the expression of Cytor, e.g. one or more transcriptional regulators (e.g. a protein, such as a transcription factor), that interact with the Cytor promoter or an enhancer (or the transcriptional complex bound to the Cytor promoter or an enhancer) to selectively increase the amount of Cytor RNA in skeletal muscle. It will be evident that a Cytor agonist may function by inhibiting (e.g. reducing or blocking) the activity or function of a transcriptional regulator that suppresses Cytor expression. Alternatively, a Cytor agonist may function by increasing (e.g. promoting, enhancing or potentiating) the function of a transcriptional regulator that induces Cytor expression.

In some embodiments, the Cytor agonist may be an agent that selectively induces endogenous Cytor expression by interacting with the Cytor promoter or an enhancer (or the transcriptional complex bound to the Cytor promoter or an enhancer) directly. For instance, the Cytor agonist may comprise a polypeptide comprising a domain that binds to the Cytor promoter that is operably linked to a domain comprising a transcriptional activator.

In a representative example, the domain that binds to the Cytor promoter may be a CRISPR associated protein (e.g. a nuclease-deficient CRISPR associated protein, e.g. spdCas9 or sadCas9). Accordingly, the Cytor agonist (i.e. agent that increases or induces Cytor expression in skeletal muscle) may further comprise a guide RNA capable of hydridising with the Cytor promoter nucleic acid.

The skilled person readily could design a suitable guide RNA based on the known sequence of the Cytor promoter and well-established guidance in the art for designing such molecules. However, in a representative embodiment, a guide RNA may comprise or consist of a nucleotide sequence as set forth in SEQ ID NO: 35 or 36. In some embodiments, the guide RNA may comprise additional sequences, such as a capture sequence that binds to a trans-enhancer nucleic acid molecule.

Any suitable domain comprising a transcriptional activator may be used and the skilled person could select an appropriate domain based on known polypeptides with the required activity. In a representative example, a domain comprising a transcriptional activator may be derived from VP64, MS2, P65 or HSF1.

The skilled person readily would understand that the polypeptide defined above may be provided by administering one or more nucleic acid molecules encoding the polypeptide and optionally the guide RNA (and any other necessary nucleic acid molecule(s), e.g. a trans-enhancer nucleic acid), that is/are expressed in a cell (e.g. a myoblast cell), i.e. transcribed and/or translated in the cell.

Means for administering nucleic acid molecules are known in the art and any suitable means could be used to administer the nucleic acid molecules described herein. For instance, the nucleic acid molecules may be administered in the form of a nanoparticle. In some embodiments, the nanoparticle is a lipid-based nanoparticle, e.g. a liposome, such as an immunoliposome. In some embodiments, the nanoparticle may be a viral nanoparticle. Thus, in some embodiments, the Cytor agonist may be viewed as an agent that selectively increases the amount of Cytor RNA in skeletal muscle, wherein the agent is a viral nanoparticle or viral vector comprising a nucleic acid molecule Cytor agonist (e.g. that induces the expression of endogenous RNA). The nanoparticle comprising the nucleic acid molecule Cytor agonist may be provided in a pharmaceutical composition as defined further below.

Thus, viewed more generally, in some embodiments the Cytor agonist or agent that selectively increases or induces Cytor RNA in skeletal muscle comprises a gene therapy, i.e. the administration of one or more nucleic acid molecules that function to selectively increase the amount of Cytor RNA in skeletal muscle.

It will be evident the numerous gene therapy strategies may be employed to increase or induce Cytor RNA in skeletal muscle using techniques that are known in the art. In some embodiments, the gene therapy may involve the integration of nucleic acid molecules into the genome of one or more cells, e.g. myoblast cells, or a modification (e.g. substitution of one or more nucleotides, such as substitution of the G nucleotide with the A nucleotide at the cis-eQTL rs74360724 or such as substitution of the T nucleotide with the C nucleotide at the cis-eQTL rs such as substitution of the G nucleotide with the A nucleotide at the cis-eQTL rs74360724) of the genome of said cells to effect a permanent change in Cytor expression. In some embodiments, the gene therapy may involve the administration of nucleic acid molecules that do not integrate into the genome of cells and result only in a transient change in the amount of Cytor RNA, e.g. a transient change in endogenous Cytor expression.

In some embodiments, the gene therapy may involve the administration of a nucleic acid molecule encoding Cytor RNA or an orthologue thereof or a functionally equivalent fragment or variant of said Cytor RNA or orthologue. The nucleic acid molecule may be transcribed by cells in the skeletal muscle (e.g. myoblasts) thereby resulting in an increase of Cytor RNA in the skeletal muscle.

Thus, in some embodiments, the Cytor agonist comprises a nucleic acid molecule (e.g. DNA) encoding Cytor RNA comprising a nucleotide sequence as set forth in any one of SEQ ID NOs:1-15 (e.g. any one of SEQ ID NOs: 1-6, preferably SEQ ID NO: 1 or 6) or a nucleotide sequence with at least 80% sequence identity to a sequence as set forth in any one of SEQ ID NOs: 1-15 (e.g. any one of SEQ ID NOs: 1-6, preferably SEQ ID NO: 1 or 6) or a functional portion (fragment) of any of said sequences, wherein the RNA is functionally equivalent to one or more of the recited sequences (e.g. a RNA with a nucleotide sequence of one of SEQ ID NOs: 1-6, preferably SEQ ID NO: 1 or 6) as defined below, e.g. promotes myogenesis in skeletal muscle.

Alternatively viewed, in some embodiments, the Cytor agonist comprises a nucleic acid molecule (e.g. DNA) encoding Cytor RNA, wherein the nucleic acid molecule comprises a nucleotide sequence as set forth in any one of SEQ ID NOs:20-34 (e.g. any one of SEQ ID NOs: 20-25, preferably SEQ ID NO: 20 or 25) or a nucleotide sequence with at least 80% sequence identity to a sequence as set forth in any one of SEQ ID NOs: 20-34 (e.g. any one of SEQ ID NOs: 20-25, preferably SEQ ID NO: 20 or 25) or a functional portion (fragment) or complement of any of said sequences, wherein the RNA encoded by the nucleic acid molecule is functionally equivalent to one or more of an RNA comprising a sequence as set forth in any one of SEQ ID NOs: 1-15 (e.g. a RNA with a nucleotide sequence of one of SEQ ID NOs: 1-6, preferably SEQ ID NO: 1 or 6) as defined below, e.g. promotes myogenesis in skeletal muscle.

Preferably, the nucleic acid molecule above is at least 85, 90, 95, 96, 97, 98, 99 or 100% identical to the sequence to which it is compared. Nucleic acid sequence identity may be determined as defined above.

In some embodiments, the gene therapy may involve the administration of a nucleic acid molecule encoding a polypeptide that functions to increase the amount of Cytor RNA in skeletal muscle. As noted above, the polypeptide may be a synthetic polypeptide (e.g. fusion protein) that interacts with the Cytor promoter to induce Cytor expression. However, the nucleic acid molecule may encode a polypeptide that functions as a Cytor agonist as described above, e.g. that interacts with one or more of Cytor's target molecule or that interacts with one or more transcriptional regulators of Cytor expression. In a further representative embodiment, the nucleic acid molecule may encode a polypeptide that inhibits the degradation of endogenous Cytor, e.g. an RNA binding protein.

In some embodiments, the nucleic acid molecule(s) encoding the polypeptide may be provided as RNA, such that it can be translated to provide the polypeptide directly.

In some embodiments, the nucleic acid molecule(s) encoding the polypeptide or Cytor RNA may be provided in a form that requires processing by the cell to produce the polypeptide or Cytor RNA, e.g. the nucleic acid molecule(s) may be DNA. Suitably, the nucleic acid molecule(s) may comprise a control or regulatory sequence (e.g. promoter sequence) suitable for transcription of the encoding sequence in muscle, e.g. skeletal muscle. Thus, the nucleic acid molecule(s) encoding the polypeptide or Cytor RNA may be operably linked to a regulatory sequence. In some embodiments, the regulatory sequence is tissue-specific promoter, e.g. a muscle-specific promoter, such as a promoter that functions only in skeletal muscle, e.g. a myoblast-specific promoter. A suitable promoter may be the muscle creatine kinase (MCK) promoter or a variant thereof, e.g. a sequence deposited under Genbank AF188002.1 or functional variant or fragment thereof. Thus, in some embodiments, the regulatory sequence (promoter) comprises a nucleotide sequence as set forth in SEQ ID NO: 37 or a nucleotide sequence with at least 80% sequence identity to a sequence as set forth in SEQ ID NO: 37 or a functional portion (fragment) or complement of any of said sequences, wherein the sequence functions to enable expression of the nucleic acid molecule in muscle tissue (i.e. to limit expression of the nucleic acid molecule to muscle tissue).

A Tead1 antagonist may be viewed as an agent that decreases the amount of Tead1 RNA and/or protein in skeletal muscle. For instance, a Tead1 antagonist that functions directly may interact directly with Tead1 RNA and/or protein to decrease (inhibit, reduce or silence) the activity or function of Tead1 RNA and/or Tead1 protein.

In accordance with a preferred embodiment the antagonist inhibits the expression and/or the activity of Tead1.

A compound inhibiting the expression of Tead1 is in accordance with the present invention a compound lowering or preventing the transcription of the gene encoding the Tead1 and/or inhibiting the translation of the Tead1 mRNA into the Tead1 protein. Such compounds include compounds interfering with the transcriptional machinery and/or its interaction with the promoter of said gene and/or with expression control elements remote from the promoter such as enhancers. Such compounds also include compounds interfering with the translational machinery. The compound inhibiting the expression of Tead1 specifically inhibits the expression of Tead1, for example, by specifically interfering with the promoter region controlling the expression of Tead1. Preferably, the transcription and/or translation of Tead1 is reduced by at least 50%, more preferred at least 75% such as at least 90% or 95%, even more preferred at least 98% and most preferred by about 100% (e.g., as compared to the same experimental set up in the absence of the agent). Examples of such compounds are compounds selected from a small molecule, an aptamer, a siRNA, a shRNA, a miRNA, a ribozyme, an antisense nucleic acid molecule, a CRISPR-Cas9-based construct, a CRISPR-Cpf1-based construct, a meganuclease, a zinc finger nuclease, and a transcription activator-like (TAL) effector (TALE) nuclease

A compound inhibiting the activity of Tead1 in accordance with the present invention causes the Tead1 protein to perform its function with lowered efficiency. The compound inhibiting the activity of Tead1 protein specifically inhibits the activity of said protein. Preferably, the activity of the Tead1 protein is reduced by at least 50%, more preferred at least 75% such as at least 90% or 95%, even more preferred at least 98%, and most preferably about 100% (e.g., as compared to the same experimental set up in the absence of the compound). Tead1 is a transcription factor comprising a DNA binding domain called the TEA domain. The decrease in Tead1 activity is preferably a decrease of the TEA domain to bind their target DNA sites. Means and methods for determining the reduction of activity of a protein are established in the art. Examples of such compounds are compounds are an antibody, an antibody mimetic and an aptamer, wherein the antibody mimetic is preferably selected from affibodies, adnectins, anticalins, DARPins, avimers, nanofitins, affilins, Kunitz domain peptides, Fynomers, trispecific binding molecules and probodies.

In accordance with another preferred embodiment, the Tead1 antagonist is a small molecule, a nucleotide-based inhibitor or an amino acid-based inhibitor.

In accordance with a more preferred embodiment the nucleotide-based inhibitor is an aptamer, a ribozyme, a siRNA, a shRNA or an antisense oligonucleotide and the amino acid-based inhibitor is an antibody or an antibody mimetic.

The “small molecule” as used in this preferred embodiment is preferably an organic molecule. Organic molecules relate or belong to the class of chemical compounds having a carbon basis, the carbon atoms linked together by carbon-carbon bonds. The original definition of the term organic related to the source of chemical compounds, with organic compounds being those carbon-containing compounds obtained from plant or animal or microbial sources, whereas inorganic compounds were obtained from mineral sources. Organic compounds can be natural or synthetic. The organic molecule is preferably an aromatic molecule and more preferably a heteroaromatic molecule. In organic chemistry, the term aromaticity is used to describe a cyclic (ring-shaped), planar (flat) molecule with a ring of resonance bonds that exhibits more stability than other geometric or connective arrangements with the same set of atoms. Aromatic molecules are very stable, and do not break apart easily to react with other substances. In a heteroaromatic molecule at least one of the atoms in the aromatic ring is an atom other than carbon, e.g. N, S, or O. For all above-described organic molecules the molecular weight is preferably in the range of 200 Da to 1500 Da and more preferably in the range of 300 Da to 1000 Da.

The small molecule is preferably a proteolysis targeting chimera (PROTAC)-A PROTAC is a heterobifunctional small molecule composed of two active domains and a linker, capable of removing specific unwanted proteins. Rather than acting as a conventional enzyme inhibitor, a PROTAC works by inducing selective intracellular proteolysis. PROTACs consist of two covalently linked protein-binding molecules: one capable of engaging an E3 ubiquitin ligase, and another that binds to a target protein meant for degradation. Recruitment of the E3 ligase to the target protein results in ubiquitination and subsequent degradation of the target protein via the proteasome. Because PROTACs need only to bind their targets with high selectivity (rather than inhibit the target protein's enzymatic activity), previously ineffective inhibitor molecules that only provide for selective binding but no or insufficient inhibition may be further used in order to develop PROTACs.

Alternatively, the “small molecule” may be an inorganic compound. Inorganic compounds are derived from mineral sources and include all compounds without carbon atoms (except carbon dioxide, carbon monoxide and carbonates). Preferably, the small molecule has a molecular weight of less than about 2000 Da, or less than about 1000 Da such as less than about 500 Da, and even more preferably less than about Da amu. The size of a small molecule can be determined by methods well-known in the art, e.g., mass spectrometry. The small molecules may be designed, for example, based on the crystal structure of the target molecule, where sites presumably responsible for the biological activity can be identified and verified in in vivo assays such as in vivo high-throughput screening (HTS) assays.

A nucleotide-based inhibitor comprises or consists of a nucleic acid sequence. The nucleic acid is preferably complementary to a nucleic acid sequence of at least 12 contiguous nucleotides of one or more of SEQ ID NOs 39 to 42. The nucleotide-based inhibitor may comprise or consist of RNA, DNA or both. The nucleotide-based inhibitor of the invention is a molecule that binds specifically to Tead1 RNA. As used herein specific binding means that the inhibitor specifically targets the Tead1 mRNA and does substantially not exert any off target inhibitory effects, in particular on other cellular nucleic acid molecules.

An amino acid-based inhibitor comprises or consists of an amino acid sequence and preferably an amino acid sequence of at least 25, more preferably at least 50 amino acids. The amino acid-based inhibitor of the invention is a molecule that binds specifically to the Tead1 protein and in addition inhibits the activity of Tead1. The amino acid-based inhibitor preferably comprises natural amino acids but may also comprise unnatural amino acids. The amino acid-based inhibitor is preferably selected or designed such that it specifically binds to an amino acid sequence selected from one or more of SEQ ID NOs 43 to 46.

The term “antibody” as used in accordance with the present invention comprises, for example, polyclonal or monoclonal antibodies. Furthermore, also derivatives or fragments thereof, which still retain the binding specificity to the target, e.g. Tead1, are comprised in the term “antibody”. Antibody fragments or derivatives comprise, inter alia, Fab or Fab′ fragments, Fd, F(ab′)2, Fv or scFv fragments, single domain VH or V-like domains, such as VhH or V-NAR-domains, as well as multimeric formats such as minibodies, diabodies, tribodies or triplebodies, tetrabodies or chemically conjugated Fab′-multimers (see, for example, Harlow and Lane “Antibodies, A Laboratory Manual”, Cold Spring Harbor Laboratory Press, 198; Harlow and Lane “Using Antibodies: A Laboratory Manual” Cold Spring Harbor Laboratory Press, 1999; Altshuler E P, Serebryanaya D V, Katrukha A G. 2010, Biochemistry (Mosc)., vol. 75(13), 1584; Holliger P, Hudson P J. 2005, Nat Biotechnol., vol. 23(9), 1126). The multimeric formats in particular comprise bispecific antibodies that can simultaneously bind to two different types of antigen. The first antigen can be found on the protein of the invention. The second antigen may, for example, be a tumor marker that is specifically expressed on cancer cells or a certain type of cancer cells. Non-limiting examples of bispecific antibodies formats are Biclonics (bispecific, full length human IgG antibodies), DART (Dual-affinity Re-targeting Antibody) and BiTE (consisting of two single-chain variable fragments (scFvs) of different antibodies) molecules (Kontermann and Brinkmann (2015), Drug Discovery Today, 20(7):838-847).

The term “antibody” also includes embodiments such as chimeric (human constant domain, non-human variable domain), single chain and humanised (human antibody with the exception of non-human CDRs) antibodies.

Various techniques for the production of antibodies are well known in the art and described, e.g. in Harlow and Lane (1988) and (1999) and Altshuler et al., 2010, loc. cit. Thus, polyclonal antibodies can be obtained from the blood of an animal following immunisation with an antigen in mixture with additives and adjuvants and monoclonal antibodies can be produced by any technique which provides antibodies produced by continuous cell line cultures. Examples for such techniques are described, e.g. in Harlow E and Lane D, Cold Spring Harbor Laboratory Press, 1988; Harlow E and Lane D, Using Antibodies: A Laboratory Manual, Cold Spring Harbor Laboratory Press, 1999 and include the hybridoma technique originally described by Köhler and Milstein, 1975, the trioma technique, the human B-cell hybridoma technique (see e.g. Kozbor D, 1983, Immunology Today, vol. 4, 7; Li J, et al. 2006, PNAS, vol. 103(10), 3557) and the EBV-hybridoma technique to produce human monoclonal antibodies (Cole et al., 1985, Alan R. Liss, Inc, 77-96). Furthermore, recombinant antibodies may be obtained from monoclonal antibodies or can be prepared de novo using various display methods such as phage, ribosomal, mRNA, or cell display. A suitable system for the expression of the recombinant (humanised) antibodies may be selected from, for example, bacteria, yeast, insects, mammalian cell lines or transgenic animals or plants (see, e.g., U.S. Pat. No. 6,080,560; Holliger P, Hudson P J. 2005, Nat Biotechnol., vol. 23(9), 11265). Further, techniques described for the production of single chain antibodies (see, inter alia, U.S. Pat. No. 4,946,778) can be adapted to produce single chain antibodies specific for an epitope of Tead1. Surface plasmon resonance as employed in the BIAcore system can be used to increase the efficiency of phage antibodies.

As used herein, the term “antibody mimetics” refers to compounds which, like antibodies, can specifically bind antigens, such as Tead1, but which are not structurally related to antibodies. Antibody mimetics are usually artificial peptides or proteins with a molar mass of about 3 to 20 kDa. For example, an antibody mimetic may be selected from the group consisting of affibodies, adnectins, anticalins, DARPins, avimers, nanofitins, affilins, Kunitz domain peptides, Fynomers, trispecific binding molecules and prododies. These polypeptides are well known in the art and are described in further detail herein below.

The term “affibody”, as used herein, refers to a family of antibody mimetics which is derived from the Z-domain of staphylococcal protein A. Structurally, affibody molecules are based on a three-helix bundle domain which can also be incorporated into fusion proteins. In itself, an affibody has a molecular mass of around 6 kDa and is stable at high temperatures and under acidic or alkaline conditions. Target specificity is obtained by randomisation of 13 amino acids located in two alpha-helices involved in the binding activity of the parent protein domain (Feldwisch J, Tolmachev V.; (2012) Methods Mol Biol. 899:103-26).

The term “adnectin” (also referred to as “monobody”), as used herein, relates to a molecule based on the 10th extracellular domain of human fibronectin III (10Fn3), which adopts an Ig-like β-sandwich fold of 94 residues with 2 to 3 exposed loops, but lacks the central disulphide bridge (Gebauer and Skerra (2009) Curr Opinion in Chemical Biology 13:245-255). Adnectins with the desired target specificity, i.e. against Tead1, can be genetically engineered by introducing modifications in specific loops of the protein.

The term “anticalin”, as used herein, refers to an engineered protein derived from a lipocalin (Beste G, Schmidt F S, Stibora T, Skerra A. (1999) Proc Natl Acad Sci USA. 96(5):1898-903; Gebauer and Skerra (2009) Curr Opinion in Chemical Biology 13:245-255). Anticalins possess an eight-stranded β-barrel which forms a highly conserved core unit among the lipocalins and naturally forms binding sites for ligands by means of four structurally variable loops at the open end. Anticalins, although not homologous to the IgG superfamily, show features that so far have been considered typical for the binding sites of antibodies: (i) high structural plasticity as a consequence of sequence variation and (ii) elevated conformational flexibility, allowing induced fit to targets with differing shape.

As used herein, the term “DARPin” refers to a designed ankyrin repeat domain (166 residues), which provides a rigid interface arising from typically three repeated β-turns. DARPins usually carry three repeats corresponding to an artificial consensus sequence, wherein six positions per repeat are randomised. Consequently, DARPins lack structural flexibility (Gebauer and Skerra, 2009).

The term “avimer”, as used herein, refers to a class of antibody mimetics which consist of two or more peptide sequences of 30 to 35 amino acids each, which are derived from A-domains of various membrane receptors and which are connected by linker peptides. Binding of target molecules occurs via the A-domain and domains with the desired binding specificity, i.e. for Tead1, can be selected, for example, by phage display techniques. The binding specificity of the different A-domains contained in an avimer may, but does not have to be identical (Weidle U H, et al., (2013), Cancer Genomics Proteomics; 10(4):155-68).

A “nanofitin” (also known as affitin) is an antibody mimetic protein that is derived from the DNA binding protein Sac7d of Sulfolobus acidocaldarius. Nanofitins usually have a molecular weight of around 7 kDa and are designed to specifically bind a target molecule, such as e.g. Tead1, by randomising the amino acids on the binding surface (Mouratou B, Béhar G, Paillard-Laurance L, Colinet S, Pecorari F., (2012) Methods Mol Biol.; 805:315-31).

The term “affilin”, as used herein, refers to antibody mimetics that are developed by using either gamma-B crystalline or ubiquitin as a scaffold and modifying amino-acids on the surface of these proteins by random mutagenesis. Selection of affilins with the desired target specificity, i.e. against Tead1, is effected, for example, by phage display or ribosome display techniques. Depending on the scaffold, affilins have a molecular weight of approximately 10 or 20 kDa. As used herein, the term affilin also refers to di- or multimerised forms of affilins (Weidle U H, et al., (2013), Cancer Genomics Proteomics; 10(4):155-68).

A “Kunitz domain peptide” is derived from the Kunitz domain of a Kunitz-type protease inhibitor such as bovine pancreatic trypsin inhibitor (BPTI), amyloid precursor protein (APP) or tissue factor pathway inhibitor (TFPI). Kunitz domains have a molecular weight of approximately 6 kDA and domains with the required target specificity, i.e. against Tead1, can be selected by display techniques such as phage display (Weidle et al., (2013), Cancer Genomics Proteomics; 10(4):155-68).

As used herein, the term “Fynomer” refers to a non-immunoglobulin-derived binding polypeptide derived from the human Fyn SH3 domain. Fyn SH3-derived polypeptides are well-known in the art and have been described e.g. in Grabulovski et al. (2007) JBC, 282, p. 3196-3204, WO 2008/022759, Bertschinger et al (2007) Protein Eng Des Sel 20(2):57-68, Gebauer and Skerra (2009) Curr Opinion in Chemical Biology 13:245-255, or Schlatter et al. (2012), MAbs 4:4, 1-12).

The term “trispecific binding molecule” as used herein refers to a polypeptide molecule that possesses three binding domains and is thus capable of binding, preferably specifically binding to three different epitopes. At least one of these three epitopes is an epitope of the protein of the fourth aspect of the invention. The two other epitopes may also be epitopes of the protein of the fourth aspect of the invention or may be epitopes of one or two different antigens. The trispecific binding molecule is preferably a TriTac. A TriTac is a T-cell engager for solid tumors which comprised of three binding domains being designed to have an extended serum half-life and be about one-third the size of a monoclonal antibody.

As used herein, the term “probody” refers to a protease-activatable antibody prodrug. A probody consists of an authentic IgG heavy chain and a modified light chain. A masking peptide is fused to the light chain through a peptide linker that is cleavable by tumor-specific proteases. The masking peptide prevents the probody binding to healthy tissues, thereby minimizing toxic side effects.

Aptamers are nucleic acid molecules or peptide molecules that bind a specific target molecule. Aptamers are usually created by selecting them from a large random sequence pool, but natural aptamers also exist in riboswitches. Aptamers can be used for both basic research and clinical purposes as macromolecular drugs. Aptamers can be combined with ribozymes to self-cleave in the presence of their target molecule. These compound molecules have additional research, industrial and clinical applications (Osborne et. al. (1997), Current Opinion in Chemical Biology, 1:5-9; Stull & Szoka (1995), Pharmaceutical Research, 12, 4:465-483).

Nucleic acid aptamers are nucleic acid species that normally consist of (usually short) strands of oligonucleotides. Typically, they have been engineered through repeated rounds of in vitro selection or equivalently, SELEX (systematic evolution of ligands by exponential enrichment) to bind to various molecular targets such as small molecules, proteins, nucleic acids, and even cells, tissues and organisms.

Peptide aptamers are usually peptides or proteins that are designed to interfere with other protein interactions inside cells. They consist of a variable peptide loop attached at both ends to a protein scaffold. This double structural constraint greatly increases the binding affinity of the peptide aptamer to levels comparable to an antibody's (nanomolar range). The variable peptide loop typically comprises 10 to 20 amino acids, and the scaffold may be any protein having good solubility properties. Currently, the bacterial protein Thioredoxin-A is the most commonly used scaffold protein, the variable peptide loop being inserted within the redox-active site, which is a -Cys-Gly-Pro-Cys-loop (SEQ ID NO: 55) in the wild protein, the two cysteins lateral chains being able to form a disulfide bridge. Peptide aptamer selection can be made using different systems, but the most widely used is currently the yeast two-hybrid system.

Aptamers offer the utility for biotechnological and therapeutic applications as they offer molecular recognition properties that rival those of the commonly used biomolecules, in particular antibodies. In addition to their discriminatory recognition, aptamers offer advantages over antibodies as they can be engineered completely in a test tube, are readily produced by chemical synthesis, possess desirable storage properties, and elicit little or no immunogenicity in therapeutic applications. Non-modified aptamers are cleared rapidly from the bloodstream, with a half-life of minutes to hours, mainly due to nuclease degradation and clearance from the body by the kidneys, a result of the aptamers' inherently low molecular weight. Unmodified aptamer applications currently focus on treating transient conditions such as blood clotting, or treating organs such as the eye where local delivery is possible. This rapid clearance can be an advantage in applications such as in vivo diagnostic imaging. Several modifications, such as 2′-fluorine-substituted pyrimidines, polyethylene glycol (PEG) linkage, fusion to albumin or other half life extending proteins etc. are available to scientists such that the half-life of aptamers can be increased for several days or even weeks.

In accordance with the present invention, the term “small interfering RNA (siRNA)”, also known as short interfering RNA or silencing RNA, refers to a class of 18 to 30, preferably 19 to 25, most preferred 21 to 23 or even more preferably 21 nucleotide-long double-stranded RNA molecules that play a variety of roles in biology. Most notably, siRNA is involved in the RNA interference (RNAi) pathway where the siRNA interferes with the expression of a specific gene. In addition to their role in the RNAi pathway, siRNAs also act in RNAi-related pathways, e.g. as an antiviral mechanism or in shaping the chromatin structure of a genome.

siRNAs naturally found in nature have a well defined structure: a short double-strand of RNA (dsRNA) with 2-nt 3′ overhangs on either end. Each strand has a 5′ phosphate group and a 3′ hydroxyl (—OH) group. This structure is the result of processing by dicer, an enzyme that converts either long dsRNAs or small hairpin RNAs into siRNAs. siRNAs can also be exogenously (artificially) introduced into cells to bring about the specific knockdown of a gene of interest. Essentially any gene for which the sequence is known can thus be targeted based on sequence complementarity with an appropriately tailored siRNA. The double-stranded RNA molecule or a metabolic processing product thereof is capable of mediating target-specific nucleic acid modifications, particularly RNA interference and/or DNA methylation. Exogenously introduced siRNAs may be devoid of overhangs at their 3′ and 5′ ends, however, it is preferred that at least one RNA strand has a 5′- and/or 3′-overhang. Preferably, one end of the double-strand has a 3′-overhang from 1 to 5 nucleotides, more preferably from 1 to 3 nucleotides and most preferably 2 nucleotides. The other end may be blunt-ended or has up to 6 nucleotides 3′-overhang. In general, any RNA molecule suitable to act as siRNA is envisioned in the present invention. The most efficient silencing was so far obtained with siRNA duplexes composed of 21-nt sense and 21-nt antisense strands, paired in a manner to have a 2-nt 3′-overhang. The sequence of the 2-nt 3′ overhang makes a small contribution to the specificity of target recognition restricted to the unpaired nucleotide adjacent to the first base pair (Elbashir et al. 2001). 2′-deoxynucleotides in the 3′ overhangs are as efficient as ribonucleotides, but are often cheaper to synthesize and probably more nuclease resistant. Delivery of siRNA may be accomplished using any of the methods known in the art, for example by combining the siRNA with saline and administering the combination intravenously or intranasally or by formulating siRNA in glucose (such as for example 5% glucose) or cationic lipids and polymers can be used for siRNA delivery in vivo through systemic routes either intravenously (IV) or intraperitoneally (IP) (Fougerolles et al. (2008), Current Opinion in Pharmacology, 8:280-285; Lu et al. (2008), Methods in Molecular Biology, vol. 437: Drug Delivery Systems—Chapter 3: Delivering Small Interfering RNA for Novel Therapeutics).

A short hairpin RNA (shRNA) is a sequence of RNA that makes a tight hairpin turn that can be used to silence gene expression via RNA interference. shRNA uses a vector introduced into cells and utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs which match the siRNA that is bound to it. si/shRNAs to be used in the present invention are preferably chemically synthesized using appropriately protected ribonucleoside phosphoramidites and a conventional DNA/RNA synthesizer. Suppliers of RNA synthesis reagents are Proligo (Hamburg, Germany), Dharmacon Research (Lafayette, CO, USA), Pierce Chemical (part of Perbio Science, Rockford, IL, USA), Glen Research (Sterling, VA, USA), ChemGenes (Ashland, MA, USA), and Cruachem (Glasgow, UK). Most conveniently, siRNAs or shRNAs are obtained from commercial RNA oligo synthesis suppliers, which sell RNA-synthesis products of different quality and costs. In general, the RNAs applicable in the present invention are conventionally synthesized and are readily provided in a quality suitable for RNAi.

Further molecules effecting RNAi include, for example, microRNAs (miRNA). Said RNA species are single-stranded RNA molecules. Endogenously present miRNA molecules regulate gene expression by binding to a complementary mRNA transcript and triggering of the degradation of said mRNA transcript through a process similar to RNA interference. Accordingly, exogenous miRNA may be employed as an inhibitor of Tead1 after introduction into the respective cells.

In accordance with a further preferred embodiment the nucleotide based inhibitor comprises (a) a nucleic acid sequence which comprises or consists of a nucleic acid sequence being complementary to at least 12 continuous nucleotides of a nucleic acid sequence selected from SEQ ID NOs 39 to 42, (b) a nucleic acid sequence which comprises or consists of a nucleic acid sequence which is at least 70% identical to the complementary strand of one or more nucleic acid sequences selected from SEQ ID NOs 39 to 42, (c) a nucleic acid sequence which comprises or consists of a nucleic acid sequence according to (a) or (b), wherein the nucleic acid sequence is DNA or RNA, or (d) an expression vector expressing the nucleic acid sequence as defined in any one of (a) to (c), preferably under the control of a skeletal muscle-specific promoter.

The nucleic acid sequences as defined in items (a) to (c) of this preferred embodiment comprise or consist of sequences being complementary to nucleotides of Tead1 as defined by one or more of SEQ ID NOs 39 to 43. Hence, the nucleic acid sequences as defined in items (a) to (c) comprise or are antisense nucleic acid sequences.

The nucleic acid sequence according to item (a) of this further preferred embodiment of the invention comprises or consists of a sequence which is with increasing preference complementary to at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides of one or more selected from SEQ ID NOs 39 to 42. The format of the nucleic acid sequence according to item (a) is not particularly limited as long as it comprises or consists of at least 12 continuous nucleotides being complementary to a nucleic acid sequence selected from SEQ ID NOs 39 to 42. The nucleic acid sequence according to item (a) comprises or consists of antisense an oligonucleotide. Hence, the nucleic acid sequence according to item (a) reflects the above-mentioned basic principle of the antisense technology which is the use of an oligonucleotide for silencing a selected target RNA through the exquisite specificity of complementary-based pairing. Therefore, it is to be understood that the nucleic acid sequence according to item (a) is preferably in the format of an siRNA, shRNA or an antisense oligonucleotide as defined herein above. The antisense oligonucleotides are preferably LNA-GapmeRs, AntagomiRs, or antimiRs.

The nucleic acid sequence according to item (b) requiring at least 70% identity to the complementary strand of one or more nucleic acid sequences selected from SEQ ID NOs 39 to is considerably longer than the nucleic acid sequence according to item (a) which comprises an antisense oligonucleotide and comprises at least 12 continuous nucleotides of a nucleic acid sequence selected from SEQ ID NOs 39 to. A nucleic acid sequence according to item (b) of the above preferred embodiment of the invention is capable of interacting with, more specifically hybridizing with the target Tead1 RNA. By formation of the hybrid the function of the translation of the Tead1 RNA is reduced or blocked.

The sequence identity of the molecule according to item (b) in connection with a sequence selected from SEQ ID NOs 39 to is with increasing preference at least 75%, at least 80%, at least 85%, at least 90%, at least 92.5%, at least 95%, at least 98%, at least 99% and 100%.

In the nucleic acid sequence according to item (c) the nucleotide sequences may be RNA or DNA. RNA or DNA encompasses chemically modified RNA nucleotides or DNA nucleotides. As commonly known RNA comprises the nucleotide U while DNA comprises the nucleotide T.

In accordance with items (d) and (e) of the above preferred embodiment the inhibitor may also be an expression vector, respectively being capable of expressing a nucleic acid sequence as defined in any one of items (a) to (c).

An expression vector may be a plasmid that is used to introduce a specific transcript into a target cell. Once the expression vector is inside the cell, the protein that is encoded by the gene is produced by the cellular-transcription and translation machinery ribosomal complexes. The plasmid is in general engineered to contain regulatory sequences that act as enhancer and/or promoter regions and lead to efficient transcription of the transcript. In accordance with the present invention the expression vector preferably contains a muscle-specific promoter, preferably skeletal muscle-specific promoter. Using such a promoter ensures that the nucleic acid sequence is only expressed in the (skeletal) muscle and may avoid potential unwanted side effects by expression in other organs. Such promoters are described, for example, in Skopenova (2021), Acta Naturae. 2021 January-March; 13(1): 47-58.

Non-limiting examples of expression vectors include prokaryotic plasmid vectors, such as the pUC-series, pBluescript (Stratagene), the pET-series of expression vectors (Novagen) or pCRTOPO (Invitrogen) and vectors compatible with an expression in mammalian cells like pREP (Invitrogen), pcDNA3 (Invitrogen), pCEP4 (Invitrogen), pMC1neo (Stratagene), pXT1 (Stratagene), pSG5 (Stratagene), EBO-pSV2neo, pBPV-1, pdBPVMMTneo, pRSVgpt, pRSVneo, pSV2-dhfr, plZD35, pLXIN, pSIR (Clontech), pIRES-EGFP (Clontech), pEAK-10 (Edge Biosystems) pTriEx-Hygro (Novagen) and pCINeo (Promega). Examples for plasmid vectors suitable for Pichia pastoris comprise e.g. the plasmids pAO815, pPIC9K and pPIC3.5K (all Intvitrogen). For the formulation of a pharmaceutical composition a suitable vector is selected in accordance with good manufacturing practice. Such vectors are known in the art, for example, from Ausubel et al, Hum Gene Ther. 2011 April; 22(4):489-97 or Allay et al., Hum Gene Ther. May 2011; 22(5): 595-604.

A typical mammalian expression vector contains the promoter element, which mediates the initiation of transcription of mRNA, the protein coding sequence, and signals required for the termination of transcription and polyadenylation of the transcript. Moreover, elements such as origin of replication, drug resistance gene, regulators (as part of an inducible promoter) may also be included. The lac promoter is a typical inducible promoter, useful for prokaryotic cells, which can be induced using the lactose analogue isopropylthiol-b-D-galactoside (“IPTG”). For recombinant expression and secretion, the polynucleotide of interest may be ligated between e.g. the PelB leader signal, which directs the recombinant protein in the periplasm and the gene III in a phagemid called pHEN4 (described in Ghahroudi et al, 1997, FEBS Letters 414:521-526). Additional elements might include enhancers, Kozak sequences and intervening sequences flanked by donor and acceptor sites for RNA splicing. Highly efficient transcription can be achieved with the early and late promoters from SV40, the long terminal repeats (LTRs) from retroviruses, e.g., RSV, HTLVI, HIVI, and the early promoter of the cytomegalovirus (CMV). However, cellular elements can also be used (e.g., the human actin promoter). Suitable expression vectors for use in practicing the present invention include, for example, vectors such as pSVL and pMSG (Pharmacia, Uppsala, Sweden), pRSVcat (ATCC 37152), pSV2dhfr (ATCC 37146) and pBC12MI (ATCC 67109). Alternatively, the recombinant (poly)peptide can be expressed in stable cell lines that contain the gene construct integrated into a chromosome. The co-transfection with a selectable marker such as dhfr, gpt, neomycin, hygromycin allows the identification and isolation of the transfected cells. The transfected nucleic acid can also be amplified to express large amounts of the encoded (poly)peptide. The DHFR (dihydrofolate reductase) marker is useful to develop cell lines that carry several hundred or even several thousand copies of the gene of interest. Another useful selection marker is the enzyme glutamine synthase (GS) (Murphy et al. 1991, Biochem J. 227:277-279; Bebbington et al. 1992, Bio/Technology 10:169-175). Using these markers, the mammalian cells are grown in selective medium and the cells with the highest resistance are selected. As indicated above, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. For vector modification techniques, see Sambrook and Russel (2001), Molecular Cloning: A Laboratory Manual, 3 Vol. Generally, vectors can contain one or more origins of replication (ori) and inheritance systems for cloning or expression, one or more markers for selection in the host, e.g., antibiotic resistance, and one or more expression cassettes. Suitable origins of replication (ori) include, for example, the Col E1, the SV40 viral and the M 13 origins of replication.

The sequences to be inserted into the vector can e.g. be synthesized by standard methods, or isolated from natural sources. Ligation of the coding sequences to transcriptional regulatory elements and/or to other amino acid encoding sequences can be carried out using established methods. Transcriptional regulatory elements (parts of an expression cassette) ensuring expression in prokaryotes or eukaryotic cells are well known to those skilled in the art. These elements comprise regulatory sequences ensuring the initiation of the transcription (e.g., translation initiation codon, promoters, enhancers, and/or insulators), internal ribosomal entry sites (IRES) (Owens, Proc. Natl. Acad. Sci. USA 98 (2001), 1471-1476) and optionally poly-A signals ensuring termination of transcription and stabilization of the transcript. Additional regulatory elements may include transcriptional as well as translational enhancers, and/or naturally-associated or heterologous promoter regions. Preferably, the nucleotide sequence as defined in item (a) of the above preferred embodiment of the invention is operatively linked to such expression control sequences allowing expression in prokaryotic or eukaryotic cells.

The Tead 1 antagonist may be administered using any suitable means known in the art, as discussed in more detail below. In some embodiments, the nucleotide-based inhibitor is provided in a nanoparticle. Thus, in some embodiments, the nucleotide-based inhibitor may be viewed as an agent that decreases the amount of Cytor RNA in skeletal muscle, wherein the agent is a nanoparticle comprising a nucleotide-based inhibitor as defined. Any suitable nanoparticle may be used. In some embodiments, the nanoparticle is a lipid-based nanoparticle, e.g. a liposome, such as an immunoliposome. The nanoparticle comprising the nucleotide-based inhibitor may be provided in a pharmaceutical composition as defined further below.

A Tead1 antagonist that functions indirectly may interact indirectly with Tead1 RNA to selectively decrease (inhibit, reduce or silence) the activity or function of Tead1. For instance, a Tead1 antagonist agonist may interact directly with one or more of Tead1 's target molecules, e.g. proteins and/or non-coding RNAs, e.g. microRNAs, such that a decrease in Tead1 activity is observed. Thus, a Tead1 antagonist may function indirectly by decreasing (inhibiting, reducing or silencing) the interaction between Tead1 and one or more of its target molecules, noting that Tead1 is a transcription factor comprising DNA binding domain called the TEA domain. The decrease in Tead1 activity is preferably a decrease of the TEA domain to bind their target DNA sites. The decrease is with increasing preference at least 10%, at least 20%, at least 30%, at least 40%, at least 50%, at least 60%, at least 70%, at least 80%, at least 90%, at least 95% and most preferred essentially 100% as compared to the absence of the Tead1 antagonist.

Furthermore, a Tead1 antagonist may interact with one or more transcriptional regulators involved in controlling the expression of Tead1, e.g. one or more transcriptional regulators (e.g. a protein, such as a transcription factor), that interact with the Tead1 promoter or an enhancer (or the transcriptional complex bound to the Tead1 promoter or an enhancer) to selectively decrease the amount of Tead1 RNA or protein in skeletal muscle. It will be evident that a Tead1 antagonist may function by inhibiting (e.g. reducing or blocking) the activity or function of a transcriptional regulator that induces Ted1 expression. Alternatively, a Tead1 antagonist may function by increasing (e.g. promoting or enhancing) the function of a transcriptional regulator that reduces Tead1 expression.

In some embodiments, the Tead1 antagonist may be an agent that selectively reduces endogenous Tead1 expression by interacting with the Tead1 promoter or an enhancer (or the transcriptional complex bound to the Cytor promoter or an enhancer) directly. For instance, the Tead1 antagonist may comprise a polypeptide comprising a domain that binds to the Tead1 promoter that is operably linked to a domain comprising a transcriptional inhibitor.

In a representative example, the domain that binds to the Tead1 promoter or Tead1 gene may be a CRISPR associated protein (e.g. a CRISPR associated protein, e.g. Cas9 or Cpf1). Accordingly, the Tead1 antagonist (i.e. agent that decreases or prevents Tead1 expression in skeletal muscle) may further comprise a guide RNA capable of hydridising with the Tead1 promoter nucleic acid or the Tead1 gene.

The skilled person readily could design a suitable guide RNA based on the known sequence of the Tead1 promoter and gene as well as well-established guidance in the art for designing such molecules.

The skilled person readily would understand that the polypeptide defined above may be provided by administering one or more nucleic acid molecules encoding the polypeptide and optionally the guide RNA (and any other necessary nucleic acid molecule(s), e.g. a trans-enhancer nucleic acid), that is/are expressed in a cell (e.g. a myoblast cell), i.e. transcribed and/or translated in the cell.

In some embodiments the Tead1 antagonist or agent that selectively decreases or prevents Tead1 RNA and/or protein in skeletal muscle comprises a gene therapy, i.e. the administration of one or more nucleic acid molecules that function to selectively decrease the amount of Cytor RNA and/or protein in skeletal muscle.

It will be evident that numerous gene therapy strategies may be employed to decrease or prevent Tead1 RNA and/or protein in skeletal muscle using techniques that are known in the art. In some embodiments, the gene therapy may involve the administration of nucleic acid molecules that do not integrate into the genome of cells and result only in a transient change in the amount of Tead1 RNA and/or protein, e.g. a transient change in endogenous Tead1 expression.

As discussed above, it is possible to design Cytor agonists and/or Tead1 antagonists (e.g. nucleic acid molecules and/or polypeptides) based on known methods in the art as described above. However, the skilled person will understand that it is not necessary to understand the mechanism of action of a Cytor agonist or a Tead1 antagonist in order to work the invention. The skilled person readily could identify Cytor agonists and Tead1 antagonists suitable for use in the invention using routine experimentation based on techniques that are well-established in the art.

The Examples demonstrate that Cytor and Tead1 regulate myoblast differentiation into type II myotubes and show that an increase in Cytor RNA and/or a decrease in Tead1 RNA and/or protein in skeletal muscle results in an increase in both the size and number of type II myotubes. Accordingly, Cytor agonists and/or Tead1 antagonists may be identified and selected by screening agents that result in an increase in the amount of Cytor RNA and/or a decrease in the amount of Tead1 RNA or protein in myoblasts. Any suitable method may be used to screen agents that result in an increase in the amount of Cytor RNA and/or a decrease in the amount of Tead1 RNA or protein.

In a representative example, an in vitro method of identifying a Cytor agonist and/or a Tead1 antagonist may comprise:
(a) providing an agent or a plurality of agents that may function as a Cytor agonist and/or a Tead1 antagonist (e.g. a library of compounds, e.g. a drug screening library);
(b) contacting an agent from (a) with a myoblast cell (e.g. a plurality of myoblast cells);
(c) determining the amount of Cytor RNA and/or Tead1 RNA or protein in the myoblast cell(s);
(d) optionally repeating steps (b) and (c); and
(e) identifying an agent that results in an increase in Cytor RNA and/or a decrease in Tead1 RNA or protein in the myoblast cell(s) compared to control myoblast cells.

The method may further involve a step of verifying the activity of the agent by determining whether the agent is able to promote the differentiation of myoblasts into type II myotubes, e.g. to increase the size (e.g. area) and/or number of type II myotubes (e.g. relative to a suitable control). Any suitable method for verifying the activity of the agent may be used, e.g. based on the methods described in the Examples below.

In a further representative example an in vitro method of identifying a Cytor agonist and/or a Tead1 antagonist may comprise:
(a) providing an agent or a plurality of agents that may function as a Cytor agonist and/or a Tead1 antagonist (e.g. a library of compounds, e.g. a drug screening library);
(b) contacting an agent from (a) with a myoblast cell (e.g. a plurality of myoblast cells);
(c) measuring the size and/or number of type II myotubes after a suitable period of time (e.g. a period of time suitable for myoblast cells to differentiate into type II myotubes, e.g. following culture under conditions suitable for myoblast cells to differentiate into type II myotubes);
(d) optionally repeating steps (b) and (c); and
(e) identifying an agent that results in an increase in the size and/or number of type II myotubes compared to control myoblast cells.

The method may further involve a step of verifying the activity of the agent by determining whether the agent is able to increase the amount of Cytor RNA and/or to decrease the amount of Tead1 RNA or protein in myoblast cells relative to control myoblast cells. Any suitable method for verifying the activity of the agent may be used.

The term “agent” as used herein refers to a substance that induces a desired pharmacological and/or physiological effect. Suitably, an agent functions to increase, promote, enhance or potentiate the expression, activity or function of Cytor RNA in a myoblast, e.g. in skeletal muscle, e.g. functions to increase the amount of Cytor RNA in skeletal muscle. Also suitably, an agent functions to decrease, reduce, or prevent the expression, activity or function of Tead1 RNA and/or protein in a myoblast, e.g. in skeletal muscle, e.g. functions to decrease the amount of Tead1 RNA and/or protein in skeletal muscle. It follows that the term “agent” as used herein embraces Cytor agonists as well as Tead1 antagonists.

Alternatively viewed, an agent (a Cytor agonists or a Tead1 antagonist) functions to increase or promote the differentiation of myoblasts into type II myotubes, e.g. to increase or promote the size and/or number of type II myotubes, e.g. in a myoblast cell culture and/or in skeletal muscle. In a further aspect, the desired physiological effect is the promotion of myogenesis in the skeletal muscle of a subject, an increase in the skeletal muscle mass in a subject and/or an improvement in the skeletal muscle function in a subject. The term “agent” also encompasses pharmaceutically acceptable and pharmacologically active forms thereof, including salts.

An increase in the expression of Cytor RNA and/or a decrease in the expression of Tead1 RNA and/or protein in skeletal muscle or myoblast cells may be determined by any suitable means known in the art. In particular, the increase in Cytor RNA caused by the agent (i.e. Cytor agonist) and/or the decrease in the expression of RNA and/or protein is relative to a suitable control, e.g. skeletal muscle of a subject that has not been administered the agent or has been administered a control substance or myoblast cells that have not been contacted with the agent or have been contacted with a control substance. It will be evident to the skilled person that the effect of the agent on Cytor RNA and/or Tead1 RNA and/or protein in myoblast cells may be used as a proxy for the effect on skeletal muscle. Thus, an agent that increases the amount of Cytor RNA and/or decreases the amount of Tead1 RNA and/or protein in skeletal muscle also refers to an agent that increases the amount of Cytor RNA and/or decreases the amount of Tead1 RNA and/or protein in myoblast cells and these definitions may be used interchangeably herein.

Similarly, an increase in the the differentiation of myoblasts into type II myotubes, e.g. to increase or promote the size and/or number of type II myotubes, e.g. in a myoblast cell culture and/or in skeletal muscle may be determined by any suitable means known in the art. In particular, the increase in differentiation caused by the agent (i.e. Cytor agonist and/or Tead1 antagonist) is relative to a suitable control, e.g. skeletal muscle of a subject that has not been administered the agent or has been administered a control substance or myoblast cells that have not been contacted with the agent or have been contacted with a control substance.

The effects of the agent on myogenesis, skeletal muscle mass and skeletal muscle function may be measured using any suitable means known in the art. For instance, physical tests (e.g. walking tests, grip tests) may be used to measure the effects of the agent on muscle function. The effects on myogenesis and skeletal muscle mass may be determined using appropriate biomarkers (e.g. myosin gene expression) and/or by measuring muscle mass using imaging methods, e.g. MRI or CT scanning.

An agent (i.e. a Cytor agonist and/or Tead1 antagonist) may be a proteinaceous, non-proteinaceous (e.g. chemical entity) or nucleic acid molecule. Alternatively viewed, proteinaceous, non-proteinaceous (e.g. chemical entities) or nucleic acid molecules Cytor agonists and/or Tead1 antagonists may be used in treatments and methods described herein.

Proteinaceous molecules include peptides, polypeptides and proteins. The terms polypeptide and protein are used interchangeably herein.

Non-proteinaceous molecules include small, intermediate or large chemical molecules as well as molecules identified from natural product screening or the screening of chemical libraries. Thus, in some embodiments, the plurality of agents that may function as a Cytor agonist and/or Tead1 antagonist described above is a library (e.g. a drug screening library) of or comprising non-proteinaceous molecules compounds. Natural product screening includes the screening of extracts or samples from any suitable source of natural products including plants, microorganisms, soil, river beds, coral and aquatic environments for molecules or groups of molecules which have an effect on Cytor RNA activity and/or Tead1 activity, or the level of Cytor RNA (e.g. Cytor gene expression) and/or Tead1 RNA and/or protein (e.g. Tead1 gene expression). In some embodiments, the agent is a small or intermediate chemical molecule, e.g. a molecule with a molecular weight of about 1500 Da or less, e.g. about 1200 Da, 1100 Da, 1000 Da or less, such as about 100-1000 Da, 200-900 Da or 300-800 Da.

As shown in the Examples, Cytor RNA is an exercise-induced lncRNA. Whilst not wishing to be bound by theory, it is thought that agents (e.g. small or intermediate chemical molecules, e.g. drugs) that function as exercise mimetics, may result in a selective increase in Cytor RNA, i.e. may function as a Cytor agonist as defined herein. Exercise mimetics may be defined as pharmacological compounds or agents that are able to produce the benefits of fitness, such as mitochondrial remodeling effects including increased mitochondrial oxidative phosphorylation (OXPHOS) and fatty acid metabolism. For instance, muscle-targeted exercise mimetics offering exercise-mimicking benefits such as lower blood glucose levels, reduced inflammation and increased endurance have been developed and may also function as Cytor agonists. Accordingly, in some embodiments, the agent may be a drug that functions to increase the metabolic rate of cells (e.g. an exercise mimetics, such as a muscle targeted exercise mimetic), such as activators of AMP-activated protein kinase (AMPK), SIRT1 or PPARδ as well as ligands of REV-ERBα or ERRγ. Thus, in some embodiments, agents known to function to increase the metabolic rate of cells (e.g. an exercise mimetics, such as a muscle targeted exercise mimetic) may be used in the screening methods described above.

Nucleic acid molecule agents (“nucleic acids” or “polynucleotides”) include RNA, cDNA, genomic DNA, synthetic forms and mixed polymers, both sense and antisense strands (i.e. single and double stranded molecules), and may be chemically or biochemically modified or may contain non-natural or derivatized nucleotide bases, as will be readily appreciated by those skilled in the art. Also included are synthetic molecules that mimic polynucleotides in their ability to bind to a designated sequence via hydrogen binding and other chemical interactions. Such molecules are known in the art and include, for example, those in which peptide linkages substitute for phosphate linkages in the backbone of the molecule. In some embodiments, the nucleic acid molecules may be modified to increase their stability, e.g. relative to a corresponding unmodified nucleic acid. In particular, the nucleic acid molecule (e.g. RNA) may be modified to increase its resistance to degradation (e.g. by RNases). Suitable modifications are known in the art and any such modification may be used in the nucleic acid molecule agents of the invention.

In a preferred embodiment, the Cytor agonist is a nucleic acid molecule as defined above, e.g. Cytor RNA or a nucleic acid molecule (e.g. DNA molecule) encoding Cytor RNA. In a related preferred embodiment, the Tead1 agonist is a nucleic acid molecule or nucleotide-based inhibitor as defined above, e.g. a siRNA, shRNA or antisense molecule.

In the methods of identifying Cytor agonists and/or Tead1 agonists defined above, the step of contacting an agent from (a) with a myoblast cell (e.g. a plurality of myoblast cells) may comprise a step of contacting a cell culture comprising a plurality of myoblast cells under conditions suitable for the cells to express Cytor RNA and/or Tead1 RNA and protein. Suitable conditions may be determined as a matter of routine by culturing cells and determining whether they express Cytor RNA and/or Tead1 RNA and protein. The agent may be contacted in an amount that may be sufficient to affect the amount of Cytor RNA and/or Tead1 RNA and/or protein in the cells and will depend on the nature of the agent. The skilled person could determine a suitable amount of agent as a matter of routine experimentation.

The step of determining the amount of Cytor RNA and/or Tead1 RNA and/or protein in the myoblast cells may utilise any suitable methods known in the art. For instance, RNA (e.g. total RNA) and/or protein may be isolated from the cells and analysed to determine the amount of Cytor RNA and/or Tead1 RNA and/or protein present (e.g. by nucleic acid amplification and detection methods, e.g. real-time PCR, northern blotting etc.; or by protein detection methods, e.g. western blotting or mass spectrometry). Alternatively, the myoblast cells may be modified to incorporate a reporter molecule that is associated with Cytor RNA and/or Tead1 RNA and/or protein, such that the reporter molecule functions as a proxy for Cytor RNA and/or Tead1 RNA and/or protein and measuring the amount of the reporter molecule is indicative of the amount of Cytor RNA and/or Tead1 RNA and/or protein.

The step of measuring the size and/or number of type II myotubes after a suitable period of time may utilise any suitable methods known in the art. For instance, any cell microscopy methods may be used to count the number of type II myotubes and/or assess their size. It may not be necessary to measure the size and/or number of type II myotubes in the whole cell culture, i.e. a sample of the cell culture may be measured, e.g. the data may be used to extrapolate the number in the whole culture or may be compared to a corresponding sample from a control culture. It may be necessary or useful to stain the cells, e.g. myotubes, prior to measuring their size and/or number. Any suitable staining methods may be used.

Steps (b) and (c) of the methods above may be repeated for each agent in the plurality of agents, e.g. drug screening library, to identify multiple Cytor agonists and/or Tead1 antagonists. Thus, the method may be a high-throughput method and may utilise automated screening methods, e.g. robotics etc. Similarly, the method may be repeated (e.g. 2, 3, 4 or more times) for each agent to improve the accuracy of the method, i.e. to eliminate false positives or agents that have only a small effect on Cytor expression and/or Tead1 expression.

Identifying an agent that results in an increase in Cytor RNA, decrease in Tead1 RNA and/or protein, or in the size and/or number of type II myotubes compared to control myoblast cells involves comparing the effect of the agent on the myoblast cells to suitable control cells. The skilled person readily can determine which cells may be used as a control. For example, suitable control cells may be untreated cells or cells treated with an agent that does not result in an increase in Cytor RNA or in the size, decrease in Tead1 RNA and/or protein, and/or number of type II myotubes, e.g. the solvent (e.g. water or buffer) in which the agent is dissolved.

Functionally-equivalent variant Cytor RNA includes natural biological variants (e.g. allelic variants or splice variants within a species) as well as polynucleotides that are related to, or derived from, a naturally-occurring nucleic acid molecule. Functionally-equivalent Cytor RNA may be obtained by modifying a native polynucleotide sequence by single or multiple (e.g. 2-50, preferably 2-30 or 2-20) nucleotide mutations (i.e. substitutions, additions and/or deletions), but without destroying the molecule's function and/or overall structure. As a representative example, a functionally-equivalent variant polynucleotide (e.g. RNA) may contain one or more nucleotide mutations that do not eliminate the myogenic activity of the molecule, i.e. the capacity to promote the differentiation of myoblasts into type II myotubes. In some embodiments, the functionally-equivalent variant has at least 50%, preferably at least 70%, 80% or 90% of the myogenic activity of the related RNA. The activity of variant polynucleotides can be determined using methods that are routine in the art, e.g. expressing the variant RNA in a myoblast cell and determining the effect on differentiation into type II myotubes (e.g. the size and/or number of type II myotubes) relative to a myoblast cell expressing wild-type Cytor RNA under the same conditions.

Similarly, a functionally-equivalent portion or fragment refers to a polynucleotide (e.g. RNA) comprising a portion or fragment of a Cytor nucleotide sequence as defined herein, which may include a functionally-equivalent variant. A “portion” or “fragment” comprises at least 600, 650, 700, 750, 800 or more nucleotides of the sequence from which it is derived. Alternatively viewed, a portion or fragment retains at least about 70%, e.g. at least about 75%, 80%, 85%, 90% or 95% of the sequence from which it is derived. Said portion may be obtained from a central or 5′ or 3′ portion of the sequence. As a representative example, a functionally-equivalent portion (e.g. RNA) retains the myogenic activity of the equivalent full-length molecule, i.e. the capacity to promote the differentiation of myoblasts into type II myotubes. In some embodiments, the functionally-equivalent portion has at least 50%, preferably at least 70%, 80% or 90% of the myogenic activity of the related RNA (i.e. the RNA from which it is derived). The activity of polynucleotide portions or fragments can be determined using methods that are routine in the art, e.g. expressing the RNA portion in myoblast cells and determining the effect on differentiation into type II myotubes (e.g. the size and/or number of type II myotubes) relative to myoblast cells expressing wild-type (i.e. full-length) Cytor RNA under the same conditions.

The term “operably linked” refers to a functional linkage between two or more elements, e.g. polynucleotide or polypeptide elements. For example, an operable linkage between a polynucleotide encoding a Cytor RNA and a regulatory sequence (i.e. a promoter) is functional link that allows for the expression of the polynucleotide. Operably linked elements may be contiguous or non-contiguous.

The nucleic acid molecule agents of the invention may be Cytor RNA (or a functionally-equivalent fragment and/or variant thereof) or may be a nucleic acid molecule that encodes Cytor RNA or an alternative Cytor agonist, e.g. a polypeptide agonist such as a transcriptional activator. Thus, in some embodiments, the nucleic acid molecule agents of the invention may be administered as a suitable genetic construct as described below and delivered to the subject where it is expressed, e.g. to provide Cytor RNA directly. Typically, the nucleic acid in the genetic construct is operably linked to a promoter such that the nucleic acid is expressed in the cell. The genetic constructs of the invention can be prepared using methods well known in the art, for example in Sambrook et al (2001).

Genetic constructs for delivery of polynucleotides can be DNA or RNA. In some embodiments, they are DNA.

Preferably, the genetic construct is adapted for delivery to an animal (e.g. human) cell, particularly a myoblast cell. For instance, transposon systems (e.g. sleeping beauty, piggyBac etc.) may be used to integrate the nucleic acid molecule agent into the cell (e.g. myoblast cell) genome. Means and methods of introducing a genetic construct into a cell are known in the art and include the use of nanoparticles, such as immunoliposomes, liposomes, viral vectors (including vaccinia, modified vaccinia, lentivirus, parvovirus, retroviruses (e.g. replication defective retroviruses), adenovirus and adeno-associated viral (AAV) vectors). The term “viral vector” refers to a virus particle containing a nucleic acid molecule “vector”.

Furthermore, methods of delivering polynucleotides to a target tissue of a subject (patient) for treatment are also well known in the art. For instance, a high-efficiency nucleic acid delivery system that uses receptor-mediated endocytosis to carry DNA macromolecules into cells may be employed. This may be accomplished by conjugating the iron-transport protein transferrin to polycations that bind nucleic acids. High-efficiency receptor-mediated delivery of the DNA constructs or other genetic constructs of the invention using the endosome-disruption activity of defective or chemically inactivated adenovirus particles produced by the methods of Cotten et al (1992) Proc. Natl. Acad. Sci. USA 89, 6094-6098 may also be used. It will be appreciated that “naked DNA” and DNA complexed with cationic and neutral lipids may also be useful in introducing the DNA of the invention into cells of the individual to be treated. Non-viral approaches to gene therapy are described in Ledley (1995, Human Gene Therapy 6, 1129-1144).

Although it may be useful to use tissue-specific promoters in the vectors encoding or comprising the nucleic acid molecule agents of the invention, this is not essential, as the risk of expression of the nucleic acid molecule agent in the body at locations other than the desired site of action, e.g. skeletal muscle, would be expected to be tolerable in compared to the therapeutic benefit to a patient suffering from a disease or condition associated with muscle atrophy. It may be desirable to be able to temporally regulate expression of the nucleic acid molecule agent in the cell, although this is also not essential, and can be achieved using any suitable means known in the art.

Thus, in some embodiments, the Cytor agonist or agent of the invention is a vector comprising the nucleic acid molecule as described above. In some embodiments, the Cytor agonist or agent of the invention is a viral vector (e.g. a virus) comprising the nucleic acid molecule as described herein.

The term “myogenesis” refers to the formation of muscle or muscular tissue. Muscle fibres form through the fusion of myoblasts into multinucleated fibres termed “myotubes”. The terms “muscle fibre” and “myotube” are used interchangeably herein.

There are two main types of myotubes (muscle fibres) termed “type I” (“slow”) and “type II” (“fast” or “fast twitch”) myotubes. As shown in the Examples, increasing the amount of Cytor RNA and/or decreasing the amount of Tead1 RNA and/or protein in skeletal muscle (e.g. in myoblasts) results in an increase in the size (e.g. area and/or diameter) and number of type II myotubes, e.g. Cytor RNA functions to promote the differentiation of myoblasts into type II myotubes.

Thus, in some embodiments, the Cytor agonist and/or Tead1 antagonist of the invention may be used to promote myogenesis in skeletal muscle of a subject. In particular, the Cytor agonist and/or Tead1 antagonist of the invention may be used to promote type II myogenesis in skeletal muscle of a subject, i.e. to increase the size and/or number of type II myotubes (muscle fibres, i.e. so-called “fast-twitch” muscle fibres) in skeletal muscle. As noted above, an increase in the size and/or number of type II myotubes may be determined using any suitable means known in the art. In particular, the increase may be determined by comparing the size and/or number of type II myotubes in the treated subject to a suitable control, e.g. the size and/or number of type II myotubes in the skeletal muscle of the subject prior to treatment. The increase may be a relative increase, e.g. an increase in the proportion of type II myotubes in the skeletal muscle, or may be an absolute increase, e.g. an increase in the overall number and/or size of type II myotubes. In some preferred embodiments, the increase is an absolute increase.

In some embodiments, the Cytor agonist and/or Tead1 antagonist of the invention results in at least about a 1%, 2%, 3%, 4% or 5% increase in the number and/or size of type II myotubes in the skeletal muscle of the subject, such as about a 6%, 7%, 8%, 9%, 10% or more increase in the number and/or size of type II myotubes, e.g. about 12%, 15%, 20% or more.

An increase in the size and/or number of type II myotubes may result in an increase in the skeletal muscle mass of the subject, although the total mass of the subject may not increase (e.g. due to the increased metabolic demand associated with an increase in muscle mass). Thus, in some embodiments, the Cytor agonist and/or Tead1 antagonist of the invention may be used to increase skeletal muscle mass in a subject. An increase in skeletal muscle mass may be determined using any suitable means known in the art, e.g. muscle imaging techniques, such as MRI or CT scans. In particular, the increase may be determined by comparing the skeletal muscle mass in the treated subject to a suitable control, e.g. the mass the skeletal muscle of the subject prior to treatment.

In some embodiments, the Cytor agonist and/or Tead1 antagonist of the invention results in at least about a 1%, 2%, 3%, 4% or 5% increase muscle mass of the skeletal muscle of the subject, such as about a 6%, 7%, 8%, 9%, 10% or more increase muscle mass, e.g. about 12%, 15%, 20% or more.

It will be evident that an increase in the size and/or number of type II myotubes and/or an increase in skeletal muscle mass may result in an improvement in the function of the skeletal muscle of a subject. Thus, in some embodiments, the Cytor agonist and/or Tead1 antagonist of the invention may be used to improve skeletal muscle function in a subject. An improvement in skeletal muscle function may be an increase in the strength of the muscle. An increase in skeletal muscle function, e.g. strength, may be determined using any suitable means known in the art, e.g. physical tests, such as walking tests or grip strength tests. In particular, the increase may be determined by comparing the skeletal muscle function, e.g. strength, in the treated subject to a suitable control, e.g. the function (e.g. strength) of the skeletal muscle of the subject prior to treatment.

In some embodiments, the Cytor agonist and/or Tead1 antagonist of the invention results in at least about a 1%, 2%, 3%, 4% or 5% increase skeletal muscle function of the subject, such as about a 6%, 7%, 8%, 9%, 10% or more increase muscle mass, e.g. about 12%, 15%, 20% or more.

The increases described above may be an increase in a discrete area of skeletal muscle, e.g. a specific muscle or muscle group, or may be an increase in the overall skeletal muscle of the subject.

It will be evident that the Cytor agonist and/or Tead1 antagonist of the invention may have a combination of effects on skeletal muscle and these effects may be linked. For instance, promoting myogenesis may be expected to result in an increase in muscle mass and/or function. Thus, in some embodiments, the Cytor agonist and/or Tead1 antagonist may be for use in promoting myogenesis and muscle mass and/or muscle function. In some embodiments, the Cytor agonist and/or Tead1 antagonist may be for use in increasing skeletal muscle mass and function.

There are numerous diseases and conditions known in the art in which subjects suffer from a loss of type II muscle fibres, i.e. a reduction in the number and/or size of type II muscle fibres (myotubes). A loss of muscle fibres (including type II muscle fibres), resulting in a loss of muscle mass and/or function (e.g. strength) may also be termed “muscle atrophy”. Accordingly, the Cytor agonists and/or Tead1 antagonist of the invention are expected to find utility in the treatment and prevention of diseases and conditions associated with a loss of muscle fibres, particularly type II muscle fibres, or muscle atrophy and/or muscle dysfunction.

The term “muscle dysfunction” refers to skeletal muscles that show reduced strength and/or reduced endurance, i.e. muscles that are unable to perform their physiological tasks adequately. Muscle dysfunction can be expressed as fatigue and/or weakness. Thus, in some embodiments the Cytor agonists and/or Tead1 antagonists of the invention are expected to find utility in the treatment and/or prevention of muscle dysfunction, e.g. treating or preventing muscle fatigue and/or weakness. Alternatively viewed, the Cytor agonists and/or Tead1 antagonists of the invention may find utility in increasing muscle strength and/or endurance.

Thus, in some embodiments, the invention provides a Cytor agonist and/or Tead1 antagonist as defined herein for use in treating or preventing a loss of muscle fibres (e.g. type II muscle fibres or myotubes) in a subject. Alternatively viewed, the invention provides a Cytor agonist and/or Tead1 antagonist as defined herein for use in treating or preventing skeletal muscle atrophy in a subject. In a further embodiment, the invention provides a Cytor agonist and/or Tead1 antagonist as defined herein for use in treating or preventing a disease associated with a loss of muscle fibres (e.g. type II muscle fibres or myotubes) or treating or preventing a disease associated with muscle atrophy, i.e. where a symptom of the disease or condition is a loss of muscle fibres (e.g. type II muscle fibres or myotubes) or muscle atrophy. Methods of treating or preventing these conditions using a Cytor agonist and/or Tead1 antagonist as defined herein also form aspects of the invention.

A loss of muscle fibres or muscle atrophy may be associated with a lack or absence of physical activity. A loss of muscle fibres or muscle atrophy is also associated with ageing, which may be compounded by a reduction in physical activity. Thus, some subjects may have characteristics associated with an increased risk of developing skeletal muscle atrophy.

For instance, the age, activity, genetic characteristics (e.g. genotype), ethnicity and/or sex of the subject may be indicative of an increased risk of developing skeletal muscle atrophy, particularly in combination with an additional underlying condition as described below, e.g. a condition that renders the subject inactive or immobile.

Thus, in some embodiments, the subject to be treated has skeletal muscle atrophy (e.g. which is at risk of worsening) or is at risk (e.g. increased risk) of developing skeletal muscle atrophy, i.e. a loss of muscle fibres (e.g. type II muscle fibres).

In some embodiments, the subject with skeletal muscle atrophy (e.g. which is at risk of worsening) or at risk (e.g. increased risk) of developing skeletal muscle atrophy is at least 45 years old, e.g. 50, 55, 60, 65 years old or above (i.e. an aged or ageing, e.g. an elderly, subject). Alternatively viewed, the subject to be treated may be at least 45 years old, e.g. 50, 55, 60, 65 years old or above (i.e. an aged or ageing e.g. an elderly, subject). In some particular embodiments, the subject selected for a preventative treatment, i.e. to prevent the development of muscle atrophy or worsening (a deterioration of) muscle atrophy is at least 45 years old, e.g. 50, 55, 60, 65 years old or above.

In some embodiments, the subject with skeletal muscle atrophy (e.g. which is at risk of worsening) or at risk (e.g. increased risk) of developing skeletal muscle atrophy is inactive or immobile. Alternatively viewed, the subject to be treated may be inactive or immobile. A subject may be inactive or immobile due to an underlying condition or injury that results in a loss of mobility. For instance, the subject may be obese (e.g. morbidly obese) or physically disabled, e.g. they may have a brain or spinal cord injury, multiple sclerosis, cerebral palsy, a respiratory disorder, epilepsy or a hearing or visual impairment that results in a loss of mobility.

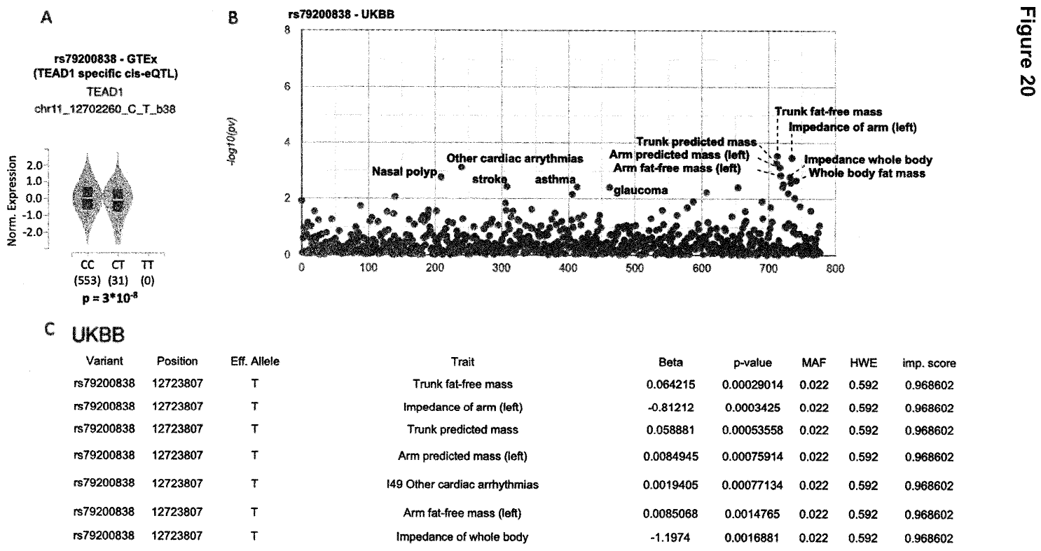

As shown in the Examples and FIG. 20, the inventors have identified a genetic marker that is associated with an increased risk of muscle atrophy, particularly in aging subject, e.g. associated with an increased risk of developing sarcopenia. The genetic marker is the cis-eQTL rs74360723, wherein subjects that are heterozygous or homozygous for the G allele of cis-eQTL rs74360724 have an increased risk of developing muscle atrophy, e.g. sarcopenia, relative to subjects that are homozygous for the A allele of cis-eQTL rs74360724. Alternatively or in addition, the genetic marker is the cis-eQTL rs 79200838, wherein subjects that are heterozygous or homozygous for the A allele of cis-eQTL rs79200838 have an decreased risk of developing muscle atrophy, e.g. sarcopenia, relative to subjects that are homozygous for the C allele of cis-eQTL rs79200838. Thus, in some embodiments the subject with skeletal muscle atrophy (e.g. which is at risk of worsening) or at risk (e.g. increased risk) of developing skeletal muscle atrophy is heterozygous or homozygous (particularly homozygous) for the G allele of cis-eQTL rs74360724. Alternatively viewed, the subject to be treated may be heterozygous or homozygous (particularly homozygous) for the G allele of cis-eQTL rs74360724. Similarly, in some embodiments the subject with skeletal muscle atrophy (e.g. which is at risk of worsening) or at risk (e.g. increased risk) of developing skeletal muscle atrophy is heterozygous or homozygous (particularly homozygous) for the Callele of cis-eQTL rs79200838. Alternatively viewed, the subject to be treated may be heterozygous or homozygous (particularly homozygous) for the C allele of cis-eQTL rs79200838.

In some embodiments, a subject may be selected for preventative treatment with a Cytor agonist and/or a Tead1 antagonist of the invention based on their genotype, i.e. their cis-eQTL rs74360724 and/or cis-eQTL rs79200838 genotype. Thus, in a further aspect the invention provides a method for selecting or identifying a subject for treatment according to the invention comprising determining the genotype of the subject at the cis-eQTL rs74360724, wherein when the subject is heterozygous or homozygous for the G allele or at the cis-eQTL rs79200838, wherein when the subject is heterozygous or homozygous for the C allele the subject is selected for treatment with a Cytor agonist and/or a Tead1 antagonist.

Thus, in some embodiments, the method may be viewed as determining the suitability of a subject for treatment according to the invention.

Thus, in some preferred embodiments, the method is for selecting a subject (determining the suitability of a subject) for preventative treatment, i.e. to prevent the development of skeletal muscle atrophy or a deterioration of existing skeletal muscle atrophy (e.g. sarcopenia).

A disease or condition associated with a loss of muscle fibres (e.g. type II muscle fibres or myotubes) or muscle atrophy refers to any condition in which a loss of muscle fibres (particularly type II muscle fibres) is observed. Suitably, a disease or condition associated with a loss of muscle fibres (e.g. type II muscle fibres or myotubes) or muscle atrophy includes sarcopenia, cachexia (e.g. cancer cachexia or AIDS cachexia), sepsis, diabetes (particularly type I diabetes), muscular dystrophy (e.g. Duchenne Muscular Dystrophy, Becker's MD, Congenital MD, Myotonic dystrophy, Limb-girdle MD, Emery-Dreifuss MD, Distal MD and Facioscapulohumeral Muscular Dystrophy), Chronic Heart Failure (e.g. diaphragm muscle atrophy resulting from Chronic Heart Failure), Chronic Obstructive Pulmonary Disease (e.g. diaphragm muscle atrophy resulting from Chronic Obstructive Pulmonary Disease), Amyotrophic Lateral Sclerosis (ALS), Spinal Muscular Atrophy, Guillain-Barré syndrome, intensive care unit-acquired weakness (ICUAW), immobilization resulting from bone fractures (e.g. hip (femur neck) fractures) and immobilization resulting from an infection (e.g. a bacterial or viral infection, such as a coronavirus infection, e.g. COVID-19).

Thus, in some embodiments, the invention provides a Cytor agonist and/or Tead1 antagonist as defined herein for use in treating or preventing a loss of muscle fibres (e.g. type II muscle fibres or myotubes) or muscle atrophy in a subject with (i.e. suffering from) or is at risk of developing sarcopenia, cachexia (e.g. cancer cachexia or AIDS cachexia), sepsis, diabetes (particularly type I diabetes), muscular dystrophy (e.g. Duchenne Muscular Dystrophy, Becker's MD, Congenital MD, Myotonic dystrophy, Limb-girdle MD, Emery-Dreifuss MD, Distal MD and Facioscapulohumeral Muscular Dystrophy), Chronic Heart Failure (e.g. diaphragm muscle atrophy resulting from Chronic Heart Failure), Chronic Obstructive Pulmonary Disease (e.g. diaphragm muscle atrophy resulting from Chronic Obstructive Pulmonary Disease), Amyotrophic Lateral Sclerosis (ALS), Spinal Muscular Atrophy, Guillain-Barré syndrome, intensive care unit acquired weakness, immobilization resulting from bone fractures (e.g. hip (femur neck) fractures) or immobilization resulting from an infection.

In a further embodiment, the invention provides a Cytor agonist and/or Tead1 antagonist as defined herein for use in treating or preventing starvation, sarcopenia, cachexia (e.g. cancer cachexia or AIDS cachexia), sepsis, diabetes (particularly type I diabetes), muscular dystrophy (e.g. Duchenne Muscular Dystrophy, Becker's MD, Congenital MD, Myotonic dystrophy, Limb-girdle MD, Emery-Dreifuss MD, Distal MD and Facioscapulohumeral Muscular Dystrophy), Chronic Heart Failure (e.g. diaphragm muscle atrophy resulting from Chronic Heart Failure), Chronic Obstructive Pulmonary Disease (e.g. diaphragm muscle atrophy resulting from Chronic Obstructive Pulmonary Disease), Amyotrophic Lateral Sclerosis (ALS), Spinal Muscular Atrophy, Guillain-Barré syndrome, intensive care unit acquired weakness, immobilization resulting from bone fractures (e.g. hip (femur neck) fractures) or immobilization resulting from an infection.

Among the above-discussed diseases Duchenne Muscular Dystrophy, Myotonic dystrophy, type 2, Facioscapulohumeral Muscular Dystrophy, sepsis, cachexia, starvation are preferred since they are associated with fast-twitch muscle fibers. As discussed above, a loss of fast-twitch muscle fibers may particularly well be treated by a Tead1 antagonist.

Methods of treating or preventing these conditions using a Cytor agonist and/or Tead1 antagonist as defined herein also form aspects of the invention.

Sarcopenia refers to a syndrome characterized by progressive and generalized loss of skeletal muscle mass and strength. Sarcopenia may by defined as the presence of low skeletal muscle mass and either low muscle strength (e.g., handgrip) or low muscle performance (e.g., walking speed or muscle power); when all three conditions are present, severe sarcopenia may be diagnosed. Thus, in some embodiments, the Cytor agonist and/or a Tead1 antagonist is for use in treating or preventing severe sarcopenia.

In a representative embodiment, the diagnosis of sarcopenia can be assessed by measuring muscle mass, e.g. using dual energy X-Ray absorptiometry. The subject may be diagnosed as having sarcopenia if their muscle mass is considered to be low, e.g. where the percentage of muscle mass divided by height squared is below two standard deviations of the normal young mean (<7.23 kg/m² in men and in women at <5.67 kg/m²) as defined using dual energy X-Ray absorptiometry. Walking speed and/or grip strength may be used as primary indicators for sarcopenia, which is confirmed by measuring muscle mass. For example a diagnosis of sarcopenia (e.g. in an aged, i.e. elderly, subject) can be carried out by assessing the following parameters:
1. Measure walking speed. If walking speed for the subject (e.g. elderly, such as >65 years) is below 0.8 m/s at the 4-m walking test, measure the muscle mass.
2. If the walking speed at the 4-m walking test is higher than 0.8 m/s the hand-grip strength should be tested; if this value is lower than 20 Kg in women and 30 Kg in man, the muscle mass must be analyzed as described above.

Cachexia, also known as “wasting syndrome” is a complex syndrome associated with an underlying illness that causes ongoing muscle loss that is not entirely reversed with nutritional supplementation. Numerous diseases can cause cachexia, including cancer, congestive heart failure (CHF), chronic obstructive pulmonary disease (COPD), chronic kidney disease and AIDS. It is thought that systemic inflammation from these diseases may cause detrimental changes to metabolism and therefore body composition, including muscle mass. Thus, in some embodiments, cachexia is cancer cachexia, CHF cachexia, COPD cachexia, chronic kidney disease cachexia or AIDS cachexia.

Intensive care unit-acquired weakness (ICUAW) refers to weakness that is classified into three component conditions: critical illness polyneuropathy (CIP), critical illness myopathy (CIM), and critical illness neuromyopathy (CINM). CIP and CIM frequently co-exist (CINM) and when present separately cannot be reliably distinguished clinically. ICUAW may result from numerous conditions that result in a prolonged period under intensive care, particularly involving prolonged mechanical ventilation. In particular, ICUAW is prominent in subjects with severe sepsis or multiple organ failure.

Muscular dystrophy (MD) refers to a group of muscle diseases that results in increasing weakening and breakdown of skeletal muscles over time. MD includes Duchenne Muscular Dystrophy, Becker's MD, Congenital MD, Myotonic dystrophy, Limb-girdle MD, Emery-Dreifuss MD, Distal MD and Facioscapulohumeral Muscular Dystrophy.

Spinal muscular atrophy (SMA) refers to a heterogeneous group of rare debilitating disorders characterised by the degeneration of lower motor neurons (neuronal cells situated in the anterior horn of the spinal cord) and subsequent atrophy (wasting) of various muscle groups in the body. SMA includes proximal spinal muscular atrophies and distal spinal muscular atrophies. In some embodiments, SMA is autosomal recessive proximal spinal muscular atrophy.

Guillain-Barré syndrome is an acute (rapid-onset) muscle weakness caused by the immune system damaging the peripheral nervous system which typically results in muscle weakness, beginning in the feet and hands, which subsequently spreads to the arms and upper body.

Amyotrophic lateral sclerosis (ALS) (also known as motor neuron disease or Lou Gehrig's disease) refers to a debilitating disease with varied etiology characterized by rapidly progressive weakness, muscle atrophy and fasciculations, muscle spasticity, difficulty speaking (dysarthria), difficulty swallowing (dysphagia), and difficulty breathing (dyspnea).

The terms “treating” or “treatment” as used herein refer broadly to any effect or step (or intervention) beneficial in the management of a clinical condition or disorder. Treatment therefore may refer to reducing, alleviating, ameliorating, slowing the development of, or eliminating one or more symptoms of the disease, condition or syndrome that is being treated, relative to the symptoms prior to treatment, or in any way improving the clinical status of the subject. A treatment may include any clinical step or intervention which contributes to, or is a part of, a treatment programme or regimen.

A treatment may include delaying, limiting, reducing or preventing the onset of one or more symptoms of the disease, condition or syndrome (i.e. muscle fibre loss, muscle atrophy), for example relative to the symptom prior to the treatment. Thus, treatment explicitly includes both absolute prevention of occurrence or development of symptoms of the disease, condition or syndrome, and any delay in the development of the disease, condition or syndrome or symptom thereof, or reduction or limitation on the development or progression of the disease, condition or syndrome or symptom thereof.

As discussed further in the Examples, it is thought that increasing the amount of Cytor RNA and/or decreasing the amount of Tead1 RNA and/or protein in skeletal muscle (and/or its activity) functions to promote the differentiation of myoblasts into type II myotubes, thereby increasing the number and/or size of type II myotubes and/or muscle mass (e.g. type II muscle fibre mass) and rejuvenating the muscle tissue. Thus, the term “treatment” does not necessarily imply cure of the disease or condition or complete rejuvenation of the muscle tissue or symptoms thereof.

The terms “subject” and “patient” are used interchangeably herein and refer to a mammal, preferably a human. In some embodiments (e.g. where the agonist is used to increase muscle mass in a subject), the subject is a non-human animal, particularly a domesticated or farmed animal, e.g. livestock (such as poultry, pigs, cattle, sheep or goats) or farmed fish (e.g. salmon, tilapia or tuna).

The Cytor agonist and/or Tead1 antagonist may be provided in pharmaceutical composition, which may be formulated according to any of the conventional methods known in the art and widely described in the literature. Thus, a Cytor agonist and/or Tead1 antagonist may be incorporated, optionally together with other active (e.g. therapeutic) substances, with one or more conventional carriers, diluents and/or excipients. A pharmaceutical composition comprising a Cytor agonist and/or Tead1 antagonist as defined herein represents a further aspect of the invention.

The pharmaceutical composition described herein may be administered systemically or locally to the subject using any suitable means and the route of administration will depend on formulation of the pharmaceutical composition.

In some embodiments, systemic administration may be particularly useful. “Systemic administration” includes any form administration in which the agent (i.e. Cytor agonist and/or Tead1 antagonist) is administered to the body resulting in the whole body receiving the administered agent. Conveniently, systemic administration may be via enteral delivery (e.g. oral) or parenteral delivery (e.g. intravenous, intramuscular, subcutaneous, intratracheal, endotracheal, inhalation).

“Local administration” refers to administration of the Cytor agonist and/or Tead1 antagonist at the primary site of muscle loss (e.g. one or more skeletal muscles or muscle groups) or in the local vicinity of the primary site of muscle loss, e.g. via intramuscular injection. However, it will be evident that some forms of local administration may result in the whole body receiving the administered agent. Thus, in some embodiments, the Cytor agonist and/or Tead1 antagonist may be administered to provide an initial local effect and subsequent systemic effect.

Reference to “systemic administration” includes intra-articular, intravenous, intramuscular, intraperitoneal, and subcutaneous injection, infusion, as well as administration via oral, rectal and nasal routes, or via inhalation. Administration by intramuscular subcutaneous injection or via intravenous injection or infusion is particularly preferred.

Thus, in some embodiments, the Cytor agonist and/or Tead1 antagonist may be provided and/or formulated for intravenous, subcutaneous, transdermal or intramuscular administration. In some embodiments, the Cytor agonist and/or Tead1 antagonist (i.e. pharmaceutical composition) is administered by intramuscular administration.

The pharmaceutical composition may include one or more excipient, diluent, binder, lubricant, glidant, disintegrant, desensitizing agent, emulsifier, solubilizer, suspension agent, viscosity modifier, ionic tonicity agent, buffer, carrier, surfactant, flavor, or mixture thereof.

Examples of suitable pharmaceutical carriers, excipients and/or diluents are well-known in the art and include, but are not limited to, a gum, a starch (e.g. corn starch, pregeletanized starch), a sugar (e.g., lactose, mannitol, sucrose, dextrose), a cellulosic material (e.g. microcrystalline cellulose), an acrylate (e.g. polymethylacrylate), calcium carbonate, magnesium oxide, or mixtures thereof.

Pharmaceutically acceptable carriers for liquid formulations are aqueous or non-aqueous solutions, suspensions, emulsions or oils. Examples of non-aqueous solvents are propylene glycol, polyethylene glycol, and injectable organic esters such as ethyl oleate. Examples of oils are those of animal, vegetable, or synthetic origin, for example, peanut oil, soybean oil, olive oil, sunflower oil, fish-liver oil, another marine oil, or a lipid from milk or eggs.

Aqueous carriers include water, alcoholic/aqueous solutions, emulsions or suspensions, including saline and buffered media such as phosphate buffered saline solutions, water, emulsions, such as oil/water emulsions, various types of wetting agents, sterile solutions etc. Compositions comprising such carriers can be formulated by well-known conventional methods. Suitable carriers may comprise any material which, when combined with the Cytor agonist and/or Tead1 antagonist, retains the biological activity.

Preservatives and other additives may also be present including, for example, antimicrobials, anti-oxidants, chelating agents, and inert gases and the like. In addition, the pharmaceutical composition may comprise proteinaceous carriers like, e.g., serum albumin or immunoglobulin, preferably of human origin.

In some embodiments, the pharmaceutical composition may also be administered as a controlled-release composition, i.e. a composition in which the active ingredient is released over a period of time after administration. Controlled- or sustained-release compositions include formulation in lipophilic depots (e.g. fatty acids, waxes, oils). In another embodiment, the composition is an immediate-release composition, i.e. a composition in which all the active ingredient is released immediately after administration. Further examples for suitable formulations are provided in WO 2006/085983, the entire contents of which are incorporated by reference herein. For example, the pharmaceutical composition may be provided as liposomal formulations. The technology for forming liposomal suspensions is well-known in the art. The lipid layer employed can be of any conventional composition and can either contain cholesterol or can be cholesterol-free. The liposomes can be reduced in size, as through the use of standard sonication and homogenization techniques. Liposomal formulations containing the agent or pharmaceutical composition can be lyophilized to produce a lyophilizate which can be reconstituted with a pharmaceutically acceptable carrier, such as water, to regenerate a liposomal suspension.

In some embodiments, the pharmaceutical composition is a “ready to use” formulation that contains the Cytor agonist and/or Tead1 antagonist in dissolved or solubilized form and is intended to be used as such or upon further dilution in pharmaceutically acceptable (e.g. intravenous) diluents. However, in some embodiments, the pharmaceutical composition may be provided in a solid form, e.g. as a lyophilizate, to be dissolved in a suitable solvent to provide a liquid formulation.

In some embodiments, the Cytor agonist and/or Tead1 antagonist (e.g. small molecule) may be in the form of a salt, i.e. a pharmaceutically acceptable salt. For instance, the Cytor agonist and/or Tead1 antagonist may be in the form of an acidic or basic salt. In some embodiments, the Cytor agonist and/or Tead1 antagonist is in a neutral salt form.

Pharmaceutically acceptable salts include pharmaceutical acceptable base addition salts and acid addition salts, for example, metal salts, such as alkali and alkaline earth metal salts, ammonium salts, organic amine addition salts, and amino acid addition salts, and sulfonate salts. Acid addition salts include inorganic acid addition salts such as hydrochloride, sulfate and phosphate, and organic acid addition salts such as alkyl sulfonate, arylsulfonate, acetate, maleate, fumarate, tartrate, citrate and lactate. Examples of metal salts are alkali metal salts, such as lithium salt, sodium salt and potassium salt, alkaline earth metal salts such as magnesium salt and calcium salt, aluminum salt, and zinc salt. Examples of ammonium salts are ammonium salt and tetramethylammonium salt. Examples of organic amine addition salts are salts with morpholine and piperidine. Examples of amino acid addition salts are salts with glycine, phenylalanine, glutamic acid and lysine. Sulfonate salts include mesylate, tosylate and benzene sulfonic acid salts.

“Pharmaceutically acceptable” as referred to herein refers to ingredients that are compatible with other ingredients used in the methods or uses of the invention as well as physiologically acceptable to the recipient.

The Cytor agonist and/or Tead1 antagonist as defined herein may be administered to the subject in an effective amount, i.e. a therapeutically effective amount. The terms “effective amount” and “therapeutically effective amount” as used herein mean a sufficient amount of an Cytor agonist and/or Tead1 antagonist that provides the desired therapeutic or physiological effect or outcome by increasing the amount and/or activity of Cytor RNA and/or by decreasing the amount and/or activity of Tead1 RNA and/or protein (directly or indirectly as defined above). In addition, the effect may be an amelioration of the symptoms of the disease or condition (e.g. muscle atrophy) described here and/or complications arising from same or manifestations thereof. Undesirable effects, e.g. side effects, may sometimes manifest along with the desired therapeutic effect; hence, a practitioner balances the potential benefits against the potential risks in determining what is an appropriate “effective amount”.

The exact amount of Cytor agonist and/or Tead1 antagonist required will vary from subject to subject, depending on the numerous parameters such as age and general condition of the subject, mode of administration, weight, body surface area, sex, other drugs being administered concurrently and the like. Thus, it may not be possible to specify an exact “effective amount”. However, an appropriate “effective amount” in any individual case may be determined by one of ordinary skill in the art using routine experimentation.

The dosage regimen of the Cytor agonist and/or Tead1 antagonist will be determined by the attending physician and clinical factors. As is well known in the medical arts, dosages for any one subject depend upon many factors, including the subject's size, body surface area, age, the particular compound to be administered, sex, time and route of administration, general health, and other drugs being administered concurrently.

By way of example, in some embodiments of the invention the Cytor agonist and/or Tead1 antagonist is administered daily or every 2-3 days. In some embodiments, the Cytor agonist or agent may be administered for at least three days, e.g. for 3, 4, 5, 6, 7, 8, 9 10 or more days (e.g. 20, 25, 30 or more days). This administration may be in a single cycle or in total in multiple cycles.

In some embodiments of the invention the Cytor agonist and/or Tead1 antagonist is administered weekly or every 2-3 weeks. In some embodiments, the Cytor agonist and/or Tead1 antagonist may be administered for at least three weeks, e.g. for 3, 4, 5, 6, 7, 8, 9 10 or more weeks (e.g. 20, 25, 30 or more weeks). This administration may be in a single cycle or in total in multiple cycles.

As referred to herein a “cycle” is a time period over which a particular treatment regime is applied and is generally repeated to provide cyclical treatment. The treatment in each cycle may be the same or different (e.g. different dosages, timings etc. may be used). In some embodiments, a cycle may be from 3-6 or 3-12 days in length, e.g. a 3, 4, 6, 9 or 12 day cycle. In some embodiments, a cycle may be about 1-6 weeks, i.e. daily administration for about 1-6, e.g. 1-4 or 1-3 weeks, such as about 1 or 2 weeks. In some embodiments, the cycle is repeated at least once. Thus, multiple cycles may be used, e.g. at least 2, 3, 4 or 5 cycles, e.g. 6, 7, 8, 9 or 10 (e.g. 10, 20, 30 or more) cycles.

In some embodiments, treatment cycles may be delimited by a break in treatment, e.g. a period without daily administration of the Cytor agonist and/or Tead1 antagonist. In some embodiments, the period between cycles is at least one day, e.g. 2, 3, 4 or more days. In some embodiments, the period between cycles is at least one week, e.g. 2, 3, 4 or more weeks.

However, in some embodiments, the second or subsequent treatment cycle may immediately follow the first or previous cycle. For instance, if the third daily dose of the first cycle was administered on day 3±1 day, the first daily dose of the second cycle may be administered on day 4±1 day.

In some embodiments of the invention, the patient may be subjected to other treatments prior to, contemporaneously with, or after the treatments of the present invention. For instance, in some embodiments, the patient may be treated with other procedures for the treatment of symptoms associated with the disease or condition that results in muscle fibre loss/muscle atrophy according to procedures known in the art.

In some embodiments, the Cytor agonist and/or Tead1 antagonist may be administered in combination with other therapeutic agents for the treatment of symptoms associated with the disease or condition that results in muscle fibre loss/muscle atrophy or other underlying condition, e.g. AIDS, CHF, COPD, cancer etc.

Thus, in some embodiments, the pharmaceutical composition containing the Cytor agonist and/or Tead1 antagonist may contain one or more additional therapeutic agents or may be for administration with one or more additional therapeutic agents.

The other therapeutic agents may be part of the same composition already comprising the Cytor agonist and/or Tead1 antagonist, in the form of a mixture, wherein the Cytor agonist and/or Tead1 antagonist and the other therapeutic agent are intermixed in or with the same pharmaceutically acceptable solvent and/or carrier or may be provided separately as part of a separate compositions, which may be offered separately or together in form of a kit of parts.

Thus, the Cytor agonist and/or Tead1 antagonist may be administered concomitantly with the other therapeutic agent separately, simultaneously or sequentially. For example, the Cytor agonist and/or Tead1 antagonist may be administered simultaneously with a first additional therapeutic agent or sequentially after or before administration of said first additional therapeutic agent. If the treatment regimen or schedule utilizes more than one additional therapeutic agent, the various agents may be partially administered simultaneously, partially sequentially in various combinations.

Thus, in some embodiments, the invention provides a Cytor agonist and/or Tead1 antagonist as defined herein in a combined product with another therapeutic agent for separate, simultaneous or sequential administration for use in treating or preventing a disease and/or condition associated with a loss of muscle fibres, particularly type II muscle fibres, or muscle atrophy.

Alternatively viewed, the method of the invention further comprises administering another therapeutic agent to said subject, wherein said therapeutic agent is administered separately, simultaneously or sequentially to the Cytor agonist and/or Tead1 antagonist as defined herein.

The therapeutic agents for use in combination with the Cytor agonist and/or Tead1 antagonist may be provided in pharmaceutical compositions as defined above and may be administered as defined above. Thus, the compositions comprising additional therapeutic agents may comprise pharmaceutically acceptable excipients, solvents and diluents suitable for such formulations.

The skilled person will be aware of suitable dosage ranges for any given additional therapeutic agent. In preferred embodiments, the additional therapeutic agent is present in the pharmaceutical composition, or administered to the subject, in its typical dose range.

The production of meat has a significant environmental impact and more efficient methods of meat production are desirable. Significant efforts have been dedicated to the production of “lab grown” meat in recent years.

As shown in the Examples, Cytor is a conserved lncRNA and Tead1 is a conserved transcription factor in animals. They have been shown to regulate myoblast differentiation into type II myoblasts. For instance, overexpression of Cytor in animals results in an increase in muscle mass. Accordingly, the inventors further propose modulation of Cytor (i.e. increase) and/or Tead1 (i.e decrease) expression in muscle cells (myoblasts) for the production of muscle fibres, e.g. for the in vitro production of meat. Moreover, modulation of Cytor and/or Tead1 expression in vivo, i.e. in non-human animals, is expected to result in animals with increased muscle mass and/or leaner meat.

Thus, a further aspect of the invention provides a modified non-human animal which has an increased amount of Cytor RNA and/or decreased amount of Tead1 RNA and/or protein in its skeletal muscle in comparison to a corresponding unmodified non-human animal.

In some embodiments the modified non-human animal has at least about a 1%, 2%, 3%, 4% or 5% increase in the amount of Cytor RNA and/or decrease in the amount of Tead1 RNA and/or protein in the skeletal muscle of the animal, such as about a 6%, 7%, 8%, 9%, 10% or more increase in the amount of Cytor RNA and/or decrease in the amount of Tead1 RNA and/or protein, e.g. about 12%, 15%, 20% or more, compared to a corresponding unmodified non-human animal.

The modified non-human animal may have an increased amount (e.g. size and/or number) of type II myotubes (muscle fibres) and/or an increased muscle mass compared to a corresponding unmodified non-human animal.

In some embodiments the modified non-human animal has at least about a 1%, 2%, 3%, 4% or 5% increase in the number and/or size of type II myotubes in the skeletal muscle of the animal, such as about a 6%, 7%, 8%, 9%, 10% or more increase in the number and/or size of type II myotubes, e.g. about 12%, 15%, 20% or more, compared to a corresponding unmodified non-human animal.

In some embodiments, the modified non-human animal has at least about a 1%, 2%, 3%, 4% or 5% increase muscle mass of the skeletal muscle of the animal, such as about a 6%, 7%, 8%, 9%, 10% or more increase muscle mass, e.g. about 12%, 15%, 20% or more, compared to a corresponding unmodified non-human animal.

In some embodiments, the modified non-human animal has been administered a Cytor agonist and/or Tead1 antagonist as defined above. In some embodiments, the Cytor agonist and/or Tead1 antagonist is a gene therapy.

Thus, in some embodiments, the modified non-human animal has been genetically-modified to increase the expression of Cytor RNA and/or decrease the expression of Tead1 RNA and/or protein.

Any suitable method of genetically-modifying the non-human animal to have increased Cytor RNA and/or decreased Tead1 RNA and/or protein may be used to produce the modified non-human animal. For instance, the endogenous Cytor promoter or enhancer may be modified or replaced to increase Cytor expression, e.g. the promoter or a part thereof, may be replaced with a heterologous promoter that results in increased expression of Cytor. Similarly, the endogenous Tead1 promoter or enhancer may be modified or replaced to decrease Tead1 expression, e.g. the promoter or a part thereof, may be replaced with a heterologous promoter that results in decreased expression of Tead1. In a further representative embodiment, the animal may be modified to include in its genome one or more additional copies of the Cytor gene or coding sequence and/or modified to mutate the Tead1 gene, so that a decreased amount of Tead1 RNA and/or protein is expressed.

Thus, in some embodiments, the modified non-human animal comprises in its genome (e.g. stably integrated into its genome), and is capable of expressing, one or more (e.g. one or two) nucleotide sequences encoding Cytor (as defined elsewhere herein), preferably wherein one nucleotide sequence is operably linked to a heterologous regulatory sequence (e.g. promoter). In some embodiments, the heterologous regulatory sequence (e.g. promoter) is a tissue-specific or cell-specific promoter, e.g. a muscle-specific (e.g. skeletal muscle-specific) or myoblast-specific promoter.

In a further aspect, the invention provides a method for producing a genetically-modified non-human animal as defined above. Methods of generating genetically modified non-human animals, e.g. from non-human pluripotent stem cells, are well-known in the art.

In a representative embodiment, the method comprises:
(i) providing a non-human cell (e.g. a non-human pluripotent stem cell or germ cell) which has been genetically-modified such that myoblast cells derived from said cell express an increased amount of Cytor RNA and/or decreased amount of Tead1 RNA and/or protein compared to unmodified myoblast cells; and
(ii) generating a genetically-modified non-human animal from said genetically-modified non-human cell (e.g. non-human pluripotent stem cell or germ cell).

Methods of producing non-human pluripotent stem cells, and genetically-modifying said cells, are known in the art and any suitable method may be used to provide the genetically-modified cell used in the method of the invention.

In some embodiments, the non-human pluripotent stem cell is a non-human pluripotent stem cell which comprises in its genome (e.g. stably integrated into its genome), one or more (e.g. one or two) nucleotide sequences encoding Cytor (as defined elsewhere herein), preferably wherein one nucleotide sequence is operably linked to a heterologous regulatory sequence (e.g. promoter). In some embodiments, the heterologous regulatory sequence (e.g. promoter) is a tissue-specific or cell-specific promoter, e.g. a muscle-specific (e.g. skeletal muscle-specific) or myoblast-specific promoter as defined above. In some related embodiments, the non-human pluripotent stem cell is a non-human pluripotent stem cell in which the Tead1 gene (one copy and preferably both copies) has been mutated, so that a decreased amount of Tead1 RNA and/or protein is expressed.

Typically and preferably, the step of generating a genetically-modified non-human animal of step (ii) comprises:
(a) introducing one or more non-human pluripotent stem cells of step (i) into a pre-implantation embryo of an animal of the same species;
(b) transferring said pre-implantation embryo into which one or more of said non-human pluripotent stem cells have been introduced in (a) into a female pseudo-pregnant non-human animal of the same species; and
(c) identifying a founder animal amongst the offspring of the female non-human animal of (b); and optionally
(d) mating said founder animal of (c) and identifying offspring thereof that have a genome the genetic modification that results in myoblast cells in said animal expressing an increased amount of Cytor RNA and/or decreased amount of Tead1 RNA and/or protein compared to unmodified myoblast cells.

In some embodiments, in step (a) the introduction of one or more non-human pluripotent stem cells into a pre-implantation embryo is done by injection.

In some embodiments, in step (d) a founder animal is typically mated with a non-founder animal of the same species. In some embodiments, in step (d), if the non-human pluripotent stem cells of (a) comprise a marker for positive selection flanked by site-specific recombination sites (e.g. loxP sites), the founder animal may be mated with an animal that expresses a site specific recombinase enzyme (e.g. Cre recombinase) in order to remove the marker for positive selection.

Typically, in step (d), the identifying of offspring comprises determining the genotype of the offspring to identify offspring that have a genome comprising the genetic modification that results in myoblast cells in said animal expressing an increased amount of Cytor RNA and/or decreased amount of Tead1 RNA and/or protein compared to unmodified myoblast cells. Alternatively viewed, in step (d) the identifying of offspring comprises identifying those offspring that result from germline transmission of genomic DNA of the non-human pluripotent stem cell of (a), said genomic DNA comprising the genetic modification that results in myoblast cells in said animal expressing an increased amount of Cytor RNA and/or decreased amount of Tead1 RNA and/or protein compared to unmodified myoblast cells.

In some embodiments, determining the genotype of the offspring is performed by a PCR-based method and/or by DNA sequencing. Suitable methods are known in the art.

In some embodiments, the genetically modified non-human animal produced by the method of the invention is heterozygous for the genetic modification that results in myoblast cells in said animal expressing an increased amount of Cytor RNA and/or decreased amount of Tead1 RNA and/or protein compared to unmodified myoblast cells.

In some embodiments, the genetically modified non-human animal produced by the method of the invention is homozygous for the genetic modification that results in myoblast cells in said animal expressing an increased amount of Cytor RNA and/or decreased amount of Tead1 RNA and/or protein compared to unmodified myoblast cells.

Producing non-human animals that are homozygous for the genetic modification that results in myoblast cells in said animal expressing an increased amount of Cytor RNA and/or decreased amount of Tead1 RNA and/or protein compared to unmodified myoblast cells may be done by mating together non-human animals that are heterozygous for the genetic modification and identifying (e.g. by PCR-based genotyping) amongst the offspring of such a mating those animals that are homozygous for the genetic modification.

Thus, in some embodiments, the method of producing (or generating) a genetically modified non-human animal of the invention further comprises steps of identifying non-human animals produced as being heterozygous for the genetic modification in accordance with the invention, mating such heterozygous non-human animals together, and identifying offspring from said mating that are homozygous for the genetic modification in accordance with the invention. Such a method thereby produces a genetically modified non-human animal in accordance with the invention that is homozygous for the genetic modification in accordance with the invention.

The modified (e.g. genetically-modified) non-human animal of the invention may be a domesticated or farmed animal, e.g. livestock (such as poultry, pigs, cattle, sheep or goats) or farmed fish (e.g. salmon, tilapia or tuna).

In a further aspect, the invention provides an in vitro method for producing muscle fibres (e.g. type II myotubes or comprising type II myotubes) comprising culturing modified myoblasts which have increased amounts of Cytor RNA and/or decreased amount of Tead1 RNA and/or protein in comparison to unmodified myoblasts under conditions suitable to produce muscle fibres.

In some embodiments, the modified myoblasts have been administered (e.g. contacted with) a Cytor agonist and/or a Tead1 antagonist as defined above. In some embodiments, the Cytor agonist and/or a Tead1 antagonist is a gene therapy.

Thus, in some embodiments, the modified myoblasts have been genetically-modified to increase the expression of Cytor RNA and/or decrease the expression of Tead1 RNA and/or protein.

Any suitable method of genetically-modifying the myoblasts to have increased Cytor RNA and/or decreased Tead1 RNA and/or protein may be used to produce the modified myoblasts. For instance, the endogenous Cytor promoter or enhancer may be modified or replaced to increase Cytor expression, e.g. the promoter or a part thereof, may be replaced with a heterologous promoter that results in increased expression of Cytor. Similarly, the endogenous Tead1 promoter or enhancer may be modified or replaced to decrease Tead1 expression, e.g. the promoter or a part thereof, may be replaced with a heterologous promoter that results in decreased expression of Tead1. In a further representative embodiment, the myoblasts may be modified to include in its genome one or more additional copies of the Cytor gene and/or modified to mutate the Tead1 gene, so that a decreased amount of Tead1 RNA and/or protein is expressed. It is also possible to add one or more additional copies of the Cytor gene via an expression vector that result in the ectopic expression of Cytor. Ectopic expression refers to the occurrence of gene expression in a tissue in which it is normally not expressed and this may be achieved, for example, by placing the one or more additional copies of the Cytor gene under the control of tissue-specific promoter.

In some embodiments, the genetically-modified myoblasts are obtained indirectly. For instance, pluripotent stem cells (e.g. non-human cells) may be genetically-modified and subsequently cultured to produce myoblast cells that contain the genetic modification.

Thus, in some embodiments, the modified myoblasts comprise in their genome (e.g. stably integrated into their genome), and are capable of expressing, one or more (e.g. one or two) nucleotide sequences encoding Cytor (as defined elsewhere herein), preferably wherein one nucleotide sequence is operably linked to a heterologous regulatory sequence (e.g. promoter). In some embodiments, the heterologous regulatory sequence (e.g. promoter) is a tissue-specific or cell-specific promoter, e.g. a muscle-specific (e.g. skeletal muscle-specific) or myoblast-specific promoter as defined above.

Methods of generating genetically-modified myoblasts are well-known in the art and any suitable method may be used in the methods of the invention.

In some embodiments, method may involve introducing a vector comprising a nucleotide sequence encoding Cytor (as defined herein), wherein the vector is capable of transfecting or transducing a cell (e.g. myoblast), such that the cell or a myoblast cell derived therefrom expresses the Cytor RNA.

The vector may be a non-viral vector such as a plasmid. Plasmids may be introduced into cells (e.g. myoblasts) using any well-known method of the art, e.g. using calcium phosphate, liposomes, or cell penetrating peptides (e.g. amphipathic cell penetrating peptides).

The vector may be a viral vector, as described above, such as a retroviral, e.g. a lentiviral vector or a gamma retroviral vector.

Vectors suitable for delivering nucleic acids for expression in mammalian cells are well-known in the art and any such vector may be used. Vectors may comprise one or more regulatory elements, e.g. a promoter, such as a tissue- or cell-specific promoter as defined above.

Delivery systems are also available in the art which do not rely on vectors to introduce a nucleic acid molecules into a cell, for example, systems based on transposons, CRISPR/TALEN delivery and mRNA delivery. Any such system can be used to deliver a nucleic acid molecule into a cell according to the present invention.

In a further aspect, the invention provides meat or a meat-containing product derived or obtained from the modified non-human animal of the invention.

In another aspect, the invention provides muscle fibres and/or meat produced by the in vitro method of the invention.

It will be evident that the in vitro method of the invention advantageously may be used to provide muscle fibres and/or muscle tissue for use in therapy, e.g. in transplants, and this use forms a further aspect of the invention. Thus, in some embodiments, the modified myoblasts may be human or non-human animal myoblasts.

However, in some embodiments, muscle fibres and/or meat produced by the in vitro method of the invention may be for human or non-human animal consumption. Accordingly, in these embodiments, the myoblasts are non-human animal myoblasts, such as myoblasts from a domesticated or farmed animal, e.g. livestock (such as poultry, pigs, cattle, sheep or goats) or farmed fish (e.g. salmon, tilapia or tuna).

Meat products or meat-containing products of the invention (i.e. non-human meat) may include processed meat products, which may be for human or non-human animal consumption, e.g. pet food. The meat may be combined with other components, e.g. fats, oils, lipids, carbohydrates, protein supplements, fibre etc. to obtain a meat product or meat-containing product suitable for consumption.

As discussed above, the inventors have identified a cis-eQTL that is involved in the regulation of Cytor expression. Accordingly, the inventors propose that this cis-eQTL may find utility in various diagnostic and prognostic tests relating to muscle mass and muscle function, particularly type II muscle fibre mass and function.

Accordingly, in a further aspect, the invention provides a method of genotyping a subject (i.e. a human subject) for the cis-eQTL rs74360724, i.e. determining whether the subject is homozygous for the G allele or A allele, or heterozygous, and/or cis-eQTL rs 79200838, i.e. determining whether the subject is homozygous for the C allele or T allele, or heterozygous.

More particularly, the invention provides a method for determining the risk of developing skeletal muscle atrophy (e.g. sarcopenia) in a subject comprising determining the genotype of the subject at the cis-eQTL rs74360724, wherein when the subject is heterozygous or homozygous for the G allele they have an increased risk of developing muscle atrophy (e.g. sarcopenia) compared to a subject that is homozygous for the A allele, and/or at the cis-eQTL rs79200838, wherein when the subject is heterozygous or homozygous for the T allele they have an decreased risk of developing muscle atrophy (e.g. sarcopenia) compared to a subject that is homozygous for the C allele.

In another aspect, the invention provides a method for predicting the performance of a subject in an activity associated with fast-twitch muscle (e.g. an athlete, e.g. a sprinter) comprising determining the genotype of the subject at the cis-eQTL rs74360724, wherein when the subject is heterozygous or homozygous for the A allele they have an increased likelihood of outperforming a subject that is homozygous for the G allele, and/or at the cis-eQTL rs79200838, wherein when the subject is heterozygous or homozygous for the C allele they have an decreased of outperforming a subject that is homozygous for the T allele.

In a still further aspect, the invention provides a method for predicting the capability of a subject to produce fast-twitch muscle comprising determining the genotype of the subject at the cis-eQTL rs74360724, wherein when the subject is heterozygous or homozygous for the A allele they have an increased capability of producing fast-twitch muscle compared a subject that is homozygous for the G allele, and/or at the cis-eQTL rs79200838, wherein when the subject is heterozygous or homozygous for the C allele they have an decreased capability of producing fast-twitch muscle compared a subject that is homozygous for the T allele.

Methods of genotyping subjects are well-known in the art and any suitable method may be used in the methods described above. In some embodiments, the method involves isolating genomic DNA from a sample obtained from the subject, e.g. a cell containing sample such as a blood sample, cheek swab etc. and analysing the DNA to determine the genotype, e.g. using PCR-based methods.

The invention will now be further described with reference to the following non-limiting Examples and Figures in which:

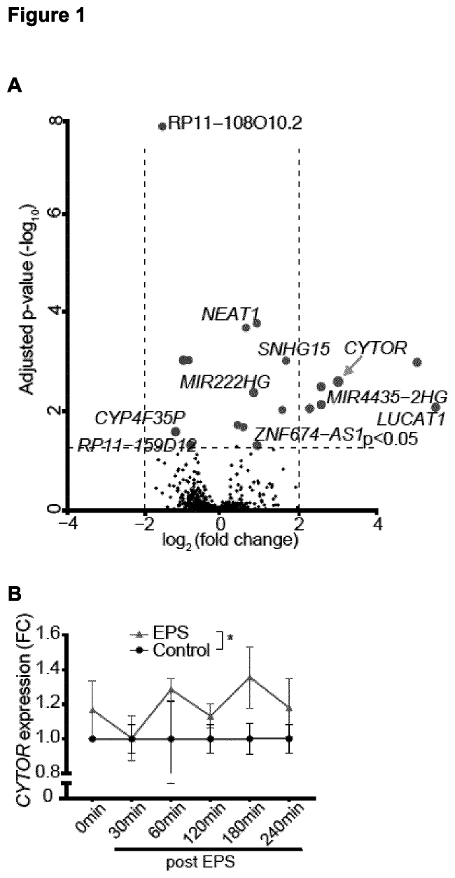

FIG. 1 shows (A) a volcano plot of differentially expressed lncRNAs in human vastus lateralis 3 h after one bout of leg extension exercise (GSE71972; n=8 humans per group); and (B) CYTOR mRNA levels at several timepoints after electrical pulse stimulation in differentiated human skeletal myotubes (n=4 replicates). All data show mean±SEM. *P<0.05, ****P<0.0001 two-way ANOVA (B).

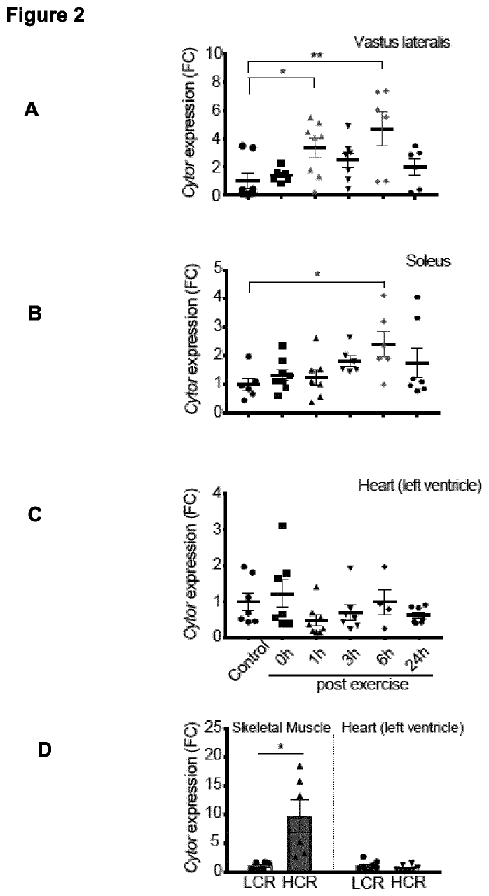

FIG. 2 shows (A)-(C) Cytor mRNA expression after incremental uphill treadmill exercise in rat (A) vastus lateralis, (B) soleus and (C) left ventricle (n=6-8 rats per group); and (D) Cytor mRNA in skeletal muscle and left ventricle of rats inbred (34 generations) for low- (LCR) and high-aerobic running (HCR) capacity (n=6-8 rats per group). All data show mean±SEM. *P<0.05, **P<0.01 Student's two-tailed t test.

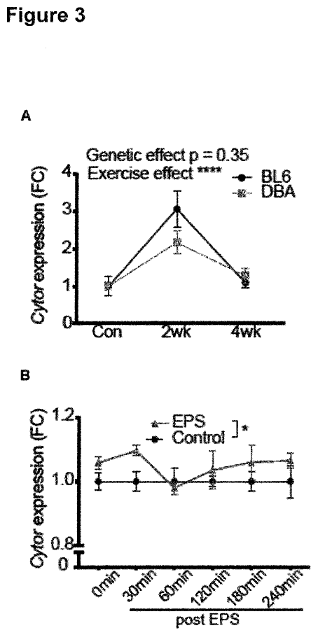

FIG. 3 shows (A) Cytor mRNA expression upon exercise training in skeletal muscle of C57BL/6JRj and DBA/2J mice strains (n=4 mice per group); and (B) CYTOR mRNA levels at several time points after electrical pulse stimulation in differentiated mouse C2C12 myotubes (n=3 replicates). All data show mean±SEM. *P<0.05, ****P<0.0001 two-way ANOVA.

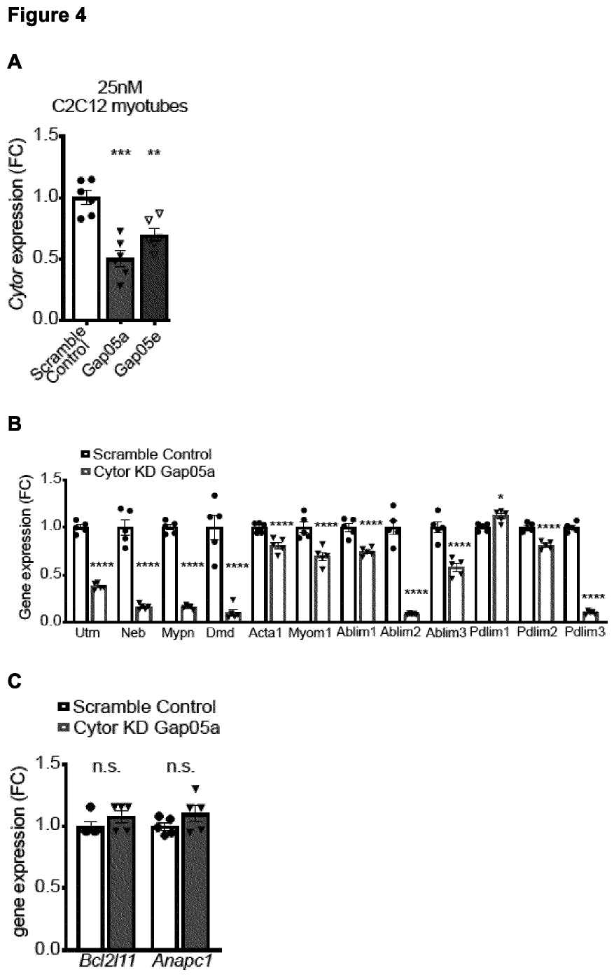

FIG. 4 shows (A) Cytor expression in differentiated C2C12 myotubes 24 h after transfection of a scramble Gapmer (Scramble Control) or a Gapmer targeting Cytor (Gap05a, Gap05e) (n=6 per group); (B) Normalized gene expression of muscle structure genes in control versus Cytor-deficient C2C12 myotubes (n=4 replicates per group); and (C) Gene expression upon Gapmer-mediated knockdown (Gap05a) of Cytor in C2C12 cells (n=5 per group). All data show mean±SEM. **P <0.01, ***P<0.001 Student's two-tailed t test.

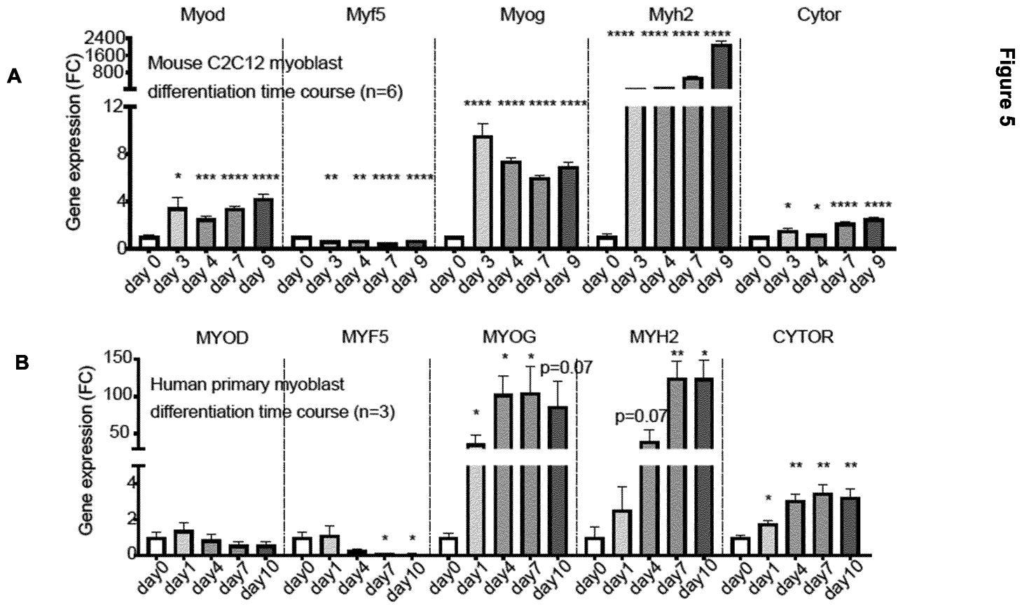

FIG. 5 shows (A-B) normalized gene expression in differentiating (A) C2C12 myoblasts and (B) human primary myoblasts (n=3-6 replicates per group). All data show mean±SEM. *P<0.05, **P<0.01, ****P<0.0001 Student's two-tailed t test.

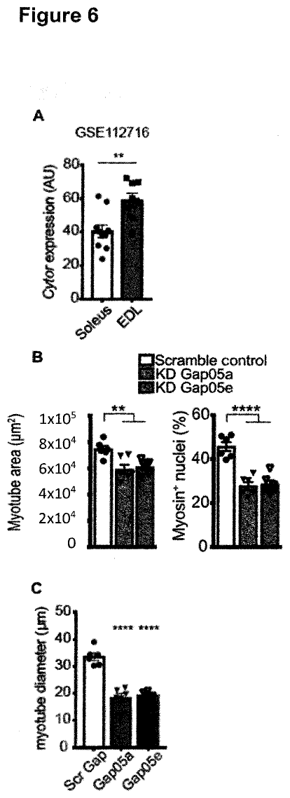

FIG. 6 shows (A) Cytor expression in Soleus (n=10) and EDL (n=8) muscles in mice (GSE112716) representing type I and type II muscle, respectively; (B) Quantification of myotube area (left) and myofusion index (right) calculated as fraction of nuclei present within myotubes in Cytor knockdown C2C12 (n=6 replicates); and (C) Gene expression of C2C12 Myotube thickness of horse serum differentiated C2C12 myotubes 48 h after Gapmer-mediated Cytor knockdown (n=6 per group). All data show mean±SEM. *P<0.05, **P<0.01, ****P<0.0001 Student's two-tailed t test.

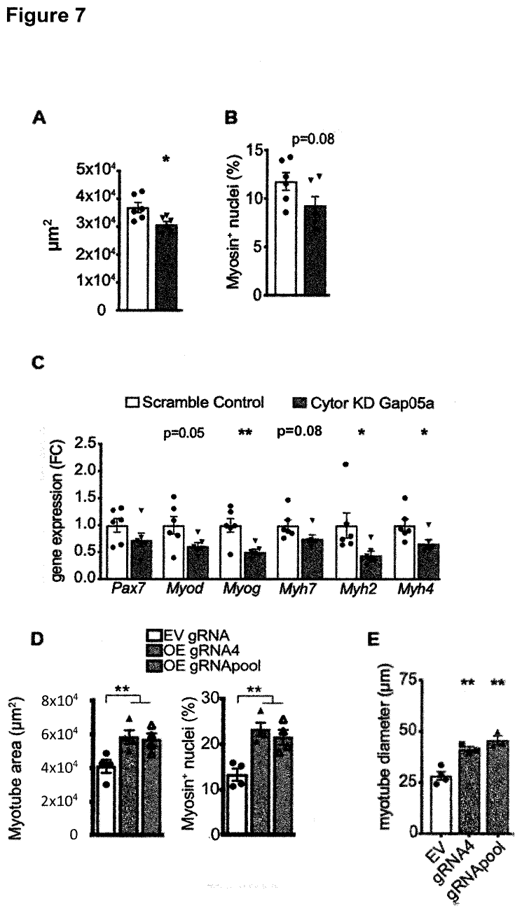

FIG. 7 shows (A) Myotube area, (B) Myofusion index and (C) normalized gene expression of differentiating C2C12 myoblasts upon Cytor knockdown (n=6 replicates per group); (D) Quantification of myotube area (left) and myofusion index (right) calculated as fraction of nuclei present within myotubes in Cytor overexpressing C2C12 cells; and (E) Myotube thickness of C2C12 myoblasts after spCRISPRa mediated Cytor overexpression (n=3-4 per group). All data show mean±SEM. *P<0.05, **P<0.01, ***P<0.001, ****P<0.0001 Student's two-tailed t test.

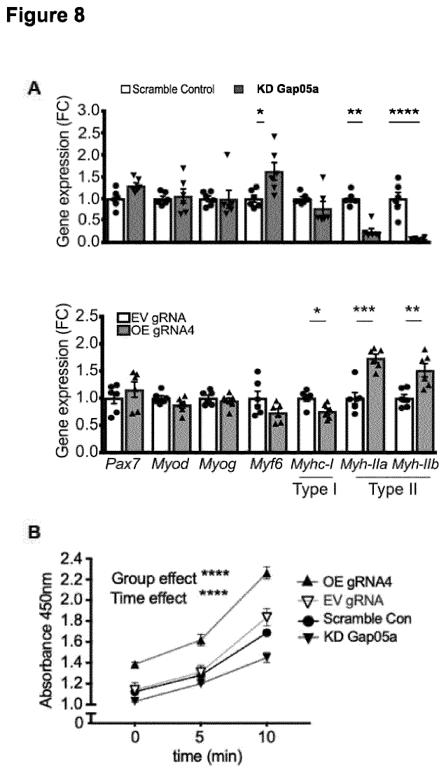

FIG. 8 shows (A—top) Gene expression of C2C12 myotubes transfected with scramble control or Gap05 (fig. S2G) to knockdown Cytor (n=6); (A—bottom) Gene expression of C2C12-spdCas9-VP64-MPH expressing myoblasts transfected with gRNA4 to overexpress Cytor (bottom, n=6); and (B) Phosphofructokinase activity in control versus Cytor knockdown or Cytor overexpressing C2C12 myoblasts. All data show mean±SEM. *P<0.05, **P<0.01, ***P<0.001, ****P<0.0001 Student's two-tailed t test.

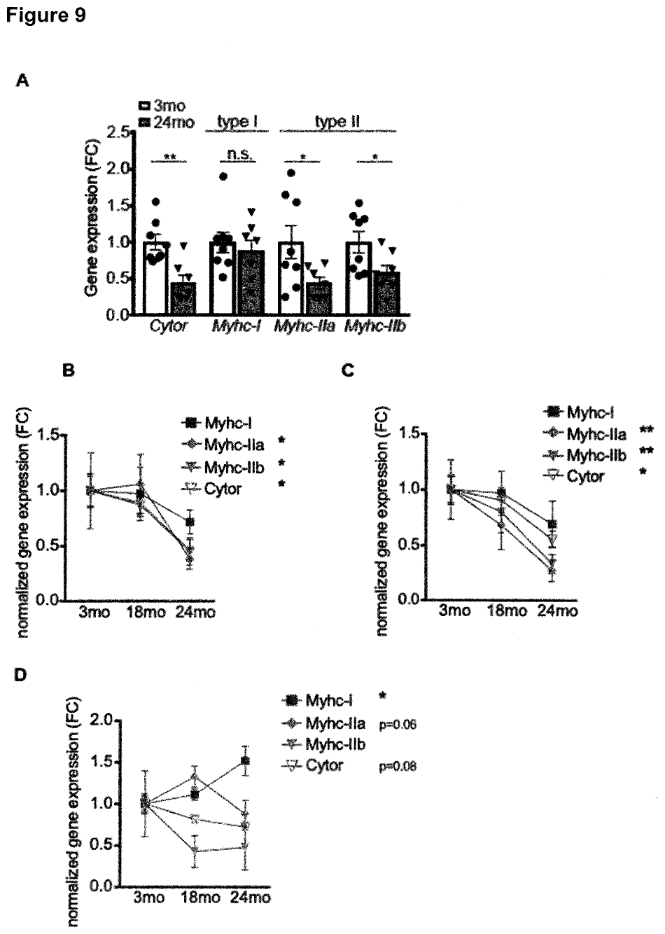

FIG. 9 shows results relating to experiments in which right and left gastrocnemius muscle of 3 mo old mice injected with a scramble control or Gap05a to knockdown Cytor (n=9 mice per group). 18 mo mice were intramuscularly injected with adeno associated virus containing empty vector sadCas9-VP64-U6 or sadCas9-VP64-U6-gRNA3 (n=8-10 mice per group). (A) mRNA transcript abundance of Cytor, Myhc-I (Myh7), Myhc-IIa (Myh2) and Myhc-IIb (Myh4) in gastrocnemius muscle of 3 mo and 24 mo non-injected mice (n=7-8 mice per group); (B-D) normalized gene expression of slow- and fast myosin isoforms and Cytor in mouse (B) Gastrocnemius, (C) EDL and (D) Soleus muscle. All data show mean±SEM. *P<0.05, **P<0.01, ***P<0.001, ****P<0.0001 Student's two-tailed t test (A). ****P<0.0001 two-way ANOVA (H). *P<0.05, **P<0.01 (B-D).

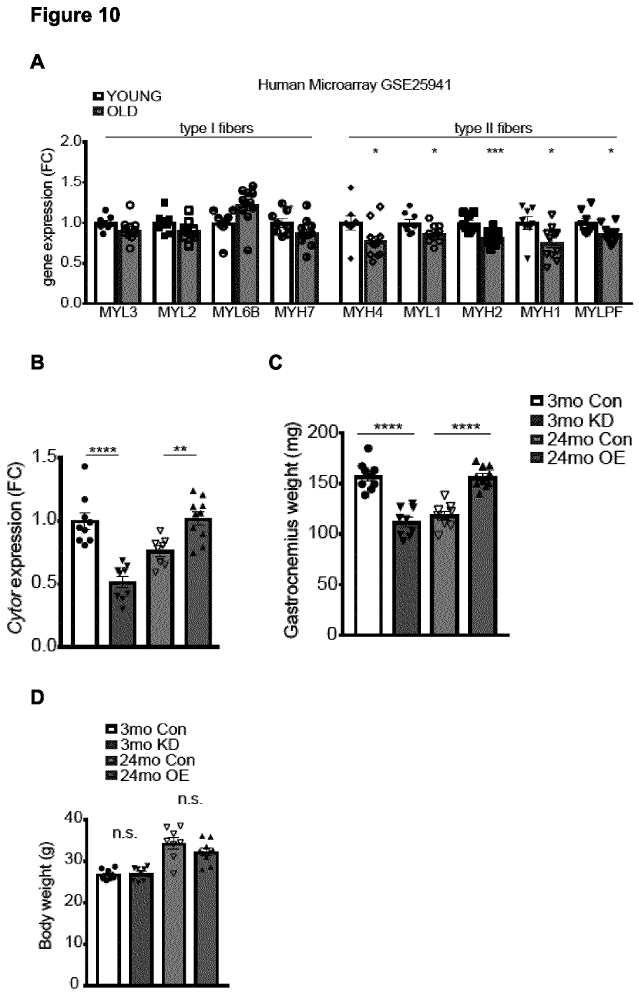

FIG. 10 shows (A) Expression of genes encoding type I and type II muscle

fibers in human skeletal muscle from young (n=8 humans) and old females (n=11 humans) (GSE25941); (B) Cytor expression in gastrocnemius muscle and (D) body weight of 5 months-old mice intramuscularly injected with a scramble control Gapmer or a Gapmer targeting Cytor (n=9 mice per group) and in 24 mo old mice intramuscularly injected with a non-targeting AAV-sadCas9-VP64-U6-gRNA (n=8 mice) or AAV-sadCas9-VP64-U6-gRNA3 targeting the promoter of Cytor (n=8-10 mice per group); and (C) Gastrocnemius muscle weight from hindlimb (n=8-10 mice per group). All data show mean±SEM. *P<0.05, **P<0.01, ***P<0.001, ***P<0.0001 Student's two-tailed t test.

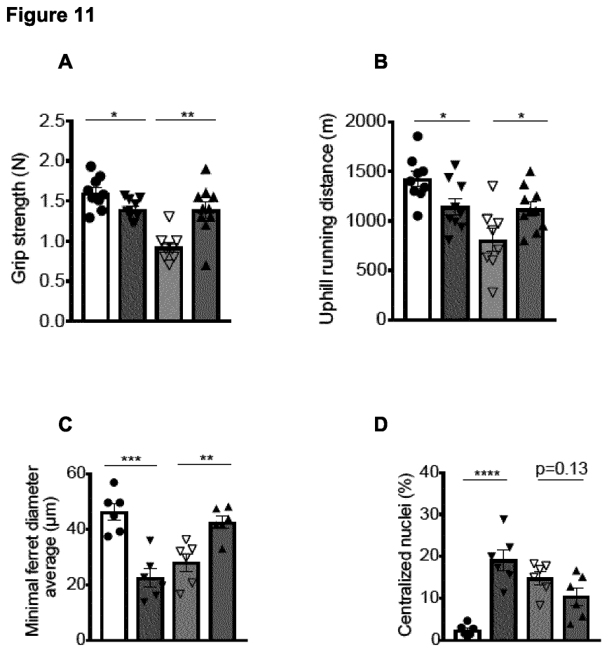

FIG. 11 shows (A) Hindlimb grip strength (n=8-10 mice per group); (B) maximal uphill (10⁰) running distance (n=8-10 mice per group); (C) Quantification of average fiber minimal feret diameter (n=6 mice per group); and (D) proportion of muscle fibers presenting centralized nuclei (n=6 mice per group). All data show mean±SEM. *P<0.05, **P<0.01, ***P<0.001, ***P<0.0001 Student's two-tailed t test.

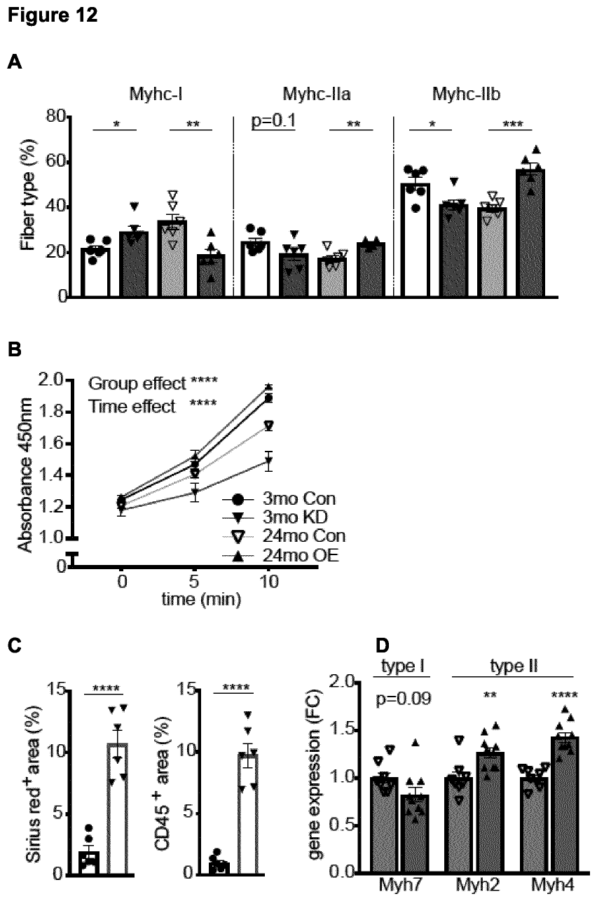

FIG. 12 shows (A) Quantification of distribution of type II muscle fibers stained for laminin, Myhc-I (Myh7), Myhc-IIa (Myh2) and Myhc-IIb (Myh4) in gastrocnemius muscle (n=6 mice per group); (B) phosphofructokinase activity of 5 months-old mice intramuscularly injected with a scramble control Gapmer or a Gapmer targeting Cytor (n=9 mice per group) and in 24 mo old mice intramuscularly injected with a non-targeting AAV-sadCas9-VP64-U6-gRNA (n=8 mice) or AAV-sadCas9-VP64-U6-gRNA3 targeting the promoter of Cytor (n=8-10 mice per group); (C) Quantification of Sirus red and CD45 staining of 5 months-old mice intramuscularly injected with a scramble control Gapmer or a Gapmer targeting Cytor (n=6 mice per group); and (D) normalized gene expression in 24 mo old mice intramuscularly injected with a non-targeting AAV-sadCas9-VP64-U6-gRNA (n=8 mice) or AAV-sadCas9-VP64-U6-gRNA3 targeting the promoter of Cytor (n=10 mice per group). All data show mean±SEM. *P<0.05, **P<0.01, ***P<0.001, ***P<0.0001 Student's two-tailed t test. ****P<0.0001 two-way ANOVA (B).

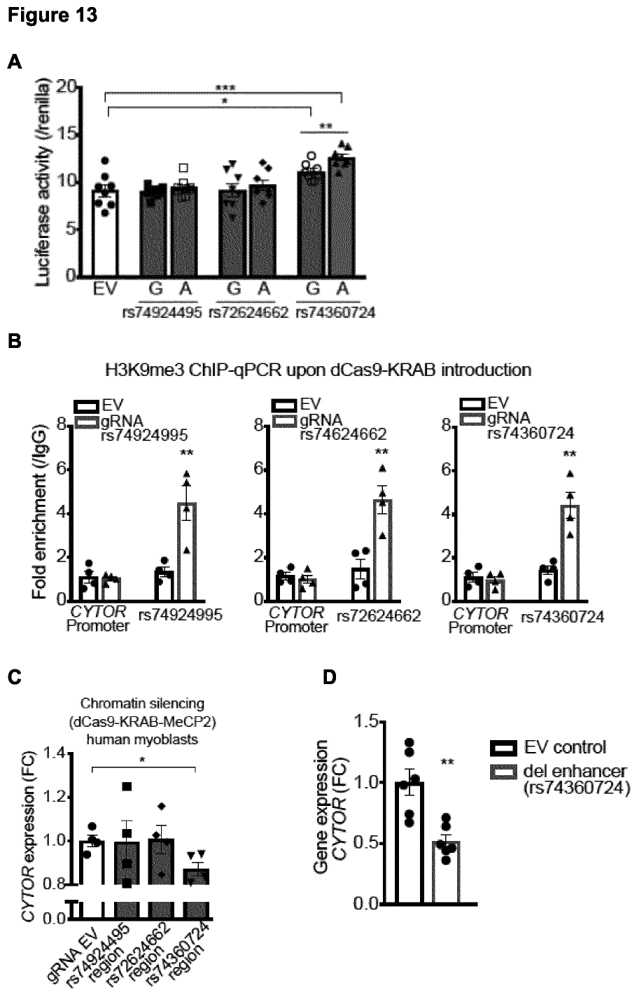

FIG. 13 shows (A) Renilla-normalized luciferase activity in human skeletal muscle myoblasts expressing the pGL3-Promoter vector without (EV) or with a 25 bp fragment surrounding the putative CYTOR enhancer elements rs74360724, rs74924495, rs72624662 (n=7-8 replicates per variant); (B) Chromatin immunoprecipitation of H3K9me3 followed by qPCR amplification of rs74924995, rs72624662 and rs74360724 in human primary myoblasts co-expressing dCas9-KRAB-MeCP2 with an empty vector guide RNA or a gRNA targeting the indicated genomic regions (n=4 replicates per group); (C) CYTOR expression in human skeletal muscle myoblasts expressing dCas9-KRABMeCP2 and an empty vector gRNA (EV) or a gRNA targeting the putative CYTOR enhancer elements rs74360724, rs74924495, or rs72624662 (n=4 replicates per variant); (D) Normalized CYTOR gene expression in human primary myoblasts expressing Cas9 (EV), or Cas9-gRNAs flanking rs74360724 (n=6 replicates per group). All data show mean±SEM. *P<0.05, **P<0.01, ***P<0.001 Student's two-tailed t test.

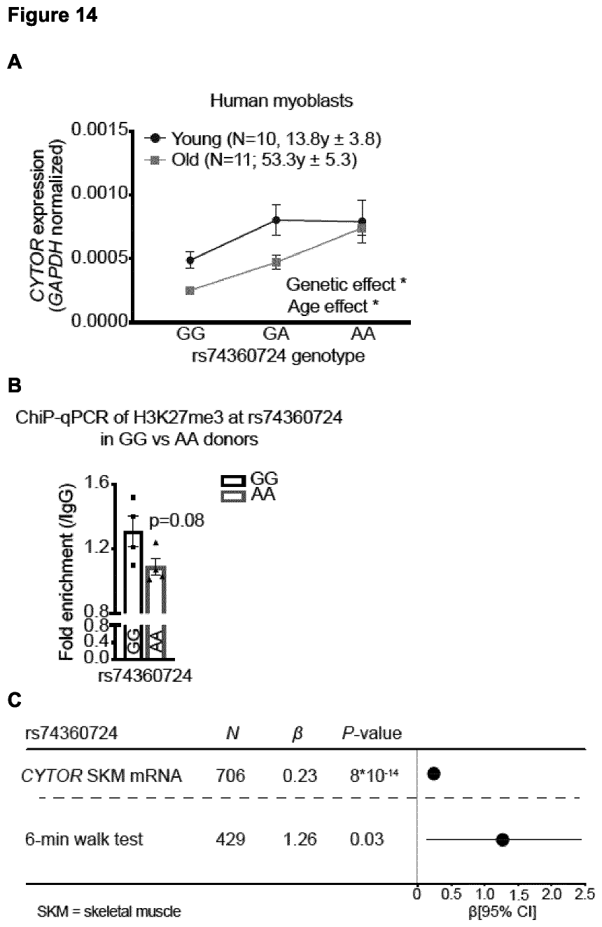

FIG. 14 shows (A) CYTOR expression in human myoblasts from 21 donors of different ages and genotypes for rs74360724 (n=5 GG, n=11 GA, n=5 AA); (B) Chromatin immunoprecipitation of H3K27me3 followed by qPCR of rs74360724 in human primary myoblasts from donors homozygous for GG or AA at rs74360724; (C) Effect of rs74360724 on CYTOR skeletal muscle mRNA from the human GTEx project and performance in 6-min walk test in aged individuals from the Helsinki Birth Cohort Study (HBCS) (n=706 humans for mRNA expression; n=429 humans for 6-min walk test). All data show mean±SEM. *P<0.05 two-way ANOVA.

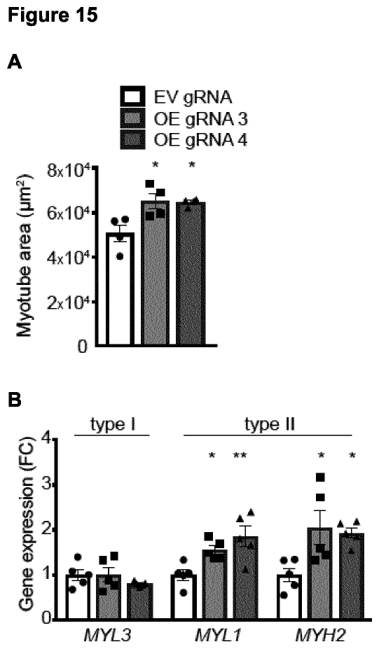

FIG. 15 shows (A) Quantification of stained mature myotubes of aged human myoblasts (76 years) expressing spdCas9-VP64 without (EV gRNA) or with a gRNA (OE gRNA3; OE gRNA4) to overexpress CYTOR endogenously: myotube area (n=4 replicates); and (B) Gene expression of type I and type II myosin isoforms in aged human myoblasts (76 years) expressing spdCas9-VP64 without (EV gRNA) or with a gRNA (OE gRNA3; OE gRNA4) to overexpress CYTOR endogenously. All data show mean±SEM. *P<0.05, **P<0.01, Student's two-tailed t test.

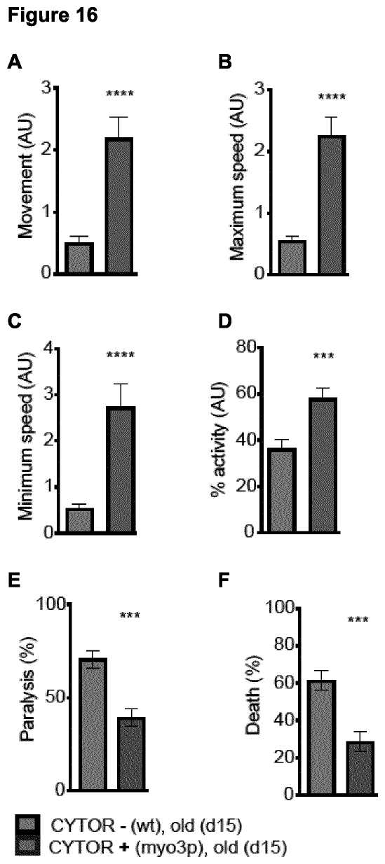

FIG. 16 shows (A) Movement (n=38 CYTOR −, n=68 CYTOR +), (B) maximum speed (n=42 CYTOR −, n=72 CYTOR +), (C) minimum speed (n=42 CYTOR −, n=72 CYTOR +), (D) fraction of active worms (n=42 CYTOR −, n=72 CYTOR +), (E) fraction of paralyzed worms (n=10 worms per group) and (F) fraction of dead worms (n=10 worms per group) at day 15 in N2 C. elegans strains without (CYTOR −) or with (CYTOR +) incorporation of human CYTOR DNA under the muscle-specific promoter myo3p. All data show mean±SEM. ***P<0.001, ****P<0.0001 Student's two-tailed t test.

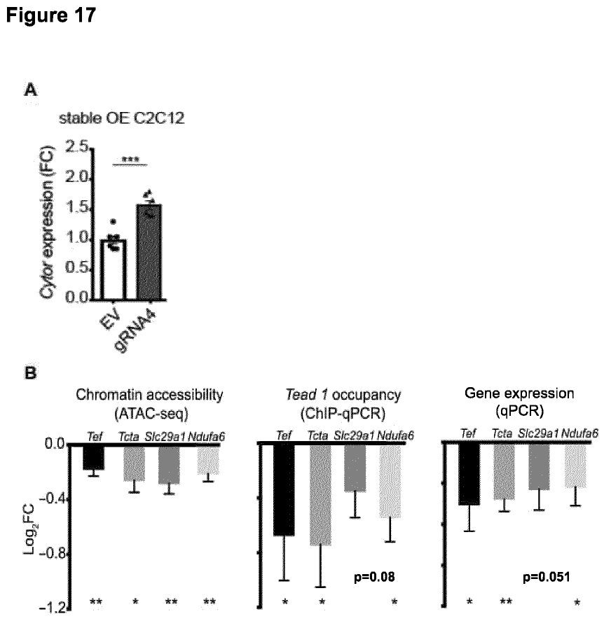

FIG. 17 shows (A) Cytor expression in C2C12 myoblasts stably expressing spdCas9-VP64-gRNA4; and (B) Changes in ATACseq signal (left, n=4 replicates per group), Tead1 occupancy (middle, n=4 replicates per group), and gene expression (right, n=6 replicates per group) in C2C12 overexpressing Cytor. All data show mean±SEM. *P<0.05, **P<0.01, ***P<0.001, ****P<0.0001 Student's two-tailed t test.

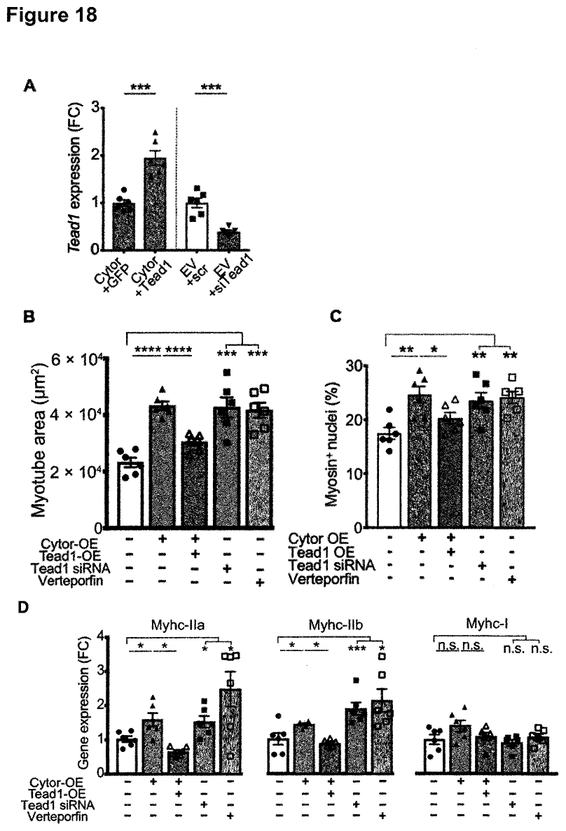

FIG. 18 shows (A) Normalized Tead1 gene expression in Cytor overexpressing C2C12 cells transiently transfected with a Tead1- or GFP-expressing plasmid (left) and Tead1 expression in empty vector control C2C12 cells transiently transfected with a scramble siRNA or siRNA against Tead1 (right) (n=6 replicates per group); (B) Quantification of myotube area and (C) myofusion index of C2C12 myoblasts overexpressing Cytor alone, together with Tead1, or C2C12 myoblasts treated with siRNA against Tead1 or Verteporfin (n=6 replicates); and (D) Gene expression in C2C12 myoblasts overexpressing Cytor alone, together with Tead1 or treated with siRNA against Tead1 or Verteporfin (n=6 replicates). All data show mean±SEM. *P<0.05, **P<0.01, ***P<0.001, ****P<0.0001 Student's two-tailed t test.

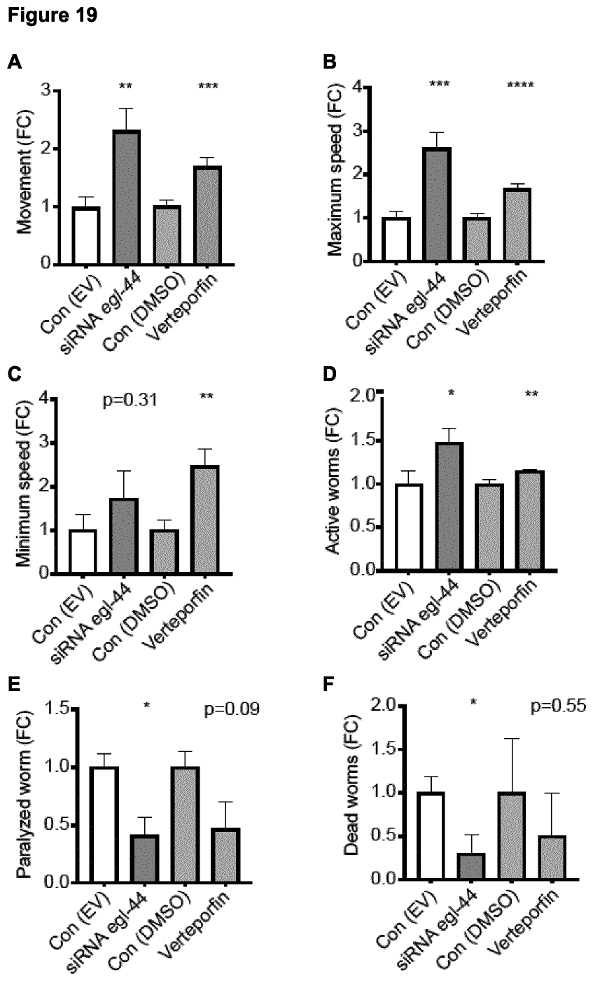

FIG. 19 shows (A) Movement (n=51 Con-EV, n=48 siRNA egl-44, n=48 Con-DMSO, n=47 Verteporfin), (B) maximum speed (n=51 Con-EV, n=48 siRNA egl-44, n=48 Con-DMSO, n=47 Verteporfin), (C) minimum speed (n=51 Con-EV, n=48 siRNA egl-44, n=49 Con-DMSO, n=54 Verteporfin) (D) fraction of active worms (n=48 Con-EV, n=51 siRNA egl-44, n=48 Con-DMSO, n=47 Verteporfin) (E) fraction of paralyzed worms (n=6 replicates per group) and (F) fraction of dead worms (n=6 replicates per group). All data show mean±SEM. *P<0.05, **P<0.01, ***P<0.001, ****P<0.0001 Student's two-tailed t test.

FIG. 20 shows (A) Allelic effect of the variant rs79200838 on TEAD1 expression in the Genotype-Tissue Expression (GTEx) database (n=553 for CC; n=31 for CT), (B) phenotype association of rs79200838 in the UK biobank, (C) effect size and p-value of the TEAD1 expression-reducing T-allele at rs79200838 for the top 6 affected phenotypes in the UK biobank.

### EXAMPLES

### Example 1: Exercise Alters Expression of lncRNAs in Skeletal Muscle

To discover lncRNAs potentially having major impact on skeletal muscle function, we analyzed differentially expressed lncRNAs after single-leg knee-extension exercise in an extant human skeletal muscle dataset (GSE71972) and identified several lncRNAs differentially expressed after exercise (FIG. 1A). When ranking these lncRNAs based on their responsiveness to exercise in both effect size and statistical significance, the lncRNA with highest responsiveness to exercise was identified to be CYTOR (Table 1).

TABLE 1
IncRNAs altered by exercise in human skeletal muscle

| | q-value | log2FC | q-value | log2FC | Average | Average | Average |
|---|---|---|---|---|---|---|---|
| IncRNA | Kallisto | Kallisto | STAR | STAR | log2FC | q-value | Rank |
| CYTOR | 2.41E−03 | 3.0126 | 1.91E−03 | 2. [symbol] 141 | 2.9134 | 2.16E−03 | 3 |
| SNHG15 | 9.00E−04 | 1.6749 | 5.32E−04 | 1.6571 | 1. [symbol] 0 | 7.16E−04 | 4 |
| MIR4435-2HG | 7.18E−03 | 2.5770 | 8.64E−04 | 1.9771 | 2.2770 | 4.02E−03 | 5.5 |
| CTD-3252C9.4 | 1.54E−04 | 0.92 [symbol] 3 | 3.5 [symbol] E−03 | 1.0181 | 0.9732 | 1. [symbol] 7E−03 | 5.5 |
| RP11-309L24.4 | 9.73E−04 | 5.0435 | 1. [symbol] E−02 | 3.0152 | 4.0293 | 9.77E−03 | 6.5 |
| MIR222HG | 3.0 [symbol] E−03 | 2.5740 | 1.07E−02 | 2.2389 | 2.4065 | [symbol] E−03 | 6.5 |
| LUCAT1 | [symbol] .16E−03 | 5.5184 | 2.23E−02 | 4.1693 | 4.8438 | 1.52E−02 | 7 |
| AC058791.1 | 8.62E−03 | 2.2 [symbol] 28 | 5.37E−03 | 1. [symbol] 900 | 2.0864 | [symbol] .99E−03 | 8 |
| RP1-206D16.6 | 9.18E−03 | 1.5 [symbol] 23 | 9.28E−04 | 1.7 [symbol] 00 | 1. [symbol] 562 | 5.05E−03 | 8 |
| BPGBPG55C20.2 | 8.78E−04 | −0.8229 | 5.37E−03 | −0.8084 | −0.8156 | 3.13E−03 | 8.5 |
| MIR22HG | 4.05E−03 | 0.843 [symbol] | 5.37E−03 | 0.9279 | 0. [symbol] 857 | 4.71E−03 | 9.5 |
| RP11-159D12.2 | 8.78E−04 | −0.9 [symbol] 03 | [symbol] .26E−03 | −0.8460 | −0.9032 | 3. [symbol] 7E−03 | 9.5 |
| NEAT1 | 1.04E−02 | 0.6134 | 1. [symbol] 7E−02 | 1.1928 | 0.9031 | 1.4 [symbol] E−02 | 11.5 |
| ZNF674-AS1 | 4.90E−02 | 0.9343 | 1.07E−02 | 0.9641 | 0.9492 | 2.9 [symbol] E−02 | 12.5 |
| CYP4F35P | 2.59E−02 | −1.1637 | 1. [symbol] 7E−02 | −0.9028 | −1.0332 | 2.23E−02 | 14.5 |

[symbol]

indicates data missing or illegible when filed

Increased CYTOR expression upon resistance exercise, and to some degree upon endurance exercise was confirmed in a range of public available datasets. CYTOR has been linked to breast, gastric, and colon cancers and possesses a second copy in the human genome (MIR4435-2HG), which was more recently described as Morrbid. CYTOR is not in close proximity (>100 kb) to other genes and shows nucleotide conservation in mice (annotated as Gm14005) and rats (annotated as XR_146885.3), yet there is no previous literature on the role of CYTOR/Cytor in muscle physiology. Electrical pulse stimulation (EPS) to model exercise in cells, confirmed CYTOR upregulation in human muscle cells (FIG. 1B) suggesting that the induction of CYTOR by exercise is regulated in a cell autonomous manner. RNA fluorescence in situ hybridization (RNA-FISH) in human myoblasts revealed CYTOR to be mainly localized in the nucleus. This was confirmed by subcellular fractionation experiments in mouse myoblasts.

Given the presence of Cytor homologues in rats and mice, we tested whether the exercise-responsiveness of Cytor is also conserved in these species. Indeed, Cytor mRNA levels were found to be increased in rat vastus lateralis (FIG. 2A) and soleus (FIG. 2B) after exercise, but not in the left ventricle of the heart (FIG. 2C), suggesting a skeletal muscle-specific induction of Cytor upon exercise. Moreover, inbreeding rats for high vs low aerobic exercise capacity for 34 generations selectively enriched CytormRNA in skeletal muscle of high capacity runners (HCR) compared to low capacity runner (LCR), while no difference was detected in the left ventricle (FIG. 2D).

We next assessed the responsiveness of Cytor to exercise in mice. Following exercise, Cytor levels were increased in mouse skeletal muscle independently of genetic background (C57BL/6JRj or DBA/2J) (FIG. 3A). Increased post-exercise Cytor levels most likely originated from muscle cells as Cytor was found to be elevated also in cultured mouse skeletal muscle cells (myotubes) after EPS (FIG. 3B). The BXD cohort is a recombinant inbred mouse population with substantial genetic heterogeneity, presenting a useful resource to study correlations between phenotypes and tissue gene expression. Skeletal muscle of different BXD mouse strains showed considerable variation in Cytor expression, which was not affected by diet. Interestingly, Cytor expression in the BXD cohort correlated positively and robustly with various exercise phenotypes in chow and high fat diet fed mice. Taken together, these results demonstrate that exercise increases the expression of the lncRNA Cytor with potentially positive functions in skeletal muscle.

### Example 2: Cytor Promotes Myogenic Differentiation

To investigate the role of Cytor RNA in skeletal muscle physiology, we knocked down Cytor using the potent antisense oligonucleotide (Gapmer), Gap05a, in C2C12 myotubes (FIG. 4A) and performed RNA sequencing. Transcriptome analysis revealed that Cytor knockdown led to reduced expression of genes encoding structural proteins of skeletal muscle (FIG. 4B). Expression of genes flanking Cytor remained unaltered, indicating that Cytor does not regulate its neighbouring genes in skeletal muscle (Bcl2l11 and Anapc1; FIG. 4C).

To explore the global effects of Cytoron skeletal muscle transcriptome, we conducted gene set enrichment analysis of the transcriptome data. Myogenesis (M5909) was the most downregulated pathway in Cytor diminished myotubes. Consistently, the myogenesis pathway (M5909) was also among the strongest correlated pathways with CYTOR in human skeletal muscle biopsies from Genotype Tissue Expression Project (GTEx) database. In line with this finding, Cytor/CYTOR expression increased during mouse and human myoblast differentiation (FIG. 5A-B). Surprisingly, type II myosin isoforms (Myh4, My/i) were more affected by Cytor knockdown than genes encoding slow twitch type I fibers (Myh7, My/3). Similarly, CYTOR mRNA levels in human skeletal muscle correlated significantly with genes encoding type II, but not with type I muscle fibers. In extensor digitorum longus (EDL), consisting predominantly of type II muscle fibers, Cytor displayed also higher expression compared to soleus, composed mostly of type I muscle fibers (GSE112716) (FIG. 6A), raising the hypothesis that Cytor might be involved in regulation of muscle fiber types.

To investigate the possibility that Cytor might affect myoblast differentiation, we altered its expression in C2C12 cells and evaluated cell morphology and gene expression patterns. Knockdown of Cytor in C2C12 myotubes decreased myotube area, reduced the number of nuclei present per myotube (FIG. 6B), and myotube diameter (FIG. 6C). Cytor knockdown in C2C12 myoblasts also reduced the differentiation ability of myoblasts (FIG. 7A-C). Progress in CRISPR-Cas9 technology has provided the opportunity to endogenously upregulate gene expression by epigenetic alterations without cutting DNA (sp-CRISPRa). We harnessed this nuclease-deficient Cas9 technology from Streptococcus pyogenes (spdCas9) to create a C2C12 cell line stably expressing spdCas9 together with the transcriptional activators VP64, MS2, P65 and HSF1. To endogenously overexpress Cytor we additionally introduced the single best performing guide RNA (gRNA) or a combination of gRNAs (gRNApool) targeting the promoter of Cytor. sp-CRISPRa-mediated overexpression of Cytor in C2C12 myoblasts reduced myoblast proliferation, and enhanced myotube differentiation manifested by increased myotube area (FIG. 7D left), elevated number of nuclei present per myotube (FIG. 7D right), and thicker myotube diameter (FIG. 7E) in Cytor overexpressing cells compared to controls. These results were consistent with different genetic constructs either silencing (Gapmers Gap05a, Gap05e) or overexpressing (guide RNAs gRNA4, gRNApool) Cytor, implying that the observed effects were unlikely due to off-target events (FIGS. 6B and 7D). In addition, gene expression profiling revealed Cytor to regulate expression of type II myosin isoforms (FIG. 8A). This regulation was confirmed by phosphofructokinase activity (FIG. 8B), which is more prevalent in type II muscle fibers. While Cytor was indispensable for type II myotube maintenance (FIG. 6B and FIG. 8A) and specification (FIG. 7A-C), Cytor overexpression directed myogenic differentiation towards type II myosin isoforms, thereby underscoring its role in deciding myoblast differentiation fate. Overall, our findings in vitro implicate a significant role for Cytor in promoting myogenesis and suggest a myoblast differentiation bias towards type II muscle fibers.

### Example 3: Bidirectional Cytor Gene Manipulation Alters Muscle Morphology and Function In Vivo

Deterioration of type II muscle fibers in size and number is a hallmark of ageing. Therefore, we examined the hypothesis that Cytor is involved in pathophysiogical muscle alterations inflicted by ageing. Indeed, Cytor expression was reduced upon ageing in mouse skeletal muscle, which was accompanied by reduced expression of the type II myosin heavy chain genes Myhc-IIa (Myh2) and Myhc-IIb (Myh4), but not the type I Myhc-I (Myh7) (FIG. 9A), particularly in the predominant type II muscles Gastrocnemius and EDL (FIG. 9B-D). Ageing-induced reduction of type II, but not type I fibers, was confirmed in humans in a publicly available microarray data set (GSE25941) (FIG. 10A) and has previously been reported in humans and rodents.

We next evaluated the effect of bidirectional Cytor gene manipulation on gastrocnemius muscle function and morphology in young and old mice. While Gapmer injections in gastrocnemius muscles of both hindlimbs were used to knockdown Cytor in young mice, the large size of Streptococcus pyogenes derived dCas9 (spdCas9), which was used in cells, presents a major challenge to activate endogenous gene expression in vivo as it exceeds the genome-packaging capacity of AAVs. To overcome this limitation, we switched to the catalytically inactive Cas9 protein derived from Staphylococcus aureus (sadCas9) fused to the transcriptional activator VP64 and a gRNA (sa-CRISPRa), which we were able to package into AAV9. Hence, to overexpress Cytor in aged mice, AAV9 carrying sa-CRISPRa including a gRNA, which we tested in C2C12 cells beforehand was injected intramuscularly in both gastrocnemius muscles of 18-months-old mice.

Cytor knockdown in gastrocnemius of young mice led to approximately 50% reduction in Cytor expression (FIG. 10B), resulting in a reduction of gastrocnemius muscle mass by 29% (FIG. 10C) without changing body weight (FIG. 10D). Functionally, knockdown of Cytor in young mice impaired hindlimb grip strength (FIG. 11A), and uphill running performance (FIG. 11B). Morphologically, Cytor knockdown in young mice reduced muscle fiber diameter (FIG. 11C), increased number of centralized nuclei (FIG. 11D), and reduced the proportion of type II myofibers (FIG. 12A and FIG. 12B). These findings were accompanied by increased inflammation and fibrosis (FIG. 12C). Our findings indicate that Cytor is indispensable for normal muscle maintenance, and young muscles lacking normal Cytor expression display features of ageing muscle, such as reduction of type II muscle fiber proportion.

Importantly, intramuscular injection of AAV9-sadCas9-VP64-U6-gRNA3 (sa-CRISPRa) in gastrocnemius muscle recovered the age-related reduction in Cytor expression at the age of 24 mo, and rescued ageing-associated loss of muscle mass (FIG. 10C) without changing body weight (FIG. 10D). Moreover, Cytor overexpression in gastrocnemius muscles ameliorated the ageing-induced decline in hindlimb grip strength (FIG. 11A) and uphill running performance (FIG. 11B). Gastrocnemius muscle fiber diameter in aged mice increased upon Cytor overexpression (FIG. 11C), possibly contributing to the increased muscle mass (FIG. 10C). Ageing-induced centralized nuclei only showed a trend for reduction (FIG. 11D) after Cytor overexpression. These improvements came along with a recovery of the age-associated decline in the proportion of type II muscle fibers (FIG. 12A), which was further confirmed by mRNA expression of specific muscle isoforms (FIG. 12D) and phosphofructokinase activity (FIG. 12B).

In summary, ageing attenuates Cytor levels in skeletal muscle and in vivo bidirectional gene manipulation of Cytor reveals that Cytor knockdown in skeletal muscle recapitulates functional and morphological hallmarks of a sarcopenic muscle, whereas recovering Cytor expression in aged muscle ameliorates the ageing-inflicted impairments in muscle mass and morphology.

### Example 4: A Skeletal Muscle Cis-eQTL Regulates CYTOR Expression

Next, we investigated the effect of genetic diversity on human CYTOR expression. To this end, we discovered 9 haploblocks within CYTOR (±50 kb), which contained several CYTOR cis-expression quantitative trait loci (cis-eQTLs) in human skeletal muscle biopsies from the GTEx database. The most significant cis-eQTLs were rs74924495, rs72624662 and rs74360724, located within a 12 kb region from each other. These SNPs were in high linkage disequilibrium with each other (r2>0.8) with minor allele frequencies 42-44%.

We next attempted to pinpoint the causal variant of these 3 SNPs representing the strongest cis-eQTLs. To delineate the causal variant of CYTOR expression from these 3 SNPs in human skeletal muscle, we performed several functional experiments.

First, we cloned each allele of the 3 SNPs (G and A for each SNP) into a luciferase plasmid, with total 7 plasmid constructs cloned (including one control plasmid without any insertion; EV). In human myoblasts, the 2 plasmids harboring the G and A alleles of rs74360724 displayed increased luciferase activity relative to plasmids harbouring rs74924495 and rs72624662 alleles (FIG. 13A), suggesting that the region around rs74360724 contains a CYTOR boosting cis-regulatory element. In addition, the minor A allele of rs74360724 increased luciferase expression more than the major G allele (FIG. 13A), which is in line with the dose-dependent effect of the rs74360724 alleles (GG, GA, AA) on CYTOR expression in human skeletal muscle in GTEx.

Second, using the recently optimized CRISPRi technology to induce heterochromatinization at promoters, we employed 3 gRNAs, each targeting the genomic region of the 3 SNPs, for epigenetic silencing of the putative regulatory elements using nuclease-deficient Cas9 (dCas9) fused to KRAB chromatin repressor domain and MeCP2 repressor protein (dCas9-KRAB-MeCP2). Introduction of dCas9-KRAB-MeCP2 into human myoblasts to silence the chromatin regions encompassing rs74924495, rs72624662 or rs74360724, increased H3K9me3 occupancy at these regions to a similar degree (FIG. 13B). However, only silencing rs74360724 resulted in reduced CYTOR expression despite being >80 kb away from the promoter (FIG. 13C), suggesting the presence of a putative CYTOR enhancer element. Enhancer elements are characterized by the presence of H3K27ac and H3K4me1 histone modifications. In human primary myoblasts, we found indeed an enrichment of these two histone modifications in the genomic region surrounding rs74360724. Furthermore, CRISPR/Cas9 mediated deletion of this enhancer element in human primary myoblasts reduced expression of CYTOR (FIG. 13D).

Third, to evaluate a physical interaction between the promoter of CYTOR and the cis-regulatory element harbouring rs74360724, the promoter of CYTOR was immunoprecipitated and tested for co-immunoprecipitation of this putative enhancer element. To this end, dCas9 fused to an HA-tag together with a non-targeting empty gRNA (EV), or a gRNA targeting the promoter of CYTOR was introduced into HEK293 cells. This strategy has been shown to identify enhancer-promoter interactions. To test whether our approach to tag the CYTOR promoter with HA was successful, we performed qPCR of the CYTOR promoter after chromatin immunoprecipitation of the HA tag. While neither the EV, nor IgG amplified the qPCR signal of the promoter of CYTOR, the HA-precipitated samples amplified the qPCR signal of the CYTOR promoter (P), validating that the HA-tag successfully labelled the promoter of CYTOR. Importantly, the putative enhancer (E) element harbouring rs74360724, but not the regions of rs74924495 or rs72624662, was pulled down together with the HA-tagged promoter of CYTOR, demonstrated by qPCR amplification of the putative CYTOR enhancer. Looping of the region adjacent to rs7436074 to the promoter of CYTOR was confirmed by the public available “Genehancer” tool. Collectively, these results implicate a cis-regulatory enhancer element in the regulation of CYTOR expression with carriers of rs74360724 (G->A) displaying elevated CYTOR levels in human skeletal muscle.

We next evaluated how electrical pulse stimulation (EPS), mimicking exercise in human myotubes, modulates CYTOR expression by measuring histone acetylation of the CYTOR promoter and the newly discovered enhancer region. Following EPS, induction of CYTOR mRNA was observed again, which coincided with H3K27 acetylation (H3K27ac) of the CYTOR promoter and the rs74360724 enhancer region, but not with the regions adjacent to rs74924495 or rs72624662, suggesting that activation of both promoter and enhancer might contribute to the increase in CYTOR expression upon exercise.

### Example 5: Elevated CYTOR Levels Enhance Fast-Twitch Myogenesis and Fitness in Ageing

To study whether rs74630724 affects CYTOR expression in a cell-autonomous manner, we measured CYTOR expression in human myoblasts from 21 different donors including young and old individuals, whom we genotyped for rs74630724. Similar to muscle tissue, the A allele of rs74360724 was associated with increased CYTOR expression in primary myoblasts (FIG. 14A), demonstrating a cell-autonomous effect of genetic regulation on CYTOR expression. Donors homozygous for the AA-allele also showed a trend for reduced H3K27me3 occupancy compared to GG donors (FIG. 14B). Notably, CYTOR expression in myoblasts from aged donors was reduced compared to young donors (FIG. 14A), confirming our findings in mice (FIG. 9A), and suggesting a conserved muscular dysregulation of CYTOR expression across human and mouse ageing.

Next, we hypothesized that individuals having the CYTOR-increasing A-allele of rs74360724 might be protected against the age-dependent decline in fitness. To this end, we employed the Helsinki Birth Cohort Study (HBCS), a well-characterized Finnish population cohort (n=2003) composed of aged (70.92±2.68 yo) individuals, to study the effects of CYTOR cis-eQTL rs74360724 on performance in 6-min walk test, which was shown to identify sarcopenic patients with limited mobility. The CYTOR expression boosting A allele (FIG. 14A) was associated with increased distance walked during 6 minutes (FIG. 14C), consistent with Cytor overexpression induced improvements in aged mice (FIGS. 11A and B). These findings indicate that elevated CYTOR levels translate into improved age-related fitness.

To experimentally investigate the direct genetic effect of CYTOR on human myoblast differentiation, a biological process potentially able to compensate for the loss of myofibers in age-related sarcopenia, we evaluated aged human myotube morphology upon CRISPR-dCas9 mediated genetic upregulation of CYTOR. Using spdCas9-VP64 together with gRNAs targeting the human CYTOR promoter we were able to successfully introduce sp-CRISPRa into human myoblasts and increase CYTOR expression endogenously. In human myoblasts derived from an aged (76y) donor, heterozygous for rs74360724, CYTOR overexpression reduced primary myoblast proliferation, and instead enhanced myoblast differentiation, evidenced by increased myotube area (FIG. 15A), improved myofusion index, and elevated markers of the type II myosin isoforms, MYL1 and MYH2, but not of the type I marker MYL3 (FIG. 15B). Our results were similar for two different gRNAs (FIG. 15A-B), strongly minimizing the possibility of off-target effects. Thus, artificial elevation of CYTOR levels in aged human myoblasts improved muscle morphology by enhancing myoblast differentiation into type II myotubes. Taken together, these data suggest that genetic manipulation of CYTOR could have therapeutic potential to promote type II muscle fibers in muscle diseases, such as sarcopenia.

### Example 6: CYTOR Rejuvenates Muscle Morphology and Function in Aged C. elegans

Finally, given the cross-species conservation of myogenesis, and to study the potential benefits of human CYTOR on systemic muscle function in an in vivo ageing model, human CYTOR DNA was cloned into C. elegans under control of the muscle-specific promoter myo3p. This strategy was chosen because C. elegans do not express CYTOR orthologues natively. Worms harbouring human CYTOR (CYTOR +(myo3p)) showed similar muscle morphology compared to their N2 wild-type littermates (CYTOR − (wt)) at young age (day 1). However, at old age (day 15), unlike their N2 controls, CYTOR expressing worms displayed better aligned, and less deteriorated muscle fibers, resembling skeletal muscle morphology of young worms. These morphological improvements translated into functional gains in movement at old age (FIG. 16A), as well as higher maximum (FIG. 16B) and minimum (FIG. 16C) speed when challenged to move. In addition, CYTOR expressing worms had increased mobility (FIG. 16D), reduced paralysis (FIG. 16E), and increased survival (FIG. 16F) at old age. Collectively, these data show that skeletal muscle-specific expression of human CYTOR in C. elegans rejuvenates muscle morphology and function at old age, thereby strengthening the notion that CYTOR is a novel key player in healthy muscle ageing and demonstrating the potency of human CYTOR.

### Example 7: Cytor Modifies Chromatin Accessibility at Tead1 Binding Sites

In order to identify potential mediators of Cytor's effect on myoblast differentiation and fiber type bias, we generated a C2C12 cell line constitutively overexpressing endogenous Cytor (Cytor-OE) (FIG. 17A). In this stable C2C12 cell line, Cytor overexpression again promoted myoblast differentiation. LncRNAs have been shown to exert effects on transcription regulation by chromatin alteration; this is in particular observed for lncRNAs retained in the nucleus. To investigate this possibility, we performed Assay for Transposase-Accessible Chromatin using sequencing (ATAC-seq) to compare chromatin accessibility in control versus Cytor-OE myoblasts. As expected, transposase digestion fragmented DNA and mapped peaks were distributed across the genome with most peaks localizing within distal intergenic regions (30.31%), promoters (23.31%), or other intronic regions (21.44%). The first principal component, explaining 46% of the variance, separated control from Cytor-OE myoblasts, suggesting that overexpression of Cytor indeed altered chromatin accessibility. Gene ontology of differentially accessible genes showed cell differentiation as one of the most enriched pathways upon Cytor-OE, which is in line with our mouse and human in vitro results. Indeed, among the 1559 differentially accessible genes, promoter accessibility of the structural muscle genes Utrn and Tnc was increased upon Cytor-OE. Of note, Tnc deficient mice have previously been shown to present selective atrophy of type II muscle fibers.

Performing de novo Hypergeometric Optimization of Motif EnRichment (HOMER) analysis on genomic areas which either lost or gained accessibility revealed several transcription factors with altered chromatin accessibility upon Cytor-OE. The Mef2 transcription factor, whose accessibility was reduced upon Cytor-OE, has previously been implicated in promoting type I muscle fibers. Reduced chromatin accessibility at Tead1 bindings sites upon Cytor overexpression also caught our attention, as Tead1 silencing has been shown to specifically promote type II muscle fibers in vitro and in vivo. Conversely, muscle-specific overexpression of Tead1 has been shown to reduce type II fibers. In line with the HOMER analysis, several direct Tead1 binding targets reported in C2C12 cells (Tef, Tcta, S/c29a1, Ndufa6) indeed lost chromatin accessibility upon Cytor-OE (FIG. 17B, left). Consistent with this, Tead1 occupancy at the promoters of these target genes was reduced (FIG. 17B, middle) and mRNA expression was reduced upon Cytor overexpression (FIG. 17B, right).

We reasoned that the differentiation-promoting effect of Cytor might be dependent on Tead1 and therefore co-overexpressed Tead1 in C2C12 cells stably overexpressing Cytor (FIG. 17A). Compared to Cytor-OE alone, overexpressing Tead1 together with Cytor (FIG. 18A) reduced myotube area (FIG. 18B), myofusion (FIG. 18C) and abolished expression type II myosin isoforms (FIG. 18D). No effect was observed on the expression of the type I myosin isoform (FIG. 18D). These data are in line with previous reports showing decreased type II fiber percentage upon Tead1 overexpression and suggest that Cytor's effect on myoblast differentiation into type II myotubes is dependent on excluding Tead1 binding to chromatin. In line with this notion, we observed that disruption of Tead1 function genetically with a specific siRNA targeting Tead1 (FIG. 18A) or pharmacologically using Verteporfin, a Tead1 inhibitor, was sufficient to increase myotube area (FIG. 18B), myofusion (FIG. 18C) and induce the expression of type II, but not type I, myosin isoforms (FIG. 18D), similarly to Cytor overexpression alone. These findings point towards Tead1 mediating at least part of Cytor's effect on myoblast differentiation into type II myotubes.

To assess whether Tead1 silencing was able to recapitulate the beneficial effect of Cytor in C. elegans in vivo, we silenced the Tead1 orthologue egl-44 or treated the worms with verteporfin and found that either strategy phenocopied the beneficial effect of Cytor in aged C. elegans both functionally and morphologically (FIG. 19A-F).

### Materials and Methods

Study design. The objective of this study was to identify lncRNAs altered by exercise in human skeletal muscle. Unbiased RNA profiling indicated the myogenesis pathway to be regulated by Cytor and all assays to measure myogenesis were reproduced in multiple animals. All animals used in the experiments were randomly assigned to experimental or control groups. Animals that showed signs of severity, predefined by the animal authorizations, were euthanized. These animals, together with those who died spontaneously during the experiments, were excluded from the calculations. These criteria were established before starting the experiments. All mouse phenotyping was performed blinded. Calculation of sample sizes for both cell and animal experiments were determined based on previous findings. For motility, fitness and death scoring experiments in C. elegans, sample size was estimated based on the known variability of the assay. Sample sizes, replicates, and statistical methods are specified in the figure legends.

Statistical analyses. Differences between two groups were assessed using two-tailed t tests. Differences between more than two groups were assessed by using one-way or two-way analysis of variance (ANOVA), unless stated otherwise. GraphPad Prism 6 (Prism) was used for statistical analyses. Variability in plots and graphs is presented as standard error mean (SEM). All p<0.05 were considered to be significant. *p<0.05; **p≤0.01; ***p≤0.001; ****p≤0.0001. Mouse experiments were performed once.

Rat studies. Female Sprague-Dawley rats (200-220 g) were used for the exercise study. Rats were exercised using an incremental protocol at 25 degrees incline starting at 6 m/min and increasing speed by 0.03 m/s every 2 min until exhaustion. Animals were sacrificed at 0 h, 1 h, 3 h, 6 h, and 24 h after exercise and vastus lateralis, soleus and left ventricle were excised and immediately snap frozen in liquid nitrogen. Another cohort of rats were inbred for low- or high running capacity for 34 generations. Soleus and left ventricle from these rats were excised and immediately snap frozen in liquid nitrogen for subsequent gene expression analyses. All rats were acclimatized to animal facilities for seven days prior to the experiments. Four animals were housed per cage (22° C.) with free access to standard chow and water. All experiments were approved by the Norwegian Animal Research Authority before the start of the project (FOTS #5829).

Mouse studies. Mouse chronic exercising was performed three times a week for 2 or 4 weeks before mice were sacrificed and gastrocnemius muscles were dissected and snapfrozen for biochemical analyses. Young (3-month-old) and aged (18-month-old) male C57Bl/6JRj mice were purchased from Janvier Labs. Body weight was measured before and after treatment. 3 mo mice were caged 4 animals/cage and bilaterally injected intramuscularly with 1.5 μg/g Gapmers (Exiqon, Qiagen) diluted in PBS at 0.8 ug/uL in gastrocnemius muscle 2 times a week for 6 weeks. 18 mo mice were single caged and bilaterally injected intramuscularly once with 1*10E¹¹ viral genomes (VG) in gastrocnemius. One week after phenotyping, left gastrocnemius muscles of 5 mo and 24 mo mice were rapidly removed, weighed and snap-frozen in liquid nitrogen while right gastrocnemius muscles were embedded for histological analyses. All animals were housed in micro-isolator cages in a room illuminated from 7:00 AM to 7:00 PM with ad libitum access to chow diet and water. All animal experiments were approved by the local authorities (Swiss Veterinary Office; 2890.1C).

Endurance running performance mice. The exercise regimen on a treadmill commenced at a speed of 15 cm/s. Every 12 minutes, speed was increased by 3 cm/s. To engage hindlimb usage an inclination of 10 degrees was used. Mice were considered exhausted and removed from the treadmill if 5 or more shocks (0.1 mA) per minute were received for two consecutive minutes. The distance travelled was registered as maximal running distance.

Grip strength. Muscle strength was assessed by grip strength test. The grip strength of each mouse was measured on a pulldown grid assembly connected to a grip strength meter (Columbus Instruments). The mouse was drawn along a straight line parallel to the grip, providing peak force. Hindlimb grip strength was calculated by subtracting forelimb grip strength from four limb grip strength. The experiment was repeated three times, and the highest value was included in the analysis. Experiments were repeated twice.

BXD mouse population. 34 BXD strains from the mouse genetic reference population were housed in micro-isolator cages in a room illuminated from 7:00 AM to 7:00 PM with ad libitum access to either high fat or chow diet and water. Upon sacrifice gastrocnemius muscles were excised, snap frozen, and pooled (3-5 mice per strain) for subsequent analysis of Cytor expression by RT-qPCR. For the in silico correlation of Cytor expression levels with exercise phenotypes including maximal oxygen uptake in an untrained state (VO2max), oxygen kinetics in trained and untrained state, maximal running distance, and gastrocnemius muscle mass we used publicly available data from the BXD downloaded from GeneNetwork.org. Spearman's r was used to establish correlations between phenotypes and Cytor mRNA levels in gastrocnemius muscle. All BXD experiments were authorized by animal license 2257.1 in Canton of Vaud, Switzerland. The chorogram was obtained using the R/chorogram package.

Transgenic Caenorhabditis Elegans. C. elegans strains were cultured at 20° C. on nematode growth medium (NGM) agar plates seeded with E. coli strain OP50 unless stated otherwise. Strains used in this study were the wild-type Bristol N2 and COP2054. N2 strain was provided by the Caenorhabditis Genetics Center (University of Minnesota). For RNAi experiments, worms were exposed to egl-44 RNAi or an empty vector control plasmid using maternal treatment to ensure robust knock down of the investigated gene. Verteporfin was dissolved in DMSO, and used at a final concentration of 10 uM. Worms were exposed to verteporfin starting from L4 stage. Verteporfin or DMSO was added to agar before preparing plates. To ensure a permanent exposure to the compound, plates were changed twice a week. The COP2054 worm strain expressing human CYTOR was generated by a custom transgenic service (Knudra Transgenics, Salt Lake City, UT). Briefly, Mosi-mediated Single Copy Insertion (MosSCI) started with the construction of myo3p::CYTOR::tbb2u, where the myo3p promoter and tbb-2 3′-UTR were taken from the N2 genome, and CYTOR was synthesized with codon and intron optimization for C. elegans. This construct was cloned into the pNU936 backbone for delivery to the ttTi5605 locus on chromosome II. The pNU936 backbone containing myo3p::CYTOR::tbb2u including an unc-119 rescue cassette, was injected into the COP93 [unc-119(ed3); ttTi5605] strain. The MosSCI plasmid was injected at 15 ng/mL. Injected animals were screened for unc-119 rescue. Candidates absent of array markers were homozygous. This was confirmed by PCR for amplicons specific to targeted insertion and identified as COP2054:knuSi831 [pnu2144(myo3p::CYTOR::tbb-2u,unc-119(+))]II;unc-119(ed3)III.

C. Elegans motility. C. elegans movement analysis was performed at day 15 of adulthood, using the Movement Tracker software as done previously. The experiments were repeated at least twice.

C. Elegans paralysis and death score. 45 to 60 worms per condition were manually scored for paralysis after poking. Worms that were unable to respond to any repeated stimulation were scored as dead. Results are representative of data obtained from at least two independent experiments.

C. Elegans morphology. A population of ˜100 worms was washed in M9 and frozen in liquid nitrogen. Immediately after, worms were lyophilized using a centrifugal evaporator and permeabilized using ice cold acetone. 2 U phalloidin (Thermo Scientific) was resuspended in 20 μl of a buffer containing: Na-phosphate pH 7.5 (final concentration 0.2 mM), MgCl₂ (final concentration 1 mM), SDS (final concentration 0.004%) and dH₂O to volume. The worms were incubated for 1 h in the dark and then washed twice in PBS, prior to mounting on 2% agar pad. Confocal images were acquired with Zeiss LSM 700 Upright confocal microscope (Carl Zeiss AG) under non-saturating exposure conditions. Image processing was performed with the Fiji software.

Skeletal muscle gene expression in Genotype-Tissue Expression (GTEx) project. For RNA gene expression analyses, we employed post-mortem skeletal muscle biopsies from the GTEx gene expression (dbGAP, approved request #10143-AgingX). As measures of gene expression, we used residual expression levels of transcripts adjusting for the published GTEx covariates. For enrichment analysis or correlation analyses of CYTOR in human skeletal muscle in GTEx genes were ranked based on their Pearson correlation coefficients with expression CYTOR, and GSEA was performed to find the enriched gene sets co-expressed with CYTOR by using R/fgsea package.

Expression quantitative trait loci (cis-eQTL) analyses. For eQTL analyses, we used the GTEx v8 genotypes obtained from the GTEx web portal (https://gtexportal.org/). For identification of CYTOR cis-eQTLs in skeletal muscle, SNPs±50 kb from the CYTOR start and stop side were included from the 1000 Genomes Project and HBCS and tested for their effect on CYTOR expression in skeletal muscle (GTEx). SNPs with minor allele frequency >10% were included, and SNPs with r2>0.2 were incorporated in the same haploblock based on linkage disequilibrium information from the version 5 of the 1000 Genomes Project. As there were 9 haploblocks within CYTOR we used Bonferroni correction of P<0.0055 for identification of significant cis-eQTLs.

Helsinki Birth Cohort Study (HBCS). The HBCS includes 13,345 individuals born in Helsinki between 1934 and 1944. The clinical study protocol was approved by the Ethics Committee of Epidemiology and Public Health of the Hospital District of Helsinki and Uusimaa. Written informed consent was obtained from each participant before any study procedure was initiated. The result of the 6-min walk test was expressed as age (for each 5-year group) and sex-standardized percentile scores. DNA was extracted from blood samples and genotyping was performed with the modified Illumina 610k chip by the Wellcome Trust Sanger Institute, Cambridge, UK, according to standard protocols. Genomic coverage was extended by imputation using the 1000 Genomes Phase I integrated variant set (v3/April 2012; NCBI build 37/hg19) as the reference sample and IMPUTE2 software. Before imputation the following QC filters were applied: SNP clustering probability for each genotype >95%, Call rate >95% individuals and markers (99% for markers with MAF <5%), MAF >1%, HWE P>1×10⁻⁶. Moreover, heterozygosity, gender check and relatedness checks were performed, and any discrepancies removed. We performed linear regressions with SNPtest assuming an additive genetic model. We adjusted for age, sex, highest education achieved (basic or less/upper secondary/lower tertiary/upper tertiary) and smoking (yes/no).

Genotyping human myoblasts. 21 human myoblasts were cultured in 6 well plates in triplicates. DNA was isolated using the kit NucleoSpin Tissue (Macherey Nagel) according to the manufacturer's protocol. PCR for rs74360724 was performed using the KAPA2G mix (Km5104, Kapa Bioscience) according to the manufacturer's protocol. DNA was cleaned up using the PCR cleanup gel extraction kit (Macherey Nagel) according to the manufacturer's protocol prior to sequencing (Microsynth) with the reverse primer.

Adeno associated virus (AAV) production. Recombinant serotype 9 adeno-associated viral (AAV) vectors were produced according to standard procedures. In brief, HEK AAV-293 cells (Agilent) were co-transfected with the pAAV (AAV-CMVsadCas9-VP64-U6-gRNA or AAV-CMV-sadCas9-VP64-U6-gRNA3) and pDP9 plasmids. The AAV9 particles contained in the cell lysates were isolated on an iodixanol gradient followed by ion exchange FPLC using a HiTrap Q-FF column (5 ml, GE Healthcare) connected to an AKTA start chromatography system (GE Healthcare). After buffer exchange (resuspension in DPBS) and concentration on a centrifugal filter (cut-off 100 kDa, Amicon Ultra, Millipore), the vector suspensions were titered by real-time PCR for the presence of genome-containing particles (VG). AAV9 intramuscular injections in mice were done in both gastrocnemius muscles with 1 E11 VG for each muscle, at three different injections sites.

Immunohistochemistry. Right Gastrocnemius muscles were embedded in Thermo Scientific™ Shandon™ Cryomatrix™ and frozen in isopentane, cooled in liquid nitrogen, for 1 min before being transferred to dry ice and stored at −80° C. 8 μm cryosections were cut from fresh frozen samples, dried for 10 min, and placed in Harris Hematoxyline (Gill II Pap 1; Biosystems 3873.2500) for 5 min nuclear staining. Sections were then washed with H₂O, differentiated in 1% acid-alcohol (700 mL 100% EtOH (VWR 20820.362), 10 mL Hydrochloric acid 37% (Sigma 30721) and 290 mL H₂O) for a few seconds, washed again with continuous flow of H2O for 10 min, and incubated in Eosine-Phloxine working solution consisting of 1% Eosine Y (Sigma E4382; diluted in H₂O), 1% Phloxine B (Sigma P2759), 95% EtOH and Glacial acetic acid for 10 min to stain the cytoplasm. After dehydration and clearing the section were mounted (Eukitt, Merck 03989). Detection of inflammation was performed manually using a rat anti CD45 (clone 30F-11, Thermo fisher, #14-0451-82, diluted 1:200) 8 um fresh frozen sections. Tissues were fixed with methanol at −20° C. for 10 minutes and completely dried before peroxidase quenching and blocking. Primary antibody was incubated overnight at 4° C. After incubation of a rabbit Immpress HRP (Ready to use, Vector Laboratories), revelation was performed with DAB (3,3′-Diaminobenzidine, Sigma-Aldrich). Selected slides were stained with Sirius red F3B (C135782, Direct red 80) to assess collagen content (or fibrosis). Microscopy images of muscle fibers were imaged using VS120-SL slides scanner (Olympus). Six biological samples per group were analyzed. The fraction of centralized nuclei and measurement of the minimal Feret diameter in gastrocnemius muscles were determined using the ImageJ software quantification of laminin. The minimal Feret diameter is defined as the minimum distance between two parallel tangents at opposing borders of the muscle fiber. This measure has been found to be resistant to deviations away from the optimal cross sectioning profile during the sectioning process. Inflammation representing the regenerative stage of muscle was quantified by ImageJ software as proportion of inflammatory area over total area of the Gastrocnemius muscle cross section.

Immunofluorescence. Tissue immunostaining was performed on fresh frozen section of mice right gastrocnemius muscles. 8 μm sections were cut from OCT-embedded samples. Sections were dried for 10 min and rehydrated with PBS 3×5 min and blocked with 1% BSA (in PBS) for 30 min. Primary antibody incubation was performed under gentle agitation overnight at 4° C. and subsequently incubated with secondary antibody 1 h at RT after washing 2×5 min with PBS. Then sections were mounted (Fluoromount G, SouthernBiotech) and imaged VS120-SL slides scanner (Olympus). Antibodies to stain for different muscle isoforms were: Myh7 (BA-F8, DHSB), Myh2 (Sc-71, DHSB), Myh4 (BF-F3, DHSB) and laminin (L9393, Sigma). Secondary antibodies used were: Goat anti mouse IgG2b Alexa 350 (#A-21140, Thermo Fisher), Goat anti mouse IgG1 Alexa 594 (#A-21125, Thermo Fisher), Goat anti mouse IgM Alexa 647 (#A-21238, Thermo Fisher) and donkey anti rabbit Alexa 488 (#21206, Thermo Fisher). Six biological samples were analyzed per group. Image processing was done using Fiji. For every image, the proportion of isoforms was calculated as the number of stained fibers for the same isoform divided by total fibers including non-stained fibers.

Cell culture. The C2C12 mouse myoblast cell line was obtained from the American Type Culture Collection (CRL-1772TM). C2C12 and HEK293T cells (CRL-3216™, ATCCR) were cultured in growth medium consisting of Dulbecco's modified Eagle's medium (Gibco, 41966-029), 20% Fetal Bovine Serum (Gibco, 10270-106) and 100 U/mL penicillin and 100 mg/mL streptomycin (Gibco, 15140-122). Differentiation of Cytor/CYTOR overexpressing cells or Tead1 silenced cells were assessed in culture medium. Human skeletal muscle myoblasts (Lonza, Switzerland) were cultured in DMEM/F12 Glutamax (31331093, Gibco) supplemented with 20% FBS (Gibco, 10270-106) and 1% penicillin/streptomycin (15140-122, Gibco). Human skeletal muscle myoblasts from young, old and DMD donors were obtained from the Groupement Hospitalier Est (CBC BioTec, Centre de Ressources Biologique) and cultured in Ham F10 medium (11550043, Thermo Fisher) with 12% FBS (10270-106, Gibco) and 1% penicillin/streptomycin (Gibco, 15140-122). Muscle cell differentiation was achieved by substituting FBS with 2% horse serum (Gibco, 16050-122). For detachment Trypsin-EDTA 0.05% (25300-062, Gibco) was used for all cell lines. All cells were maintained at 37° C. and 5% CO₂. Cell lines were continuously tested for mycoplasma contamination.

Cell transfection and transduction. Cell transfections were done using TransiT (Mirus) according to the manufacturer's protocol with a 3:1 ratio of transfection agent to DNA. 24 well plates were transfected with 150 ng DNA, 12 well plates with 300 ng DNA, and 6 well plates with 1 ug DNA. To transfect 25 nM GapmeR or siRNA in C2C12, LipofectamineR RNAiMAX (Thermo Scientific, 13778100) was used following manufacturer's instructions. Lentiviruses were produced by cotransfecting HEK293T cells with lenti plasmid (lentiSAMv2 or lentiSAMv2-gRNA4 mouse or lentiSAMv2-gRNA3 human or lentiSAMv2-gRNA4), the packaging plasmid psPAX2 (addgene #12260) and the envelope plasmid pMD2G (addgene #12259), in a ratio of 4:3:1, respectively. Transfection medium was removed 24 h after transfection and fresh medium was added to the plate. Cell supernatants were collected at 48 h and filtered through a 0.45-μm filter. Cells to be transduced were seeded 24 h prior to infection and then transduced with virus-containing supernatant supplemented with 8 μg/mL polybrene (Millipore). Cells were left to recover for 24 h in growth media before blasticidin (2.5 μg/mL) selection for 7d.

C2C12 subcellular fractions. To separate nuclear and cytosolic fractions of C2C12 mouse myoblasts, the phenol-free Protein and RNA Isolation System (PARIS) kit was used (AM1921, Invitrogen) according to the manufacturer's protocol. In brief, cells were washed with PBS and lysed on ice with the Cell Fractionation Buffer supplemented with beta mercapethanol. Cell were then centrifuged for 5 min, 500 g at 4° C. to separate nuclear and cytosolic fractions by collecting the supernatant and pellet, respectively. Ice-cold Cell Disruption Buffer was added to the nuclear pellet followed by vortexing for lysis. Lysis/Binding solution was added to the nuclear and cytosolic fraction and an equal amount of 100% EtOH prior to transferring the mixtures to a Filter Cartridge and centrifugation. The Filter Cartridges were then washed with the supplied wash buffers and RNA was eluted with preheated Elution Solution followed by DNase digestion.

Immunocytochemistry. C2C12 cells or human skeletal muscle myoblasts cultured on a sterilized cover slip in 6-well plates (Greiner bio-one, CELLSTAR, 657160) were fixed in Fixx solution (Thermo Scientific, 9990244) for 15 min and permeabilized in 0.1% Triton X-100 (Amresco, 0694) solution for 15 min at 20° C. Cells were blocked in 3% BSA for 1 h at 20° C. to avoid unspecific antibody binding and then incubated with primary antibody over night at 4° C. with gentle shaking. MyHC was stained using the MF20 primary antibody (1:200, Invitrogen, 14-6503-82) for C2C12 cells and in human muscle cells with a MYL2 antibody (1:140, Abcam, ab79935). The next day cells were incubated with secondary antibody (Thermo Fisher #A10037 for MF20 and #A-21206 for MYL2) for 1 h at 20° C. and nuclei were labelled with DAPI. Immunofluorescence images were acquired using bright field and confocal microscopy. Images were obtained analyzing z-stacks (max intensity) at multiple positions under non-saturating exposure conditions. Image processing was performed using the Fiji software. 10 images were processed for every biological sample and averaged. The myofusion index was calculated as the ratio of nuclei within myotubes to total nuclei. Myotube diameter was measured for 10 randomly chosen myotubes per image. Myotube area was calculated as the total area covered by myotubes.

RNA isolation and real time qPCR. RNA was isolated using the RNeasy Mini kit (Qiagen, 74106) with the DNase I digestion step and reverse transcribed with the High-Capacity-RNA-to-cDNA kit (Thermo Fisher, 4387406). Gene expression was measured by qPCR using the Power SybrGreen Master mix (Thermo Fisher, 4367659). All quantitative polymerase chain reaction (qPCR) results were calculated relative to the mean of the housekeeping gene Gapdh/GAPDH. The average of two technical replicates was used for each biological data point.

Electrical pulse stimulation. Human muscle cells (Lonza, Switzerland) or C2C12 mouse myoblasts were differentiated in a 6 well plate with 2% horse serum and connected to a C-dish electrode (IonOptix) to apply electrical current through a carbon electrode immersed in differentiation medium. EPS-mediated contraction of myotubes was verified using light microscopy. As previously optimized, 30V were used with a pulse duration of 2 ms at 1 Hz frequency for 30 min. Cells were harvested at 0, 30, 60, 120, 180 and 240 min after EPS.

CRISPR/Cas9 mediated genomic deletion. First, gRNA sequences flankingrs74360724 were cloned into lenti-CRISPRv2-puromycin (addgene #52961) and lenti-CRISPRv2-blasticidin (addgene #83480). Next, human primary myoblasts were double transfected (TransIT, Mirus) with lenti-CRISPRv2-puromycin (addgene #52961) and lenti-CRISPRv2-blasticidin (addgene #83480), containing a guide RNA targeting the genomic region 25 bp upstream and downstream, respectively of rs74360724. Next, cells were double selected with puromycin and blasticidin for 5 days and RNA was extracted (RNeasy Mini kit, 74106, Qiagen), cDNA was generated (Prime Script™ RT reagent kit with gDNA Eraser, RR047A, Takara Bio) and gene expression measured with real-time quantitative PCR (TB GreenR Premix Ex Taq™, RR420L, Takara Bio).

CRISPR dCas9 overexpression experiments. Five gRNAs for Cytor/CYTOR were designed with the help of the online GPP web portal tool (https://portals.broadinstitute.org/gpp/public/analysis-tools/sgrna-designcrisprai?mechanism=CRISPRa) using Streptococcus pyogenes PAM sequence (NGG) for sp-CRISPRa in vitro application or Streptococcus aureus PAM sequence (NNGRR) for sa-CRISPRa in vivo. The gRNAs with best predicted on- and off-target scores were selected for mouse and human. The oligonucleotides were synthesized and cloned into the gRNA-MS2 (#61424, Addgene) using Bbsl restriction enzyme, AAV-CMV-sadCas9-VP64-U6-gRNA using Bsal restriction enzyme and lentiSAMv2 (#75112, Addgene) using the BsmBI restriction enzyme (Genewiz, New Jersey). Insertion by cloning was verified by Sanger sequencing (Microsynth, Switzerland). To test the efficiency of the gRNAs for sp-CRISPRa in mouse cells, the cloned vectors with inserted gRNA sequences or the empty vector plasmid (gRNA-MS2) were transiently transfected (TransIT, Mirus) in C2C12 cells stably expressing spdCas9-VP64-MS2-P65-HSF1, and 48 h after transfection RNA was isolated, reverse transcribed and Cytor expression was measured by RT-qPCR. Cytor overexpression in mice was performed as shown previously for Lama1. To test the efficiency of the gRNAs for sa-CRISPRa, the cloned vectors with inserted gRNA sequences or the empty vector plasmid (AAV-CMV-sadCas9-VP64-U6-gRNA) were transiently transfected (TransIT, Mirus) in C2C12 cells, and 48 h after transfection RNA was isolated, reverse transcribed and Cytor expression was measured by RT-qPCR. To test the efficiency of the gRNAs for sp-CRISPRa in human cells, the cloned vectors with inserted gRNA sequences or the empty vector plasmid (lentiSAMv2) were transiently transfected (TransIT, Mirus) in human muscle cells (Lonza, Switzerland), and 48 h after transfection RNA was isolated, reverse transcribed and CYTOR expression was measured by RT-qPCR. Lentivirus was produced from lentiSAMv2, lentiSAMv2-gRNA3 and lentiSAMv2-gRNA4 to transduce aged human myoblast cell line 171. Lentivirus was also produced from lentiSAMv2 and lentiSAMv2-gRNA4 to transduce C2C12 mouse myoblasts and establish a cell line stably overexpressing Cytor.

Epigenetic silencing of cis-regulatory elements. First, gRNAs targeting rs74924495, rs72624662 or rs74360724 were designed using the UCSC CRISPR/Cas9 track. The oligonucleotides were synthesized and cloned into the phU6-gRNA (#53188, Addgene) using Bbsl restriction enzyme (Genewiz, New Jersey). Insertion by cloning was verified by Sanger sequencing (Microsynth, Switzerland). Next, human skeletal muscle myoblasts (Lonza, Switzerland) were cultured in 24 well plates and reverse co-transfected with a plasmid expressing dCas9-KRABMeCP2 (#110821, Addgene) together with either the empty phU6 plasmid or the subclones expressing the gRNAs using TransiT (Mirus). To achieve equimolarity these two plasmids were transfected at the ratio 2.4:1. Experiments were repeated at least twice.

Cell proliferation assay. Mouse C2C12 myoblasts or human primary myoblasts overexpressing Cytor/CYTOR were cultured in 96 well plates. The Brdu assay was performed as indicated in the manufacturer's protocol (Cell Proliferation ELISA, BrdU (colorimetric), 11 647 229 001, Roche). In brief, after incubation, MTT was added (0.5 mg/mL final concentration) and cells were incubated again for 4 h. Then Solubilization solution was added to the wells and after overnight incubation spectrophotometrical absorbance was measured with a 550 nm filter.

Phosphofructokinase activity. Phosphofructokinase activity in mouse gastrocnemius tissue and C2C12 myoblasts where Cytor was knocked down or overexpressed was measured according to the manufacturer's protocol (6-Phosphofructokinase Activity Assay Kit (Colorimetric), ab155898, Abcam). In brief, samples were homogenized with the supplied Assay Buffer and protein concentration was measured (Protein Assay Kit II, 5000002, Bio-Rad) to use 10 ug and 20 ug protein from tissue and cell samples, respectively for the assay. The reaction mix was incubated at 37° C. and absorbance at 450 nm was measured every 5 min.

CYTOR promoter tagging with dCas9-HA. The same gRNA sequence that upregulated CYTOR expression in human myoblasts was used (GTATGAAGAGAATGTCGGGAG, SEQ ID NO:38) as it was designed to target the promoter of CYTOR and cloned into the phU6-gRNA plasmid using Bbsl restriction enzyme (Genewiz, New Jersey). Insertion by cloning was verified by Sanger sequencing (Microsynth, Switzerland). HEK293 cells cultured in 15 cm dishes were co-transfected with dCas9-HA (#61355, Addgene) and the empty phU6 plasmid (EV) or its subclone expressing the gRNA sequence.

Chromatin immunoprecipitation followed by qPCR (ChIP-qPCR). After washing cells with PBS cells were crosslinked with 1% formaldehyde for 10 min at room temperature followed by quenching with glycine (final concentration of 0.125M) to stop the crosslinking reaction before harvesting cells with SDS Buffer (50 mM Tris-HCl (pH 8), 100 mM NaCl, 5 mM EDTA (pH 8.0), 0.2% NaN3, 0.5% SDS, 0.5 mM phenylmethylsulfonyl fluoride) complemented with protease inhibitor cocktail. Nuclei were pelleted by centrifugation for 6 min at 250 g and resuspended in ice-cold IP Buffer (IP buffer=1 volume SDS Buffer: 0.5 volume Triton Dilution Buffer (100 mM Tris-HCl, 100 mM NaCl, 5 mM EDTA, 0.2% NaN₃, 5% Triton X-100)) for sonication (Diagenode, Biorupter) to fragment lengths of 200-500 bp. Samples were divided for precipitation of IgG or specific antibody (HA, H3K27ac, H3K27me3, H3K9me3, Tead1) while 2.5% was used as input. Samples were incubated with 2.5 ug primary antibody overnight at 4° C. rotating and then incubated for 4 h at 4° C. with 50 uL washed beads (Magnetic Dynabeads A, Thermofisher #88836). After washing the beads with a wash buffer consisting of 1% Triton X-100, 0.1% SDS, 150 mM NaCl, 2 mM EDTA, 20 mM Tris-HCl cells were de-crosslinked using 120 ul of 1% SDS, 0.1M NaHCO₃ for 6 h at 65 C. Next, PB buffer was added and purification of DNA was done according to the manufacturer's protocol (MinElute PCR purification kit, Qiagen). 50 uL eluted DNA was used to perform RT-qPCR.

RNA sequencing library preparation and analyses. Total RNA from C2C12 cells was extracted by column using RNeasy Mini kit (QIAGEN, 74104) following manufacturers protocol. Amount and purity (A260/A230 and A260/280 >2.0) of isolated RNA were measured by spectrophotometry (Nanodrop, Thermo Fisher Scientific, Norway), while RNA integrity (RNA integrity number >8) was assessed using Agilent RNA 6000 Nano Kit on a 2100 Bioanalyzer instrument (Agilent Technologies, USA). All 24 samples passed RNA quality control. cDNA libraries were prepared with the TruSeq Stranded mRNA kit (Illumina, USA) following Illumina's protocol. Libraries were quantitated by qPCR and validated using Agilent High Sensitivity DNA Kit on a Bioanalyzer. Libraries were normalized to 22 pM and subjected to cluster and paired-end sequencing was performed on a HiSeq2500 instrument (Illumina, USA), according to the manufacturer's instructions. RNA-seq alignment was performed with the STAR software. Differences in gene expression were determined by a Benjamini and Hochberg's False Discovery Rate (FDR) of 5% or less. RNA-seq was performed at the Genomics Core Facility (GCF), Norwegian University of Science and Technology (NTNU). GCF is funded by the Faculty of Medicine and Health Sciences at NTNU and Central Norway Regional Health Authority.

ATAC sequencing library preparation and analyses. We adapted the previously published Omni-ATACseq protocol to C2C12 cells. In brief, C2C12 cells were cultured until reaching confluency and after several washes with PBS 50,000 cells were pelleted at 4° C. for 5 min and resuspended in a buffer consisting of 0.1% NP40 (Sigma/Roche cat #11332473001), 0.1% Tween-20 (Sigma/Roche cat #11332465001), and 0.01% Digitonin (Promega cat #G9441). Cells were pelleted again at 4° C. for 10 min and resuspended in a transposition mixture containing TD buffer (15027866, Nextera DNA Flex Library Prep), 100 nM transposase (15027865 Nextera DNA Flex Library Prep), PBS, 1% digitonin (Promega cat #G9441), 10% Tween-20 (Sigma/Roche cat #11332465001), and H2O. After incubation for 30 min at 37° C. at 1000 RPM mixing, samples were cleaned up using Zymo DNA Clean and Concentrator-5 Kit (cat #D4014) prior to 5 rounds of PCR amplification using the NEBNext 2× MasterMix (NEB #M0541 L) together with i5 and i7 primers. Our library was quantified with the KAPA Library Quantification kit (cat #KK4854) according to the manufacturer's protocol. Sequencing was performed on a HiSeq2000 system (Illumina) with >63 million pair-end reads with 75 bp read length per sample. Quality and adaptor trimming were performed using TrimGalore and Cutadapt. Phred score cut off of 20 was used and all reads over 20 bp long containing less than 10.0 percent errors were retained. If one read of a pair failed to meet the requirements both were removed, retaining only matching pairs. Filtered reads were aligned to the mouse mm10 genome using bowtie2. Duplicates were marked and removed using Picard Tools Mark Duplicates. Mitochondrial regions, non-unique alignments, non-primary alignments were removed all while retaining properly paired reads (Samtools). Peak calling was carried out using MAC2 in BAMPE mode for all samples. Blacklisted regions were removed using BEDTools intersect. Using the TxDb.Mmusculus.UCSC.mm10.known Gene library transcription start site (TSS) regions were determined. ATAC-seq signal of TSSs for open-, mono- and di-nucleosome signal profiles were produced using the soGGi library and only nucleosome free reads (<100 bp) were used. Peaks were then annotated using the annotatePeak tool of ChIPseeker R library. Annotation was carried out on all regions and also on TSS regions only. The R package Rsubread was used to count paired reads aligning to the regions defined by our consensus peak set. Reads were filtered to retain only those that aligned to peaks present in at least 3 replicates. The DESeq2 R package was used for the identification of differentially accessible genomic regions. Promoter regions (TSS+/−500 bp) were extracted from the TxDb.Mmusculus.UCSC.mm10.knownGene database library. Genes names were annotated using the annotatePeak function from the ChIPseeker package. The function enrichGO from the R package clusterProfiler was used to conduct overrepresentation analysis of GO terms using the whole genome as background. Motif searches were carried out using the findMotifsGenome.pl script from the HOMER suite of tools.

RNA-Fluorescence in situ hybridization. Human skeletal muscle cells (Lonza, Switzerland) were cultured on a 18 mm round glass coverslip (Marienfeld GmbH) in a 12 well plate and fixated with 4% formaldehyde for 10 min. After permeabilization of cells with 70% ethanol cells were washed with Wash Buffer A from (SMF-WA1-60; StellarisR) incubated in with the probes diluted in Hybridization buffer (SMF-HB1-10; StellarisR) at 37° C. overnight in the dark. GAPDH probes (SMF-2019-1; StellarisR) was used as a positive control for cytosolic RNA, MALAT1 for nuclear RNA (SMF-2046-1; StellarisR). After washing with Wash Buffer A coverslips were incubated with DAPI for 20 min in the dark before washing with Wash Buffer B (SMF-WB1-20; StellarisR) and mounting (Fluoromount G, SouthernBiotech) coverslips and imaging with a fluorescence microscope (DM5500, Leica).

Luciferase assay. The 25 base pairs surrounding the variants rs74924495, rs72624662 and rs74360724 of the minor (A) or major (G) allele (table S6) were synthesized (Genewiz) and subcloned into the pGL3-promoter vector (Promega) using the Xbal restriction enzyme (NEB, R0145) and sequenced to check the correct directionality of the inserted sequence. For the luciferase assay, human muscle cells (Lonza, Switzerland) were seeded in 96 well plates and 24 h after transfected with 100 μg of either the pGL3-promoter (Promega, E1761) or pGL3-rs74924495-G, pGL3-rs74924495-A, pGL3-rs72624662-G, pGL3-rs72624662-A, pGL3-rs74360724-G, or pGL3-rs74360724-A plasmid. In all conditions, cells were co-transfected with 10 μg of pRL-TK Renilla expressing vector. TransiT (Mirus) was used as transfection reagent, following manufacturer's instructions. Cells were analyzed for both Luciferase and Renilla luminescence using the Dual-GloR Luciferase Assay System Protocol (Promega, E2920), according to the manufacturer's instructions. Experiments were repeated at least twice.

Human datasets. Gene expression analysis of young vs. old human muscle biopsies was obtained from publicly available microarray dataset GSE25941. Young (n=8) and old (n=11) healthy female participants who have never been involved in any formal exercise were analyzed. Skeletal muscle biopsies were obtained from the vastus lateralis in the basal state and analyzed using the Affymetrix Human Genome U133 Plus 2.0 Array platform. Differential gene expression in this microarray dataset was analyzed with GEO2R, a bioinformatics tool from NCBI (https://www.ncbi.nlm.nih.gov/geo/info/geo2r.html) that allows the comparison of groups of samples from the GEO database. We verified that information provided by the data submitter was sufficient to identify the group allocation, and performed differential gene expression analysis.

Bioinformatic analyses. The public available RNA sequencing dataset GSE71972 was analyzed by aligning the raw sequencing data to the human genome using STAR and to the human transcriptome using kallisto. Protein-coding genes and pseudogenes were excluded from both analyses. To get robust differentially expressed long noncoding RNAs, results from the two aligning methods were overlapped (Table 1). Cytor expression in the type I muscle soleus (n=10) compared to the type II muscle EDL (n=11) was analyzed in the public available dataset GSE112716. Cytor was identified by the probe ID 47174 which corresponds to Gm14005. Outliers with expression values <10 were removed from the analysis. The chromatin immunoprecipitation tracks of H3K27ac and H3K4me1 collected in human primary myoblasts were extracted from GSE126099 and the inventor's published threshold was used to define enhancer elements where there was an overlap between H3K27ac and H3K4me1.

1. A method of:
a) treating a subject having or being at risk of developing skeletal muscle atrophy, starvation, sarcopenia, cachexia (e.g. cancer cachexia or AIDS cachexia), sepsis, diabetes (particularly type I diabetes), muscular dystrophy (e.g. Duchenne Muscular Dystrophy, Becker's MD, Congenital MD, Myotonic dystrophy (e.g. type I and type II), Limb-girdle MD, Emery-Dreifuss MD, Distal MD and Facioscapulohumeral Muscular Dystrophy), Chronic Heart Failure (e.g. diaphragm muscle atrophy resulting from Chronic Heart Failure), Chronic Obstructive Pulmonary Disease (e.g. diaphragm muscle atrophy resulting from Chronic Obstructive Pulmonary Disease), Amyotrophic Lateral Sclerosis (ALS), Spinal Muscular Atrophy, Guillain-Barré syndrome, intensive care unit acquired weakness, immobilization resulting from bone fractures (e.g. hip (femur neck) fractures) or immobilization resulting from an infection;
b) promoting myogenesis in skeletal muscle of a subject, wherein the subject has or is at risk of developing skeletal muscle atrophy, starvation, sarcopenia, cachexia (e.g. cancer cachexia or AIDS cachexia), sepsis, diabetes (particularly type I diabetes), muscular dystrophy (e.g. Duchenne Muscular Dystrophy, Becker's MD, Congenital MD, Myotonic dystrophy (e.g. type I and type II), Limb-girdle MD, Emery-Dreifuss MD, Distal MD and Facioscapulohumeral Muscular Dystrophy), Chronic Heart Failure (e.g. diaphragm muscle atrophy resulting from Chronic Heart Failure), Chronic Obstructive Pulmonary Disease (e.g. diaphragm muscle atrophy resulting from Chronic Obstructive Pulmonary Disease), Amyotrophic Lateral Sclerosis (ALS), Spinal Muscular Atrophy, Guillain-Barré syndrome, intensive care unit acquired weakness, immobilization resulting from bone fractures (e.g. hip (femur neck) fractures) or immobilization resulting from an infection;
c) increasing skeletal muscle mass in a subject, wherein the subject has or is at risk of developing skeletal muscle atrophy, starvation, sarcopenia, cachexia (e.g. cancer cachexia or AIDS cachexia), sepsis, diabetes (particularly type I diabetes), muscular dystrophy (e.g. Duchenne Muscular Dystrophy, Becker's MD, Congenital MD, Myotonic dystrophy (e.g. type I and type II), Limb-girdle MD, Emery-Dreifuss MD, Distal MD and Facioscapulohumeral Muscular Dystrophy), Chronic Heart Failure (e.g. diaphragm muscle atrophy resulting from Chronic Heart Failure), Chronic Obstructive Pulmonary Disease (e.g. diaphragm muscle atrophy resulting from Chronic Obstructive Pulmonary Disease), Amyotrophic Lateral Sclerosis (ALS), Spinal Muscular Atrophy, Guillain-Barré syndrome, intensive care unit acquired weakness, immobilization resulting from bone fractures (e.g. hip (femur neck) fractures) or immobilization resulting from an infection;
d) improving skeletal muscle function in a subject, wherein the subject has or is at risk of developing starvation, sarcopenia, cachexia (e.g. cancer cachexia or AIDS cachexia), sepsis, diabetes (particularly type I diabetes), muscular dystrophy (e.g. Duchenne Muscular Dystrophy, Becker's MD, Congenital MD, Myotonic dystrophy (e.g. type I and type II), Limb-girdle MD, Emery-Dreifuss MD, Distal MD and Facioscapulohumeral Muscular Dystrophy), Chronic Heart Failure (e.g. diaphragm muscle atrophy resulting from Chronic Heart Failure), Chronic Obstructive Pulmonary Disease (e.g. diaphragm muscle atrophy resulting from Chronic Obstructive Pulmonary Disease), Amyotrophic Lateral Sclerosis (ALS), Spinal Muscular Atrophy, Guillain-Barré syndrome, intensive care unit acquired weakness, immobilization resulting from bone fractures (e.g. hip (femur neck) fractures) or immobilization resulting from an infection; or
e) preventing or treating skeletal muscle atrophy and/or skeletal muscle dysfunction in a subject, optionally wherein the subject has or is at risk of developing starvation, sarcopenia, cachexia (e.g. cancer cachexia or AIDS cachexia), sepsis, diabetes (particularly type I diabetes), muscular dystrophy (e.g. Duchenne Muscular Dystrophy, Becker's MD, Congenital MD, Myotonic dystrophy (e.g. type I and type II), Limb-girdle MD, Emery-Dreifuss MD, Distal MD and Facioscapulohumeral Muscular Dystrophy), Chronic Heart Failure (e.g. diaphragm muscle atrophy resulting from Chronic Heart Failure), Chronic Obstructive Pulmonary Disease (e.g. diaphragm muscle atrophy resulting from Chronic Obstructive Pulmonary Disease), Amyotrophic Lateral Sclerosis (ALS), Spinal Muscular Atrophy, Guillain-Barré syndrome, intensive care unit acquired weakness, immobilization resulting from bone fractures (e.g. hip (femur neck) fractures) or immobilization resulting from an infection; or
by administering to the subject a Cytor agonist and/or a Tead1 antagonist,
wherein the Cytor agonist
(i) comprises RNA comprising a nucleotide sequence as set forth in any one of SEQ ID NOs: 1-15 or a nucleotide sequence with at least 80% sequence identity to a sequence as set forth in any one of SEQ ID NOs: 1-15, wherein the RNA promotes myogenesis in skeletal muscle;
(ii) comprises a nucleic acid molecule encoding Cytor RNA or an orthologue thereof or a functionally equivalent fragment or variant of said Cytor RNA or orthologue, wherein the nucleic acid molecule comprises a nucleotide sequence as set forth in any one of SEQ ID NOs: 20-34 or a nucleotide sequence with at least 80% sequence identity to a sequence as set forth in any one of SEQ ID NOs: 20-34, wherein the nucleic acid molecule encodes an RNA that promotes myogenesis in skeletal muscle; or
(iii) comprises a nucleic acid molecule encoding a protein that induces expression of endogenous Cytor RNA, wherein the protein comprises a domain that binds to the Cytor promoter operably linked to a transcriptional activator, and wherein the protein comprising a domain that binds to the Cytor promoter is a CRISPR associated protein (e.g. a nuclease-deficient CRISPR associated protein, e.g. dCas9) and the agonist further comprises a guide RNA capable of hydridising with the Cytor promoter nucleic acid; and/or
wherein the Tead1 antagonist
(i′) is verteporfin;
(ii′) is an inhibitor, wherein the inhibitor is an aptamer, a ribozyme, a siRNA, a shRNA, an antisense oligonucleotide, an antibody or an antibody mimetic, wherein the antibody mimetic is preferably selected from affibodies, adnectins, anticalins, DARPins, avimers, nanofitins, affilins, Kunitz domain peptides, Fynomers, trispecific binding molecules and probodies; or
(iii′) is a nucleotide based inhibitor comprising
(a) a nucleic acid sequence which comprises or consists of a nucleic acid sequence being complementary to at least 12 continuous nucleotides of a nucleic acid sequence selected from SEQ ID NOs: 39 to 42,
(b) a nucleic acid sequence which comprises or consists of a nucleic acid sequence which is at least 70% identical to the complementary strand of one or more nucleic acid sequences selected from SEQ ID NOs: 39 to 42,
(c) a nucleic acid sequence which comprises or consists of a nucleic acid sequence according to (a) or (b), wherein the nucleic acid sequence is DNA or RNA, or
(d) an expression vector expressing the nucleic acid sequence as defined in any one of (a) to (c), preferably under the control of a skeletal muscle-specific promoter.

2-5. (canceled)

6. The method of claim 1, wherein the subject has or is at risk of skeletal muscle atrophy.

7. The method of claim 1, wherein the subject is heterozygous or homozygous for the G allele of cis-eQTL rs74360724.

8. The method of claim 1, wherein the subject (i) is at least 45 years old, (ii) is inactive or immobile, and/or (iii) has or is at risk of developing sarcopenia.

9. The method of claim 1, wherein the agonist comprises a nanoparticle containing the RNA or the nucleic acid molecule as defined in claim 1, item (ii).

10. (canceled)

11. (canceled)

12. The method of claim 1, wherein the agonist increases the amount of Cytor RNA in skeletal muscle directly.

13. (canceled)

14. The method of claim 1, wherein the agonist comprises RNA comprising a nucleotide sequence as set forth in any one of SEQ ID NOs:1-15 or a nucleotide sequence with at least 80% sequence identity to a sequence as set forth in any one of SEQ ID NOs:1-15.

15. The method of claim 9, wherein the nanoparticle is a liposome.

16-19. (canceled)

20. The method of claim 1, wherein the nucleic acid molecule comprises a nucleotide sequence as set forth in any one of SEQ ID NOs:20-34 or a nucleotide sequence with at least 80% sequence identity to a sequence as set forth in any one of SEQ ID NOs:20-34.

21. The method of claim 9, wherein the nanoparticle is a viral vector.

22-24. (canceled)

25. The method of claim 1, wherein the agonist inhibits degradation of endogenous Cytor RNA.

26. The method of claim 1, wherein the agonist that inhibits degradation of endogenous Cytor RNA is an RNA binding protein.

27. The method of claim 1, wherein the agonist is a small molecule.

28. The method of claim 1, wherein the antagonist inhibits the expression and/or the activity of Tead1.

29-31. (canceled)

32. A modified farmed animal or farmed fish which has an increased amount of Cytor RNA or a decreased amount of or no Tead1 RNA in its skeletal muscle in comparison to a corresponding unmodified non-human animal, preferably wherein the animal has been genetically-modified to increase the expression of Cytor RNA and/or to decrease or prevent the expression of Tead1 RNA.

33. The modified farmed animal or farmed fish of claim 32, wherein the farmed animal is a poultry, pig, cattle, sheep or goat or the farmed fish is a salmon, tilapia or tuna.

34-36. (canceled)

37. A method for producing the modified farmed animal or farmed fish of claim 32, the method comprising:
(i) providing a cell of a farmed animal or farmed fish which has been genetically-modified such that myoblast cells derived from said cell express an increased amount of Cytor RNA and/or a decreased amount of or no Tead1 RNA or protein compared to unmodified myoblast cells; and
(ii) generating a genetically-modified farmed animal or farmed fish from said genetically-modified cell of a farmed animal or farmed fish.

38. An in vitro method for:
a) producing muscle fibres comprising culturing modified myoblasts which have increased amounts of Cytor RNA and/or a decreased amount of or no Tead1 RNA or protein in comparison to unmodified myoblasts under conditions suitable to produce muscle fibres;
b) determining the risk of developing skeletal muscle atrophy (e.g. sarcopenia) in a subject comprising determining the genotype of the subject
at the cis-eQTL rs74360724, wherein when the subject is heterozygous or homozygous for the G allele they have an increased risk of developing muscle atrophy (e.g. sarcopenia) compared to a subject that is homozygous for the A allele, and/or
at the cis-eQTL rs79200838, wherein when the subject is heterozygous or homozygous for the T allele they have an decreased risk of developing muscle atrophy (e.g. sarcopenia) compared to a subject that is homozygous for the C allele;
c) predicting the performance of a subject in an activity associated with fast-twitch muscle (e.g. an athlete, e.g. a sprinter) comprising determining the genotype of the subject
at the cis-eQTL rs74360724, wherein when the subject is heterozygous or homozygous for the A allele they have an increased likelihood of outperforming a subject that is homozygous for the G allele, and/or
at the cis-eQTL rs79200838, wherein when the subject is heterozygous or homozygous for the C allele they have an decreased of outperforming a subject that is homozygous for the T allele;
d) predicting the capability of a subject to produce fast-twitch muscle comprising determining the genotype of the subject
at the cis-eQTL rs74360724, wherein when the subject is heterozygous or homozygous for the A allele they have an increased capability of producing fast-twitch muscle compared a subject that is homozygous for the G allele, and/or
at the cis-eQTL rs79200838, wherein when the subject is heterozygous or homozygous for the C allele they have an decreased capability of producing fast-twitch muscle compared a subject that is homozygous for the T allele.

39. The in vitro method of claim 38, wherein the myoblasts have been genetically-modified to increase the expression of Cytor RNA and/or to decrease the expression of Tead1 RNA or protein.

40. The in vitro method of claim 38, wherein the myoblasts comprise a nucleic acid molecule encoding Cytor RNA operably linked to a heterologous promoter and/or a mutated or deleted gene encoding Tead1 RNA.

41-46. (canceled)
